(19) United States
(12) Patent Application Publication
(10) Pub. No.: US 2023/0190903 A1
(43) Pub. Date: Jun. 22, 2023

(54) PROSTATE CANCER VACCINES AND USES THEREOF

(71) Applicant: Janssen Biotech, Inc., Horsham, PA (US)

(72) Inventors: Patrick WILKINSON, Collegeville, PA (US); Manuel Alejandro SEPULVEDA, West Windsor, NJ (US); Charles George DRAKE, New Hope, PA (US); Derick Siegel, Telford, PA (US)

(21) Appl. No.: 18/080,634

(22) Filed: Dec. 13, 2022

Related U.S. Application Data
(60) Provisional application No. 63/290,164, filed on Dec. 16, 2021.

(51) Int. Cl. A61K 39/00; A61K 39/395; A61P 35/00

(52) U.S. Cl. CPC A61K 39/001193; A61K 39/3955; A61P 35/00; A61K 2039/5256; A61K 2039/70; A61K 2039/545

(57) ABSTRACT

Disclosed herein are methods of treating or preventing prostate cancer in a subject, the methods comprising administering to the subject a treatment regimen comprising two or more vaccines comprising a great ape adenovirus serotype 20 (GAd20) virus that, in turn, comprises a nucleotide sequence encoding the amino acid sequence of SEQ ID NO: 1 and one or more vaccines comprising a Modified Vaccinia Ankara (MVA) virus that, in turn, comprises a nucleotide sequence encoding the amino acid sequence of SEQ ID NO: 3 to thereby treat or prevent the prostate cancer.

### CROSS-REFERENCE TO RELATED APPLICATIONS

This application claims priority to U.S. Provisional Application Ser. No. 63/290,164, filed on Dec. 16, 2021, the disclosure of which is hereby incorporated by reference in its entirety.

### SEQUENCE LISTING

The contents of the electronic sequence listing (JBI6595USNP1_Sequence Listing.xml; Size: 111,776 bytes; and Date of Creation: Nov. 23, 2022) is herein incorporated by reference in its entirety.

### BACKGROUND

Prostate cancer is the most common non-cutaneous malignancy in men and the second leading cause of death in men from cancer in the western world. Prostate cancer results from the uncontrolled growth of abnormal cells in the prostate gland. Once a prostate cancer tumor develops, androgens such as testosterone promote prostate cancer growth. At its early stages, localized prostate cancer is often curable with local therapy including, for example, surgical removal of the prostate gland and radiotherapy. However, when local therapy fails to cure prostate cancer, as it does in up to a third of men, the disease progresses into incurable metastatic disease.

For many years, the established standard of care for men with malignant castration-resistant prostate cancer (mCRPC) was docetaxel chemotherapy. More recently, abiraterone acetate (ZYTIGA®) in combination with prednisone has been approved for treating metastatic castrate resistant prostate cancer. Androgen receptor (AR)-targeted agents, such as enzalutamide (XTANDI®) have also entered the market for treating metastatic castrate resistant prostate cancer. Platinum-based chemotherapy has been tested in a number of clinical studies in molecularly unselected prostate cancer patients with limited results and significant toxicities. However, there remains a subset of patients who either do not respond initially or become refractory (or resistant) to these treatments. No approved therapeutic options are available for such patients.

### SUMMARY

Disclosed herein are methods of treating or preventing prostate cancer in a subject, the methods comprising administering to the subject a treatment regimen comprising two or more vaccines comprising a great ape adenovirus serotype 20 (GAd20) virus that, in turn, comprises a nucleotide sequence encoding the amino acid sequence of SEQ ID NO: 1 and one or more vaccines comprising a Modified Vaccinia Ankara (MVA) virus that, in turn, comprises a nucleotide sequence encoding the amino acid sequence of SEQ ID NO: 3 to thereby treat or prevent the prostate cancer.

Also provided are methods of treating or preventing prostate cancer in a subject, the methods comprising administering to the subject: a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1; a vaccine comprising 1×10⁸ infectious units (IFU) of an MVA virus at about week 9, wherein the MVA virus comprises a nucleotide sequence encoding the amino acid sequence of SEQ ID NO: 3; a vaccine comprising 1×10¹¹ VP of the GAd20 virus at about week 15 and about week 18; a vaccine comprising 1×10⁸ IFU of the MVA virus at about week 24; and a vaccine comprising 1×10⁸ IFU of the MVA virus at about week 36, about week 48, and about week 60.

Methods of treating or preventing prostate cancer in a subject are disclosed, wherein the methods comprise administering to the subject: a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1; a vaccine comprising 1×10⁸ infectious units (IFU) of an MVA virus at about week 9, wherein the MVA virus comprises a nucleotide sequence encoding the amino acid sequence of SEQ ID NO: 3; a vaccine comprising 1×10¹¹ VP of the GAd20 virus at about week 15; a vaccine comprising 1×10⁸ IFU of the MVA virus at about week 24; and a vaccine comprising 1×10⁸ IFU of the MVA virus at about week 36, about week 48, and about week 60.

Also provided are methods of treating or preventing prostate cancer in a subject, the methods comprising administering to the subject: a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1; a vaccine comprising 1×10⁸ infectious units (IFU) of an MVA virus at about week 9, wherein the MVA virus comprises a nucleotide sequence encoding the amino acid sequence of SEQ ID NO: 3; a vaccine comprising 1×10⁸ IFU of the MVA virus at about week 15, about week 18, and about week 24; and a vaccine comprising 1×10⁸ IFU of the MVA virus at about week 36, about week 48, and about week 60.

Disclosed herein are methods of treating or preventing prostate cancer in a subject, the methods comprising administering to the subject: a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1; a vaccine comprising 1×10⁸ infectious units (IFU) of an MVA virus at about week 9, wherein the MVA virus comprises a nucleotide sequence encoding the amino acid sequence of SEQ ID NO: 3; a vaccine comprising 1×10⁸ IFU of the MVA virus at about week 15 and about week 24; and a vaccine comprising 1×10⁸ IFU of the MVA virus at about week 36, about week 48, and about week 60.

Methods of treating or preventing prostate cancer in a subject are disclosed, wherein the methods comprise administering to the subject: 1 mg/kg to 3 mg/kg of an anti-CTLA4 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1; 1 mg/kg to 3 mg/kg of an anti-CTLA4 antibody and a vaccine comprising 1×10⁸ infectious units (IFU) of an MVA virus at about week 9, wherein the MVA virus comprises a nucleotide sequence encoding the amino acid sequence of SEQ ID NO: 3; 1 mg/kg to 3 mg/kg of an anti-CTLA4 antibody and a vaccine comprising 1×10¹¹ VP of the GAd20 virus at about week 15 and about week 18; 1 mg/kg to 3 mg/kg of an anti-CTLA4 antibody and a vaccine comprising 1×10⁸ IFU of the MVA virus at about week 24; and 1 mg/kg to 3 mg/kg of an anti-CTLA4 antibody and a vaccine comprising 1×10⁸ IFU of the MVA virus at about week 36, about week 48, and about week 60.

Methods of treating or preventing prostate cancer in a subject are provided, wherein the methods comprise administering to the subject: 1 mg/kg to 3 mg/kg of an anti-PD1 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1; 1 mg/kg to 3 mg/kg of an anti-PD1 antibody and a vaccine comprising 1×10⁸ infectious units (IFU) of an MVA virus at about week 9, wherein the MVA virus comprises a nucleotide sequence encoding the amino acid sequence of SEQ ID NO: 3; 1 mg/kg to 3 mg/kg of an anti-PD1 antibody and a vaccine comprising 1×10¹¹ VP of the GAd20 virus at about week 15 and about week 18; 1 mg/kg to 3 mg/kg of an anti-PD1 antibody and a vaccine comprising 1×10⁸ IFU of the MVA virus at about week 24; and 1 mg/kg to 3 mg/kg of an anti-PD1 antibody and a vaccine comprising 1×10⁸ IFU of the MVA virus at about week 36, about week 48, and about week 60.

Also provided are methods of treating or preventing prostate cancer in a subject, the methods comprising administering to the subject: 1 mg/kg to 3 mg/kg of an anti-CTLA4 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1; 1 mg/kg to 3 mg/kg of an anti-CTLA4 antibody and a vaccine comprising 1×10⁸ infectious units (IFU) of an MVA virus at about week 9, wherein the MVA virus comprises a nucleotide sequence encoding the amino acid sequence of SEQ ID NO: 3; 1 mg/kg to 3 mg/kg of an anti-CTLA4 antibody and a vaccine comprising 1×10¹¹ VP of the GAd20 virus at about week 15; 1 mg/kg to 3 mg/kg of an anti-CTLA4 antibody and a vaccine comprising 1×10⁸ IFU of the MVA virus at about week 24; and 1 mg/kg to 3 mg/kg of an anti-CTLA4 antibody and a vaccine comprising 1×10⁸ IFU of the MVA virus at about week 36, about week 48, and about week 60.

Disclosed herein are methods of treating or preventing prostate cancer in a subject, the methods comprising administering to the subject: 1 mg/kg to 3 mg/kg of an anti-PD1 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1; 1 mg/kg to 3 mg/kg of an anti-PD1 antibody and a vaccine comprising 1×10⁸ infectious units (IFU) of an MVA virus at about week 9, wherein the MVA virus comprises a nucleotide sequence encoding the amino acid sequence of SEQ ID NO: 3; 1 mg/kg to 3 mg/kg of an anti-PD1 antibody and a vaccine comprising 1×10¹¹ VP of the GAd20 virus at about week 15; 1 mg/kg to 3 mg/kg of an anti-PD1 antibody and a vaccine comprising 1 x 10⁸ IFU of the MVA virus at about week 24; and 1 mg/kg to 3 mg/kg of an anti-PD1 antibody and a vaccine comprising 1×10⁸ IFU of the MVA virus at about week 36, about week 48, and about week 60.

Disclosed herein are methods of treating or preventing prostate cancer in a subject, the methods comprising administering to the subject: 1 mg/kg to 3 mg/kg of an anti-CTLA4 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1; 1 mg/kg to 3 mg/kg of an anti-CTLA4 antibody and a vaccine comprising 1×10⁸ infectious units (IFU) of an MVA virus at about week 9, wherein the MVA virus comprises a nucleotide sequence encoding the amino acid sequence of SEQ ID NO: 3; 1 mg/kg to 3 mg/kg of an anti-CTLA4 antibody and a vaccine comprising 1×10⁸ IFU of the MVA virus at about week 15, about week 18, and about week 24; and 1 mg/kg to 3 mg/kg of an anti-CTLA4 antibody and a vaccine comprising 1×10⁸ IFU of the MVA virus at about week 36, about week 48, and about week 60.

Provided herein are methods of treating or preventing prostate cancer in a subject, the method comprising administering to the subject: 1 mg/kg to 3 mg/kg of an anti-PD1 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1; 1 mg/kg to 3 mg/kg of an anti-PD1 antibody and a vaccine comprising 1×10⁸ infectious units (IFU) of an MVA virus at about week 9, wherein the MVA virus comprises a nucleotide sequence encoding the amino acid sequence of SEQ ID NO: 3; 1 mg/kg to 3 mg/kg of an anti-PD1 antibody and a vaccine comprising 1×10⁸ IFU of the MVA virus at about week 15, about week 18, and about week 24; and 1 mg/kg to 3 mg/kg of an anti-PD1 antibody and a vaccine comprising 1×10⁸ IFU of the MVA virus at about week 36, about week 48, and about week 60.

Methods of treating or preventing prostate cancer in a subject are disclosed, wherein the methods comprise administering to the subject: 1 mg/kg to 3 mg/kg of an anti-CTLA4 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1; 1 mg/kg to 3 mg/kg of an anti-CTLA4 antibody and a vaccine comprising 1×10⁸ infectious units (IFU) of an MVA virus at about week 9, wherein the MVA virus comprises a nucleotide sequence encoding the amino acid sequence of SEQ ID NO: 3; 1 mg/kg to 3 mg/kg of an anti-CTLA4 antibody and a vaccine comprising 1×10⁸ IFU of the MVA virus at about week 15 and about week 24; and 1 mg/kg to 3 mg/kg of an anti-CTLA4 antibody and a vaccine comprising 1×10⁸ IFU of the MVA virus at about week 36, about week 48, and about week 60.

Provided herein are methods of treating or preventing prostate cancer in a subject, the method comprising administering to the subject: 1 mg/kg to 3 mg/kg of an anti-PD1 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1; 1 mg/kg to 3 mg/kg of an anti-PD1 antibody and a vaccine comprising 1×10⁸ infectious units (IFU) of an MVA virus at about week 9, wherein the MVA virus comprises a nucleotide sequence encoding the amino acid sequence of SEQ ID NO: 3; 1 mg/kg to 3 mg/kg of an anti-PD1 antibody and a vaccine comprising 1×10⁸ IFU of the MVA virus at about week 15 and about week 24; and 1 mg/kg to 3 mg/kg of an anti-PD1 antibody and a vaccine comprising 1×10⁸ IFU of the MVA virus at about week 36, about week 48, and about week 60.

### BRIEF DESCRIPTION OF THE DRAWINGS

The summary, as well as the following detailed description, is further understood when read in conjunction with the appended drawings. For the purpose of illustrating the disclosed methods, there are shown in the drawings exemplary embodiments of the methods; however, the methods is not limited to the specific embodiments disclosed. In the drawings:

FIG. 1A and FIG. 1B illustrate exemplary dosing schedules.

FIG. 2 illustrates the number of cynomolgus monkeys exhibiting prostate cancer neo-antigen specific IFNγ+ T cells (SFU/10⁶ cells) (responders) at week 2 and week 6 after receiving a single dose (“single prime”) or two doses (“double prime”) of the disclosed GAd20 vaccine.

FIG. 3 illustrates the antigen specific T cell response in cynomolgus monkeys after receiving a double prime of the GAd20 vaccine followed by an MVA vaccine boost at the indicated time points.

FIG. 4 illustrates the number of cynomolgus monkeys exhibiting prostate cancer neo-antigen specific IFNy+T cells (SFU/10⁶ cells) (responders) at week 4 after receiving a single dose (“single prime”) of GAd20 alone or in combination with an anti-CTLA4 antibody (αCTLA4).

FIG. 5 illustrates the prostate cancer neo-antigen specific IFNγ+ T cell (SFU/10⁶ cells) response in cynomolgus monkeys up to week 22 after receiving the indicated dosing schedules.

FIG. 6 illustrates the polyfunctional (IFNγ/TNFα double positive) CD8+1 T cell response in cynomolgus monkeys following the administration of GAd20/GAd20/MVA (at weeks 0, 4, 12) alone (circle) or in combination with an anti-CTLA4 antibody (square).

FIG. 7 illustrates the effector memory neo-antigen specific CD8+ T cell response in cynomolgus monkeys following the administration of GAd20/GAd20/MVA (at weeks 0, 4, 12) alone (circle) or in combination with an anti-CTLA4 antibody (square) at week 21.

FIG. 8 illustrates the breadth of the CD8+ T cell response in cynomolgus monkeys following the administration of GAd20/GAd20/MVA (at weeks 0, 4, 12) alone or in combination with an anti-CTLA4 antibody.

### DETAILED DESCRIPTION OF ILLUSTRATIVE EMBODIMENTS

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the methods as described herein. Where a range of numerical values is stated herein as being less than a stated value, the range is nevertheless bounded on its lower end by a non-zero value. It is not intended that the scope of the methods be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “about” is used to encompass variations of ±10% or less, variations of ±5% or less, variations of ±1% or less, variations of ±0.5% or less, or variations of ±0.1% or less from the specified value

The term “comprising” is intended to include examples encompassed by the terms “consisting essentially of” and “consisting of”; similarly, the term “consisting essentially of” is intended to include examples encompassed by the term “consisting of.”

“Administered with” means that two or more therapeutics (such as a virus and an antibody) can be administered to a subject together in a mixture, concurrently as single agents, or sequentially as single agents in any order.

“Treat,” “treatment,” and like terms refer to both therapeutic treatment and prophylactic or preventative measures, and includes reducing the severity and/or frequency of symptoms of prostate cancer, eliminating symptoms and/or the underlying cause of the symptoms of prostate cancer, reducing the frequency or likelihood of symptoms of prostate cancer and/or their underlying cause, and improving or remediating damage caused, directly or indirectly, by prostate cancer. Treatment also includes prolonging survival as compared to the expected survival of a subject not receiving treatment. Subjects to be treated include those that have prostate cancer as well as those prone to have prostate cancer or those in which prostate cancer is to be prevented.

Disclosed herein are methods of treating or preventing prostate cancer in a subject, wherein the prostate cancer comprises one or more prostate cancer neoantigens encoding an amino acid sequence of SEQ ID NO: 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, or 93. The disclosed vaccines (e.g., those comprising a nucleic acid sequence encoding the amino acid sequence of SEQ ID NO: 1 or 3) can be used to treat or prevent prostate cancers that express one or more of these neoantigens. The methods of treating or preventing prostate cancer can comprise administering to the subject a treatment regimen comprising:

two or more vaccines comprising a great ape adenovirus serotype 20 (GAd20) virus that, in turn, comprises a nucleotide sequence encoding the amino acid sequence of SEQ ID NO: 1 and

one or more vaccines comprising a Modified Vaccinia Ankara (MVA) virus that, in turn, comprises a nucleotide sequence encoding the amino acid sequence of SEQ ID NO: 3 to thereby treat or prevent the prostate cancer.

GAd20 is an adenovirus that infects gorilla (Gorilla), and can be isolated from stool samples of the gorilla. The GAd20 can be engineered to comprise at least one functional deletion or a complete removal of a gene product that is essential for viral replication, such as one or more of the adenoviral regions E1, E2 and E4, therefore rendering the adenovirus to be incapable of replication. The deletion of the E1 region may comprise deletion of EIA, EIB 55K or EIB 21K, or any combination thereof. Replication deficient adenoviruses are propagated by providing the proteins encoded by the deleted region(s) in trans by the producer cell by utilizing helper plasmids or engineering the produce cell to express the required proteins. Adenovirus vectors may also have a deletion in the E3 region, which is dispensable for replication, and hence such a deletion does not have to be complemented. The GAd20 of the disclosure may comprise a functional deletion or a complete removal of the E1 region and at least part of the E3 region. The GAd20 may further comprise a functional deletion or a complete removal of the E4 region and/or the E2 region. Suitable producer cells that can be utilized are human retina cells immortalized by E1, e.g. 911 or PER.C6 cells (see, e.g., U.S. Pat. No. 5,994,128), E1-transformed amniocytes (See, e.g., EP 1230354), E1-transformed A549 cells (see e.g. Int. Pat. Publ. No. WO1998/39411, U.S. Pat. No. 5,891,690). The nucleotide sequence encoding the amino acid sequence of SEQ ID NO: 1 may be inserted into a site or region (insertion region) in the viral genome that does not affect virus viability of the resultant recombinant virus. The nucleotide sequence encoding the amino acid sequence of SEQ ID NO: 1 may be inserted into the deleted E1 region in parallel (transcribed 5′ to 3′) or anti-parallel (transcribed in a 3′ to 5′ direction relative to the vector backbone) orientation. In addition, appropriate transcriptional regulatory elements that are capable of directing expression of the nucleotide sequence encoding the amino acid sequence of SEQ ID NO: 1 in the mammalian host cells that the virus is being prepared for use may be operatively linked to the nucleotide sequence. “Operatively linked” sequences include both expression control sequences that are contiguous with the nucleic acid sequences that they regulate and regulatory sequences that act in trans, or at a distance to control the regulated nucleic acid sequence.

Recombinant GAd20 particles may be prepared and propagated according to any conventional technique in the field of the art (e.g., Int. Pat. Publ. No. WO1996/17070) using a complementation cell line or a helper virus, which supplies in trans the missing viral genes necessary for viral replication. The cell lines 293 (Graham et al., 1977, J. Gen. Virol. 36: 59-72), PER.C6 (see e.g. U.S. Pat. No. 5,994,128), E1 A549 and 911 are commonly used to complement E1 deletions. Other cell lines have been engineered to complement defective vectors (Yeh, et al., 1996, J. Virol. 70: 559-565; Kroughak and Graham, 1995, Human Gene Ther. 6: 1575-1586; Wang, et al., 1995, Gene Ther. 2: 775-783; Lusky, et al., 1998, J. Virol. 72: 2022-203; EP 919627 and Int. Pat. Publ. No. WO1997/04119). The GAd20 particles may be recovered from the culture supernatant but also from the cells after lysis and optionally further purified according to standard techniques (e.g., chromatography, ultracentrifugation, as described in Int. Pat. Publ. No. WO1996/27677, Int. Pat. Publ. No. WO1998/00524, Int. Pat. Publ. No. WO1998/26048 and Int. Pat. Publ. No. WO2000/50573). The construction and methods for propagating adenoviral vectors, such as GAd20, are also described in for example, U.S. Pat. Nos. 5,559,099, 5,837,511, 5,846,782, 5,851,806, 5,994,106, 5,994,128, 5,965,541, 5,981,225, 6,040,174, 6,020,191, and 6,113,913.

MVA originates from the dermal vaccinia strain Ankara (Chorioallantois vaccinia Ankara (CVA) virus) that was maintained in the Vaccination Institute, Ankara, Turkey for many years and used as the basis for vaccination of humans. However, due to the often severe post-vaccinal complications associated with vaccinia viruses (VACV), there were several attempts to generate a more attenuated, safer smallpox vaccine.

MVA has been generated by 516 serial passages on chicken embryo fibroblasts of the CVA virus (see Meyer et al., J. Gen. Virol., 72: 1031-1038 (1991) and U.S. Pat. No. 10,035,832). As a consequence of these long-term passages the resulting MVA virus deleted about 31 kilobases of its genomic sequence and, therefore, was described as highly host cell restricted to avian cells (Meyer, H. et al.,; Meisinger-Henschel et al., J. Gen. Virol. 88, 3249-3259, 2007). Comparison of the MVA genome to its parent, CVA, revealed 6 major deletions of genomic DNA (deletion I, II, III, IV, V, and VI), totaling 31,000 basepairs. (Meyer et al., J. Gen. Virol. 72:1031-8 (1991)). It was shown in a variety of animal models that the resulting MVA was significantly avirulent (Mayr, A. & Danner, K. Vaccination against pox diseases under immunosuppressive conditions, Dev. Biol. Stand. 41: 225-34, 1978). Being that many passages were used to attenuate MVA, there are a number of different strains or isolates, depending on the passage number in CEF cells, such as MVA 476 MG/14/78, MVA-571, MVA-572, MVA-574, MVA-575 and MVA-BN. MVA 476 MG/14/78 is described for example in Int. Pat. Publ. No. WO2019/115816A1. MVA-572 strain was deposited at the European Collection of Animal Cell Cultures (“ECACC”), Health Protection Agency, Microbiology Services, Porton Down, Salisbury SP4 OJG, United Kingdom (“UK”), under the deposit number ECACC 94012707 on Jan. 27, 1994. MVA-575 strain was deposited at the ECACC under deposit number ECACC 00120707 on Dec. 7, 2000; MVA-Bavarian Nordic (“MVA-BN”) strain was deposited at the ECACC under deposit number V00080038 on Aug. 30, 2000. The genome sequences of MVA-BN and MVA-572 are available at GenBank (Accession numbers DQ983238 and DQ983237, respectively). The genome sequences of other MVA strains can be obtained using standard sequencing methods.

The MVA can be derived from any MVA strain or further derivatives of the MVA strain. A further exemplary MVA strain is deposit VR-1508, deposited at the American Type Culture collection (ATCC), Manassas, Va. 20108, USA. “Derivatives” of MVA refer to viruses exhibiting essentially the same characteristics as the parent MVA, but exhibiting differences in one or more parts of their genomes. In some embodiments, the MVA vector is derived from MVA 476 MG/14/78 . In some embodiments, the MVA vector is derived from MVA-571. In some embodiments, the MVA vector is derived from MVA-572. In some embodiments, the MVA vector is derived from MVA-574. In some embodiments, the MVA vector is derived from MVA-575. In some embodiments, the MVA vector is derived from MVA-BN.

The nucleotide sequence encoding the amino acid sequence of SEQ ID NO: 3 may be inserted into a site or region (insertion region) in the MVA viral genome that does not affect virus viability of the resultant recombinant virus. Such regions can be readily identified by testing segments of virus DNA for regions that allow recombinant formation without seriously affecting virus viability of the recombinant virus. The thymidine kinase (TK) gene is an insertion region that may be used and is present in many viruses, such as in all examined poxvirus genomes. Additionally, MVA contains 6 natural deletion sites, each of which may be used as insertion sites (e.g. deletion I, II, III, IV, V, and VI; see e.g. U.S. Pat. Nos. 5,185,146 and 6.440,442). One or more intergenic regions (IGR) of the MVA may also be used as an insertion site, such as IGRs IGR07/08, IGR 44/45, IGR 64/65, IGR 88/89, IGR 136/137, and IGR 148/149 (see e.g. U.S. Pat. Publ. No. 2018/0064803). Additional suitable insertion sites are described in Int. Pat. Publ. No. WO2005/048957.

MVA virus can be prepared as previously described (Piccini, et al., 1987, Methods of Enzymology 153: 545-563; U.S. Pat. NoS. 4,769,330; 4,772,848; 4,603,112; 5,100,587 and 5,179,993). In an exemplary method, the DNA sequence to be inserted into the viral genome can be placed into an E. coli plasmid construct into which DNA homologous to a section of DNA of the MVA has been inserted. Separately, the DNA sequence to be inserted can be ligated to a promoter. The promoter-gene linkage can be positioned in the plasmid construct so that the promoter-gene linkage is flanked on both ends by DNA homologous to a DNA sequence flanking a region of MVA DNA containing a non-essential locus. The resulting plasmid construct can be amplified by propagation within E. coli bacteria and isolated. The isolated plasmid containing the DNA gene sequence to be inserted can be transfected into a cell culture, e.g., of chicken embryo fibroblasts (CEFs), at the same time the culture is infected with MVA. Recombination between homologous MVA DNA in the plasmid and the viral genome, respectively, can generate an MVA modified by the presence of foreign DNA sequences. MVA particles may be recovered from the culture supernatant or from the cultured cells after a lysis step (e.g., chemical lysis, freezing/thawing, osmotic shock, sonication and the like). Consecutive rounds of plaque purification can be used to remove contaminating wild type virus. Viral particles can then be purified using the techniques known in the art (e.g., chromatographic methods or ultracentrifugation on cesium chloride or sucrose gradients).

The methods can further comprise administering one or more vaccines comprising the GAd20 virus, one or more vaccines comprising the MVA virus, or one or more vaccines comprising the GAd20 virus and one or more vaccines comprising the MVA virus. In some embodiments, the methods can further comprise administering the treatment regimen two or more times. After the initial treatment regimen, for example, the methods can comprise administering two vaccines comprising the GAd20 virus and one vaccine comprising the MVA virus. In some embodiments, the methods can further comprise administering one vaccine comprising the GAd20 virus and one vaccine comprising the MVA virus. In some embodiments, the methods can further comprise administering one or more vaccines comprising the MVA virus. The methods can further comprise, for example, administering three vaccines comprising the MVA virus. The methods can further comprise, for example, administering two vaccines comprising the MVA virus.

Suitable amounts of the GAd20 virus can comprise about 1×10⁹ viral particles (VP) to about 1×10¹³ VP of the GAd20 virus.

Suitable amounts of the MVA virus can comprise about 1×10⁶ infectious units (IFU) to about 1×10¹⁰ IFU of the MVA virus.

The methods can further comprise administering an anti-CTLA4 antibody. The anti-CTLA4 antibody can be administered with the vaccines comprising the GAd20 virus, with the vaccines comprising the MVA virus, or both. Suitable amounts of the anti-CTLA4 antibody comprise about 0.5 mg/kg to about 5 mg/kg. Any antagonistic anti-CTLA4 antibody can be used in the disclosed methods. Suitable anti-CTLA4 antibodies for use in the disclosed methods include, without limitation, human anti-CTLA4 antibodies, mouse anti-CTLA4 antibodies, mammalian anti-CTLA4 antibodies, humanized anti-CTLA4 antibodies, monoclonal anti-CTLA4 antibodies, polyclonal anti-CTLA4 antibodies, and chimeric anti-CTLA4 antibodies. Non-limiting examples of anti-CTLA4 antibodies include Ipilimumab and tremelimumab.

The methods can further comprise administering an anti-PD-1 antibody. The anti-PD-1 antibody can be administered with the vaccines comprising the GAd20 virus, with the vaccines comprising the MVA virus, or both. Suitable amounts of the anti-PD-1 antibody comprise about 0.5 mg/kg to about 5 mg/kg. Any antagonistic anti-PD-1 antibody can be used in the disclosed methods. Suitable anti-PD-1 antibodies for use in the disclosed methods include, without limitation, human anti-PD-1 antibodies, mouse anti-PD-1 antibodies, mammalian anti-PD-1 antibodies, humanized anti-PD-1 antibodies, monoclonal anti-PD-1 antibodies, polyclonal anti-PD-1 antibodies, and chimeric anti-PD-1 antibodies. Non-limiting examples of anti-PD-1 antibodies include cetrelimab, pembrolizumab, nivolumab, sintilimab, cemiplimab, toripalimab, camrelizumab, tislelizumab, dostralimab, spartalizumab, prolgolimab, balstilimab, budigalimab, sasanlimab, avelumab, atezolizumab, durvalumab, envafolimab, and iodapolimab.

The methods can comprise administering a vaccine comprising the GAd20 virus at week 0 and about week 3 and administering a vaccine comprising the MVA virus at about week 9, and:
Administering a vaccine comprising the GAd20 virus at about week 15 and about week 18 and administering a vaccine comprising the MVA virus at about week 24;
Administering a vaccine comprising the GAd20 virus at about week 15 and administering a vaccine comprising the MVA virus at about week 24;
Administering a vaccine comprising the MVA virus at about week 15, about week 18, and about week 24; or
Administering a vaccine comprising the MVA virus at about week 15 and about week 24.

In some embodiments, the methods further comprise administering a vaccine comprising the MVA virus at about week 36, about week 48, and about week 60. The methods can further comprise administering one or more further vaccines comprising the MVA virus.

The methods can comprise administering a vaccine comprising 1×10¹¹ VP of the GAd20 virus at week 0 and about week 3 and administering a vaccine comprising 1×10⁸ IFU of the MVA virus at about week 9, and:
Administering a vaccine comprising 1×10¹¹ VP of the GAd20 virus at about week 15 and about week 18 and administering a vaccine comprising 1×10⁸ IFU of the MVA virus at about week 24;
Administering a vaccine comprising 1×10¹¹ VP of the GAd20 virus at about week 15 and administering a vaccine comprising 1×10⁸ IFU of the MVA virus at about week 24;
Administering a vaccine comprising 1×10⁸ IFU of the MVA virus at about week 15, about week 18, and about week 24; or
Administering a vaccine comprising 1×10⁸ IFU of the MVA virus at about week 15 and about week 24.

In some embodiments, the methods further comprise administering a vaccine comprising 1×10⁸ IFU of the MVA virus at about week 36, about week 48, and about week 60. The methods can further comprise administering one or more further vaccines comprising 1×10⁸ IFU of the MVA virus.

The disclosed methods can comprise the exemplary treatment schedules provided in Table 1 below:

TABLE 1
Exemplary treatment schedules

| | Cycle 1* | | | Cycle 2 | | | Maintenance | | | | |
|---|---|---|---|---|---|---|---|---|---|---|---|
| | Dose 1 | Dose 2 | Dose 3 | Dose 1 | Dose 2 | Dose 3 | Dose 1 | Dose 2 | Dose 3 | | |
| | (wk 0) | (wk 3) | (wk 9) | (wk 15) | (wk 18) | (wk 24) | (wk 36) | (wk 48) | (wk 60) | Dose 4 | Dose 5 |
| 1 | GAd20 | GAd20 | MVA | GAd20 | GAd20 | MVA | MVA | MVA | MVA | MVA | MVA |
| 2 | GAd20 | GAd20 | MVA | GAd20 | | MVA | MVA | MVA | MVA | MVA | MVA |
| 3 | GAd20 | GAd20 | MVA | MVA | MVA | MVA | MVA | MVA | MVA | MVA | MVA |
| 4 | GAd20 | GAd20 | MVA | MVA | | MVA | MVA | MVA | MVA | MVA | MVA |

*Cycle 1 is referred to herein as a “treatment regimen.”

Disclosed herein are methods of treating or preventing prostate cancer in a subject, the method comprising administering to the subject:

a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1,

a vaccine comprising 1×10⁸ infectious units (IFU) of an MVA virus at about week 9, wherein the MVA virus comprises a nucleotide sequence encoding the amino acid sequence of SEQ ID NO: 3;

a vaccine comprising 1×10¹¹ VP of the GAd20 virus at about week 15 and about week 18,

a vaccine comprising 1×10⁸ IFU of the MVA virus at about week 24; and

a vaccine comprising 1×10⁸ IFU of the MVA virus at about week 36, about week 48, and about week 60.

Disclosed herein are methods of treating or preventing prostate cancer in a subject, the method comprising administering to the subject:

a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1,

a vaccine comprising 1×10⁸ infectious units (IFU) of an MVA virus at about week 9, wherein the MVA virus comprises a nucleotide sequence encoding the amino acid sequence of SEQ ID NO: 3;

a vaccine comprising 1×10¹¹ VP of the GAd20 virus at about week 15,

a vaccine comprising 1×10⁸ IFU of the MVA virus at about week 24; and

a vaccine comprising 1×10⁸ IFU of the MVA virus at about week 36, about week 48, and about week 60.

Disclosed herein are methods of treating or preventing prostate cancer in a subject, the method comprising administering to the subject:

a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1,

a vaccine comprising 1×10⁸ infectious units (IFU) of an MVA virus at about week 9, wherein the MVA virus comprises a nucleotide sequence encoding the amino acid sequence of SEQ ID NO: 3;

a vaccine comprising 1×10⁸ IFU of the MVA virus at about week 15, about week 18, and about week 24; and

a vaccine comprising 1×10⁸ IFU of the MVA virus at about week 36, about week 48, and about week 60.

Disclosed herein are methods of treating or preventing prostate cancer in a subject, the method comprising administering to the subject:

a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1,

a vaccine comprising 1×10⁸ infectious units (IFU) of an MVA virus at about week 9, wherein the MVA virus comprises a nucleotide sequence encoding the amino acid sequence of SEQ ID NO: 3;

a vaccine comprising 1×10⁸ IFU of the MVA virus at about week 15 and about week 24; and

a vaccine comprising 1×10⁸ IFU of the MVA virus at about week 36, about week 48, and about week 60.

Any of the above methods can further comprise administering an anti-CTLA4 antibody. The anti-CTLA4 antibody can be administered with the vaccines comprising the GAd20 virus, with the vaccines comprising the MVA virus, or both. Suitable amounts of the anti-CTLA4 antibody comprise about 1 mg/kg to about 3 mg/kg.

Any of the above methods can further comprise administering an anti-PD-1 antibody. The anti-PD-1 antibody can be administered with the vaccines comprising the GAd20 virus, with the vaccines comprising the MVA virus, or both. Suitable amounts of the anti-PD-1 antibody comprise about 1 mg/kg to about 3 mg/kg.

Disclosed herein are methods of treating or preventing prostate cancer in a subject, the method comprising administering to the subject:

1 mg/kg to 3 mg/kg of an anti-CTLA4 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;

1 mg/kg to 3 mg/kg of an anti-CTLA4 antibody and a vaccine comprising 1×10⁸ infectious units (IFU) of an MVA virus at about week 9, wherein the MVA virus comprises a nucleotide sequence encoding the amino acid sequence of SEQ ID NO: 3;

1 mg/kg to 3 mg/kg of an anti-CTLA4 antibody and a vaccine comprising 1×10¹¹ VP of the GAd20 virus at about week 15 and about week 18;

1 mg/kg to 3 mg/kg of an anti-CTLA4 antibody and a vaccine comprising 1×10⁸ IFU of the MVA virus at about week 24; and

1 mg/kg to 3 mg/kg of an anti-CTLA4 antibody and a vaccine comprising 1×10⁸ IFU of the MVA virus at about week 36, about week 48, and about week 60.

Disclosed herein are methods of treating or preventing prostate cancer in a subject, the method comprising administering to the subject:

1 mg/kg to 3 mg/kg of an anti-PD1 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;

1 mg/kg to 3 mg/kg of an anti-PD1 antibody and a vaccine comprising 1×10⁸ infectious units (IFU) of an MVA virus at about week 9, wherein the MVA virus comprises a nucleotide sequence encoding the amino acid sequence of SEQ ID NO: 3;

1 mg/kg to 3 mg/kg of an anti-PD1 antibody and a vaccine comprising 1×10¹¹ VP of the GAd20 virus at about week 15 and about week 18;

1 mg/kg to 3 mg/kg of an anti-PD1 antibody and a vaccine comprising 1×10⁸ IFU of the MVA virus at about week 24; and

1 mg/kg to 3 mg/kg of an anti-PD1 antibody and a vaccine comprising 1×10⁸ IFU of the MVA virus at about week 36, about week 48, and about week 60.

Disclosed herein are methods of treating or preventing prostate cancer in a subject, the method comprising administering to the subject:

1 mg/kg to 3 mg/kg of an anti-CTLA4 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;

1 mg/kg to 3 mg/kg of an anti-CTLA4 antibody and a vaccine comprising 1×10⁸ infectious units (IFU) of an MVA virus at about week 9, wherein the MVA virus comprises a nucleotide sequence encoding the amino acid sequence of SEQ ID NO: 3;

1 mg/kg to 3 mg/kg of an anti-CTLA4 antibody and a vaccine comprising 1×10¹¹ VP of the GAd20 virus at about week 15;

1 mg/kg to 3 mg/kg of an anti-CTLA4 antibody and a vaccine comprising 1×10⁸ IFU of the MVA virus at about week 24; and

1 mg/kg to 3 mg/kg of an anti-CTLA4 antibody and a vaccine comprising 1×10⁸ IFU of the MVA virus at about week 36, about week 48, and about week 60.

Disclosed herein are methods of treating or preventing prostate cancer in a subject, the method comprising administering to the subject:

1 mg/kg to 3 mg/kg of an anti-PD1 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;

1 mg/kg to 3 mg/kg of an anti-PD1 antibody and a vaccine comprising 1×10⁸ infectious units (IFU) of an MVA virus at about week 9, wherein the MVA virus comprises a nucleotide sequence encoding the amino acid sequence of SEQ ID NO: 3;

1 mg/kg to 3 mg/kg of an anti-PD1 antibody and a vaccine comprising 1×10¹¹ VP of the GAd20 virus at about week 15;

1 mg/kg to 3 mg/kg of an anti-PD1 antibody and a vaccine comprising 1×10⁸ IFU of the MVA virus at about week 24; and

1 mg/kg to 3 mg/kg of an anti-PD1 antibody and a vaccine comprising 1×10⁸ IFU of the MVA virus at about week 36, about week 48, and about week 60.

Disclosed herein are methods of treating or preventing prostate cancer in a subject, the method comprising administering to the subject:

1 mg/kg to 3 mg/kg of an anti-CTLA4 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;

1 mg/kg to 3 mg/kg of an anti-CTLA4 antibody and a vaccine comprising 1×10⁸ infectious units (IFU) of an MVA virus at about week 9, wherein the MVA virus comprises a nucleotide sequence encoding the amino acid sequence of SEQ ID NO: 3;

1 mg/kg to 3 mg/kg of an anti-CTLA4 antibody and a vaccine comprising 1×10⁸ IFU of the MVA virus at about week 15, about week 18, and about week 24; and 1 mg/kg to 3 mg/kg of an anti-CTLA4 antibody and a vaccine comprising 1×10⁸ IFU of the MVA virus at about week 36, about week 48, and about week 60.

Disclosed herein are methods of treating or preventing prostate cancer in a subject, the method comprising administering to the subject:

1 mg/kg to 3 mg/kg of an anti-PD1 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;

1 mg/kg to 3 mg/kg of an anti-PD1 antibody and a vaccine comprising 1×10⁸ infectious units (IFU) of an MVA virus at about week 9, wherein the MVA virus comprises a nucleotide sequence encoding the amino acid sequence of SEQ ID NO: 3;

1 mg/kg to 3 mg/kg of an anti-PD1 antibody and a vaccine comprising 1×10⁸ IFU of the MVA virus at about week 15, about week 18, and about week 24; and

1 mg/kg to 3 mg/kg of an anti-PD1 antibody and a vaccine comprising 1×10⁸ IFU of the MVA virus at about week 36, about week 48, and about week 60.

Disclosed herein are methods of treating or preventing prostate cancer in a subject, the method comprising administering to the subject:

1 mg/kg to 3 mg/kg of an anti-CTLA4 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;

1 mg/kg to 3 mg/kg of an anti-CTLA4 antibody and a vaccine comprising 1×10⁸ infectious units (IFU) of an MVA virus at about week 9, wherein the MVA virus comprises a nucleotide sequence encoding the amino acid sequence of SEQ ID NO: 3;

1 mg/kg to 3 mg/kg of an anti-CTLA4 antibody and a vaccine comprising 1×10⁸ IFU of the MVA virus at about week 15 and about week 24; and

1 mg/kg to 3 mg/kg of an anti-CTLA4 antibody and a vaccine comprising 1×10⁸ IFU of the MVA virus at about week 36, about week 48, and about week 60.

Disclosed herein are methods of treating or preventing prostate cancer in a subject, the method comprising administering to the subject:

1 mg/kg to 3 mg/kg of an anti-PD1 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;

1 mg/kg to 3 mg/kg of an anti-PD1 antibody and a vaccine comprising 1×10⁸ infectious units (IFU) of an MVA virus at about week 9, wherein the MVA virus comprises a nucleotide sequence encoding the amino acid sequence of SEQ ID NO: 3;

1 mg/kg to 3 mg/kg of an anti-PD1 antibody and a vaccine comprising 1×10⁸ IFU of the MVA virus at about week 15 and about week 24; and

1 mg/kg to 3 mg/kg of an anti-PD1 antibody and a vaccine comprising 1×10⁸ IFU of the MVA virus at about week 36, about week 48, and about week 60.

Each of the one or more GAd20 viruses can comprise the nucleotide sequence of SEQ ID NO: 2. In some embodiments, the nucleotide sequence further comprises an N-terminal TCE, a C-terminal His tag, or both. The TCE can comprise the amino acid sequence of SEQ ID NO: 5. The TCE can be encoded by the nucleotide sequence of SEQ ID NO: 6. The His tag can comprise the amino acid sequence of SEQ ID NO: 7. The His tag can be encoded by the nucleotide sequence of SEQ ID NO: 8. The GAd20 virus can comprise a nucleotide sequence encoding the amino acid sequence of SEQ ID NO: 9. In some embodiments, the GAd20 virus can comprise the nucleotide sequence of SEQ ID NO: 10.

Each of the one or more MVA viruses can comprise the nucleotide sequence of SEQ ID NO: 4. In some embodiments, the nucleotide sequence further comprises an N-terminal TCE, a C-terminal His tag, or both. The TCE can comprise the amino acid sequence of SEQ ID NO: 5. The TCE can be encoded by the nucleotide sequence of SEQ ID NO: 6. The His tag can comprise the amino acid sequence of SEQ ID NO: 7. The His tag can be encoded by the nucleotide sequence of SEQ ID NO: 8. The MVA virus can comprise a nucleotide sequence encoding the amino acid sequence of SEQ ID NO: 11. In some embodiments, the MVA virus can comprise the nucleotide sequence of SEQ ID NO: 12.

The methods can comprise screening the subject for the presence of one or more prostate cancer neoantigens encoding an amino acid sequence of SEQ ID NO: 13, 15, 17, 19, 21, 23,25,27,29,31,33,35,37,39,41,43,45,47,49,51,53,55,57,59,61,63,65,67,69,71,73, 75, 77, 79, 81, 83, 85, 87, 89, 91, or 93 prior to administering the one or more vaccines. In some embodiments, the methods comprise screening for the presence of one or more neoantigens comprising the nucleotide sequence of SEQ ID NO: 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, or 94 prior to administering the vaccine. For example, the disclosed methods of treating or preventing prostate cancer in a subject can comprise:

screening for the presence of one or more prostate cancer neoantigens encoding an amino acid sequence of SEQ ID NO: 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, or 93; and

if the one or more neoantigens are detected, administering to the subject a treatment regimen comprising
two or more vaccines comprising a great ape adenovirus serotype 20 (GAd20) virus that, in turn, comprises a nucleotide sequence encoding the amino acid sequence of SEQ ID NO: 1 and
one or more vaccines comprising a Modified Vaccinia Ankara (MVA) virus that, in turn, comprises a nucleotide sequence encoding the amino acid sequence of SEQ ID NO: 3

to thereby treat or prevent the prostate cancer.

The screening can comprise analyzing the presence of the neoantigen protein or analyzing the presence of a nucleic acid sequence encoding the neoantigen protein. Exemplary screening techniques include, for example, genotyping, PCR, and protein analysis.

### EXAMPLES

The following examples are provided to further describe some of the embodiments disclosed herein. The examples are intended to illustrate, not to limit, the disclosed embodiments.

### Immunogenicity Testing of Gad20/MVA in Combination with a αCTLA4 and/or αPD1 Antibodies in Cynomolgus Monkeys

The primary aim of the study was to determine if GAd20/GAd20/MVA dosing can generate a prostate neoantigen-specific T cell response in non-human primates (NHPs), which can be further enhanced using anti-CTLA4 antibodies (αCTLA4) and anti-PD-1 antibodies (αPD-1). A secondary aim was to evaluate if two sequential prime immunizations with GAd20 at week 0 and 4 increased the number of responding animals by week 6.

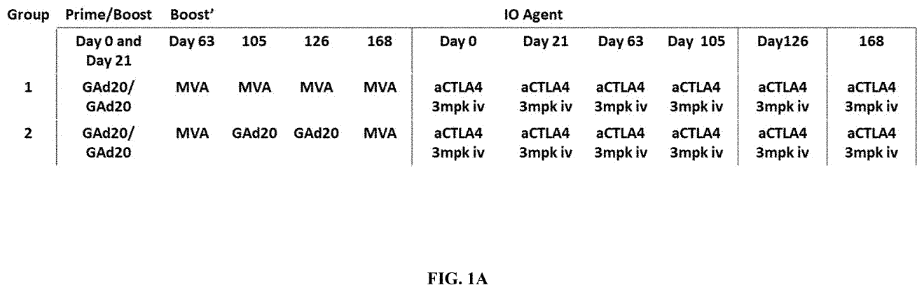

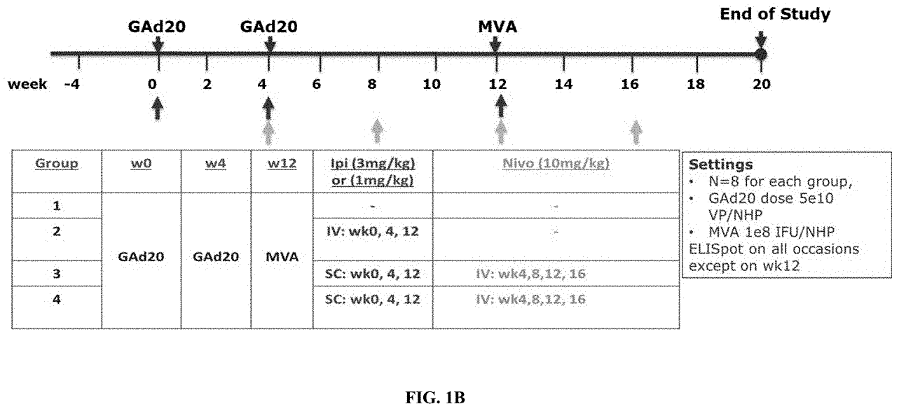

The dosing sequence used in this study is provided in FIG. 1B. Briefly, animals received 5×10¹⁰ VP of GAd20 comprising a nucleotide sequence of SEQ ID NO: 10, which encodes the transgene of SEQ ID NO: 9, at week 0 and week 4, and 1 ×10⁸ IFU of MVA comprising a nucleotide sequence of SEQ ID NO: 12, which encodes the transgene of SEQ ID NO: 11, at week 12 (Groups 1-4). Group 2 received 3 mg/kg IV of an anti-CTLA4 antibody (Ipi) at week 0, 4, and week 12. Group 3 received 3 mg/kg SC of an anti-CTLA4 antibody (Ipi) at week 0, 4, and week 12 and 10 mg/kg IV of an anti-PD-1 antibody at week 4, week 8, week 12, and week 16. Group 4 received 3 mg/kg SC of an anti-CTLA4 antibody (Ipi) at week 0 and 4 and 1 mg/kg SC of an anti-CTLA4 antibody (Ipi) on week 12 and 10 mg/kg IV of an anti-PD-1 antibody at week 4, week 8, week 12, and week 16.

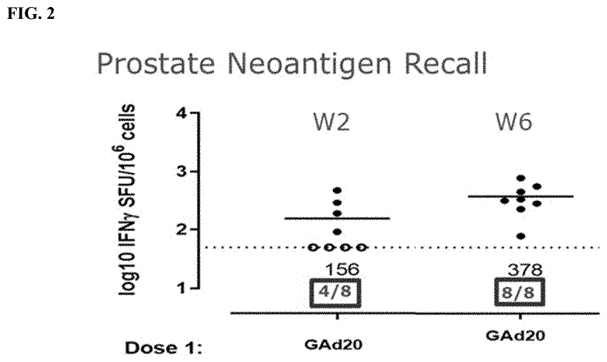

PBMCs from NHPs were tested using an overnight peptide recall IFNγ ELISpot assay. As shown in FIG. 2, the GAd20 vaccine is immunogenic in NHP (4 of 8 responders in group 1 at week 2). A GAd20 double-prime increased the number of responding animals (8 of 8 responders in group 1 at week 6) and increased the frequency of antigen specific T cell responses. These results confirm that GAd20 is immunogenic and a double-prime enhances vaccine-specific T cell response at W6.

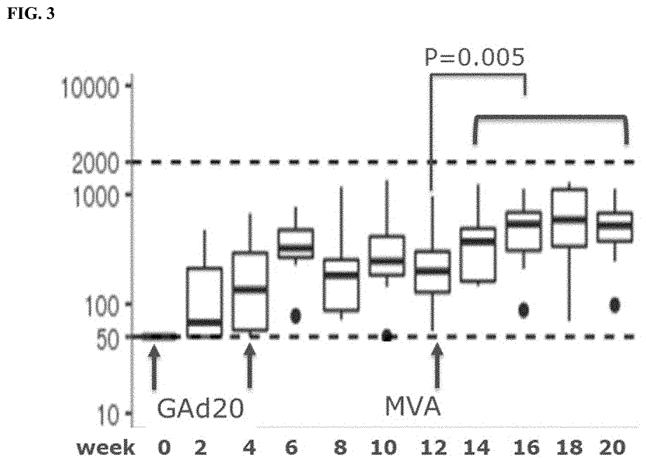

As shown in FIG. 3, the MVA vaccine increased antigen specific T cell response 2.4× at week 18 compared to week 12. The mean of all timepoints post week 12 was significantly elevated (P=0.005) compared to the response at week 12. These results confirm that MVA is immunogenic and significantly boosts a 2× GAd20 primed antigen specific T cell response.

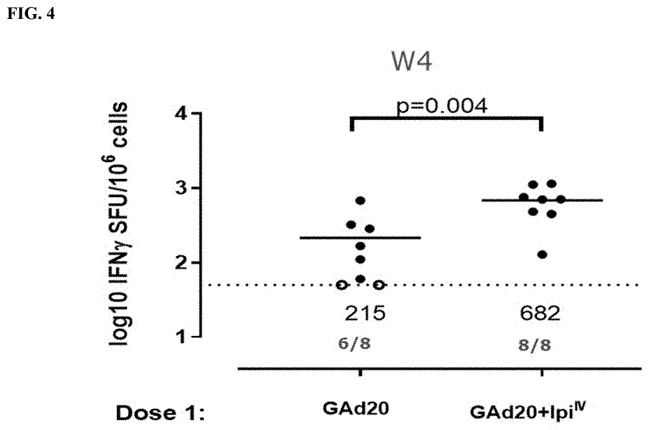

As illustrated in FIG. 4, co-administration of an αCTLA4 significantly increased the number of responding animals and the magnitude of antigen specific T cell response 2 weeks after the first GAd20 immunization. With GAd20 only, there was a mean of 215 SFU/1e6 PBMCs at week 4 with only 6 of 8 animals responding (group 1). With the concomitant dosing of αCTLA4, the mean increased to 682 SFU/1e6 PBMCs and all animals responded (group 2).

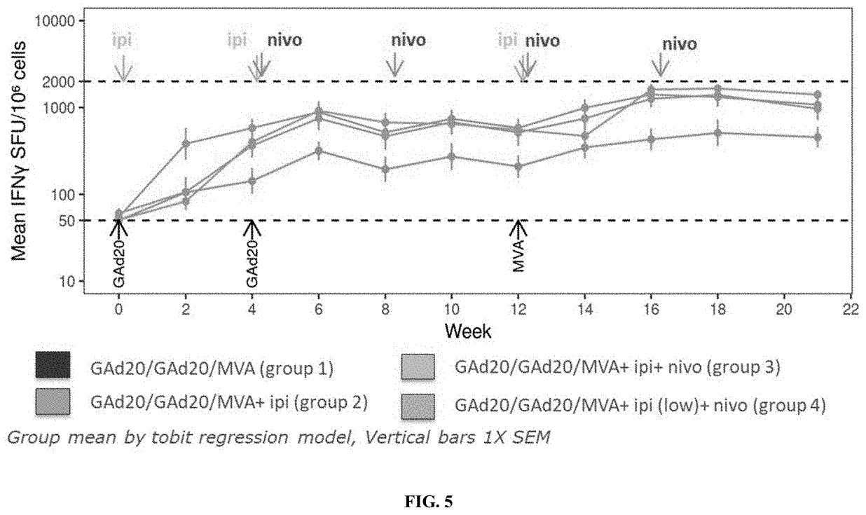

αCTLA4 was shown to increase the magnitude of the prostate neo-antigen specific T cell response for the duration of the study (FIG. 5). As shown in FIG. 5, MVA boosted the immune response by week 16; αCTLA4 increased the magnitude of the immune response over the entire duration of the study; and αPD-1 (groups 3 & 4) did not significantly reduce the antigen-specific immune responses. These results confirm that (1) the co-administration of αCTLA4 improves the magnitude of T cell response across all timepoints compared to vaccination alone and (2) the combination anti-PD-1 and αCTLA4 with GAd20/MVA heterologous multi-dose vaccination also improves the magnitude of antigen specific T cell responses across all timepoints compared to vaccination alone.

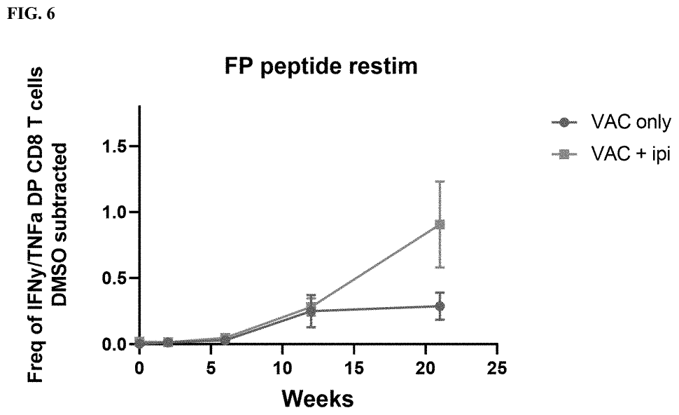

NHP PBMCs were tested in an overnight peptide recall Intracellular Cytokine Staining (ICS) assay. As shown in FIG. 6, NHPs dosed with vaccine+αCTLA4 generated more neo-antigens specific polyfunctional (IFNγ/TNFα double positive) CD8+ T cells than NHPs dosed with vaccine alone. These results indicate that co-administration of αCTLA4 increases the quality of antigen specific T cells as evidence of increased frequency of polyfunctional CD8+ T cells.

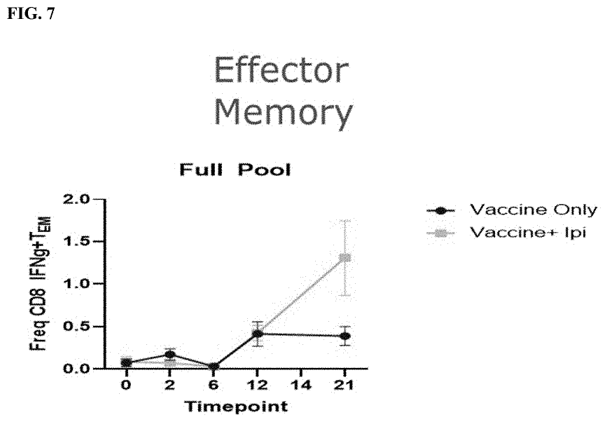

As shown in FIG. 7, effector memory neo-antigen specific CD8+ T cells are increased with concomitant αCTLA4 compared to vaccine alone at week 21.

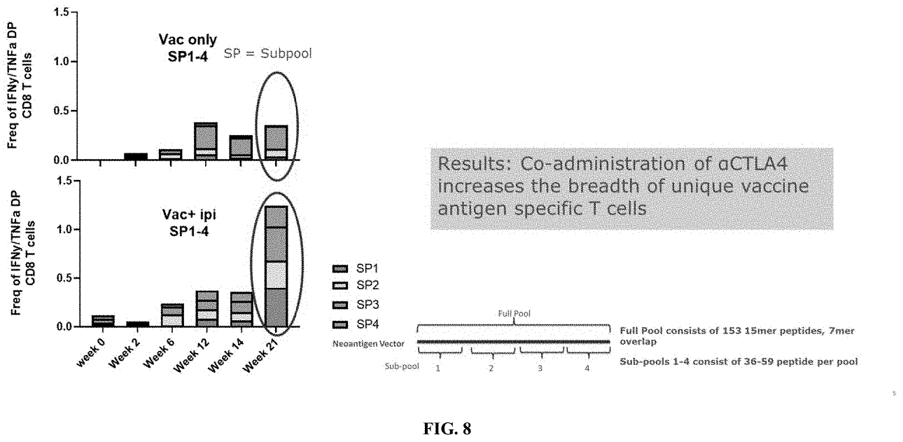

NHP PBMCs were tested in an ICS assay utilizing 4 sub pools of peptides, each consisting of approximately ¼ of the total neo-antigens in the vaccine. As shown in FIG. 8, the vaccine alone generated predominantly Subpool 3 responses while animals that received both aCTLA4 and vaccine generated high frequency of T cell responses specific to all 4 subpools. Thus, the co-administration of αCTLA4 expands the breadth of unique vaccine antigen specific T cells.

### Conclusions

GAd20 can effectively prime a prostate neo-antigen immune response in NHP, and the use of a “double prime” of GAd20 increased the number of responding animals.
MVA effectively boosted animals that received 2X GAd20 immunizations.
The addition of αCTLA4 to the vaccine generated a durable immune response of increased magnitude in both the overall immune response as measured by ELISpot and in the frequency of antigen-specific polyfunctional CD8 T cell responses.
αCTLA4 increased the frequency of both neo-antigen specific effector CD8+ T cells and can increase the breadth of neo-antigen specific immune responses.
The addition of αPD-1 does not significantly reduce the neo-antigen specific immune responses.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

TABLE 2
Sequences

| SEQ ID | |
|---|---|
| NO: | Sequence |
| 1 | QNLQNGGGSRSSATLPGRRRRRWLRRRRQPISVAPAGPPRRPNQKPNPPGGARCVI |
| GAd20 | MRPTWPGTSAFTKRSFAVTERIIDYWAQKEKGSSSFLRPSCDYWAQKEKISIPRTH |
| Transgene | LCLVLGVLSGHSGSRLYEAGMTLGGKILFFLFLLLPLSPFSLIFTEISCCTLSSEE |
| amino | NEYLPRPEWQLQVPFRELKNVSVLEGLRQGRLGGPCSCHCPRPSQARLTPVDVAGP |
| acid | FLCLGDPGLFPPVKSSITGGKSTCSAPGPQSLPSTPFSTYPQWVILITELGMECTL |
| | GQVGAPSPRREEDGWRGGHSRFKADVPAPQGPCWGGQPGSAPSSAPPEQSLLDWKF |
| | EMSYTVGGPPPHVHARPRHWKTDRDGHSYTSKVNCLLLQDGFHGCVSITGAAGRRN |
| | LSIFLFLMLCKLEFHACKIQNKNCPDFKKFDGPCGERGGGRTARALWARGDSVLTP |
| | ALDPQTPVRAPSLTRAAAAVHYKLIQQPISLFSITDRLHKTFSQLPSVHLCSITFQ |
| | WGHPPIFCSTNDICVTANFCISVTFLKPCFLLHEASASQCHLFLQPQVGTPPPHTA |
| | SARAPSGPPHPHESCPAGRRPARAAQTCARRQHGLPGCEEAGTARVPSLHLHLHQA |
| | ALGAGRGRGWGEACAQVPPSRGVLRFLDLKVRYLHSQWQHYHRSGEAAGTPLWRPT |
| | RNVPFRELKNQRTAQGAPGIHHAASPVAANLCDPARHAQHTRIPCGAGQVRAGRGP |
| | EAGGGVLQPQRPAPEKPGCPCRRGQPRLHTVKMWRAVAMMVPDRQVHYDFGLGVPG |
| | DSTRRAVRRMNTFYEAGMTLGEKFRVGNCKHLKMTRPNSKMALNSEALSVVSECGA |
| | SACDVSLIAMDSAFVQGKDWGVKKFIRRDFYAYKDFLWCFPFSLVFLQEIQICCHV |
| | SCLCCICCSTRICLGCLLELFLSRALRALHVLWNGFQLHCQTEYNQKLQVNQFSES |
| | KSLYHREKQLIAMDSAICEERGAAGSLISCETMPAILKLQKNCLLSLRTALTHNQD |
| | FSIYRLCCKRGSLCHASQARSPAFPKPVRPLPAPITRITPQLGGQSDSSQPLLTTG |
| | RPQGWQDQALRHTQQASPASCATITIPIHSAALGDHSGDPGPAWDTCPPLPLTTLI |
| | PRAPPPYGDSTARSWPSRCGPLGGNTTLQQLGEASQAPSGSLIPLRLPLLWEVRGQ |
| | PDSFAALHSSLNELGEIARELHQFAFDLLIKSHFVQGKDWGLKKFIRRDFWGMELA |
| | ASRRFSWDHHSAGGPPRVPSVRSGAAQVQPKDPLPLRTLAGCLARTAHLRPGAESL |
| | PQPQLHCTLWFQSSELSPTGAPWPSRRPTWRGTTVSPRTATSSARTCCGTKWPSSQ |
| | EAALGLGSGLLRFSCGTAAIRKMHFSLKEHPPPPCPPEAFQRAAGEGGPGRGGARR |
| | GARVLQSPFCRAGAGEWLGHQSLRHVVGYGHLDTSGSSSSSSWPNSKMALNSLNSI |
| | DDAQLTRIAPPRSHCCFWEVNAP |
| 2 | CAGAACCTGCAGAACGGCGGAGGCTCTAGAAGCTCTGCTACACTTCCTGGCAGGCG |
| GAd20 | GCGGAGAAGATGGCTGAGAAGAAGGCGGCAGCCTATCTCTGTGGCTCCTGCTGGAC |
| Transgene | CTCCTAGACGGCCCAACCAGAAGCCTAATCCTCCTGGCGGAGCCAGATGCGTGATC |
| nucleic | ATGAGGCCTACATGGCCTGGCACCAGCGCCTTCACCAAGAGAAGCTTTGCCGTGAC |
| acid | CGAGCGGATCATCGACTATTGGGCTCAAAAAGAGAAGGGCAGCAGCAGCTTCCTGC |
| | GGCCTAGCTGTGATTATTGGGCCCAGAAAGAAAAGATCAGCATCCCCAGAACACAC |
| | CTGTGCCTGGTGCTGGGAGTGCTGTCTGGACACTCTGGCAGCAGACTGTATGAGGC |
| | CGGCATGACACTCGGCGGCAAGATCCTGTTCTTCCTGTTCCTGCTGCTCCCTCTGA |
| | GCCCCTTCAGCCTGATCTTCACCGAGATCAGCTGCTGCACCCTGAGCAGCGAGGAA |
| | AACGAGTACCTGCCTAGACCTGAGTGGCAGCTGCAGGTCCCCTTCAGAGAGCTGAA |
| | GAACGTTTCCGTGCTGGAAGGCCTGAGACAGGGCAGACTTGGCGGCCCTTGTAGCT |
| | GTCACTGCCCCAGACCTAGTCAGGCCAGACTGACACCTGTGGATGTGGCCGGACCT |
| | TTCCTGTGTCTGGGAGATCCTGGCCTGTTTCCACCTGTGAAGTCCAGCATCACAGG |
| | CGGCAAGTCCACATGTTCTGCCCCTGGACCTCAGAGCCTGCCTAGCACACCCTTCA |
| | GCACATACCCTCAGTGGGTCATCCTGATCACCGAACTCGGCATGGAATGCACCCTG |
| | GGACAAGTGGGAGCCCCATCTCCTAGAAGAGAAGAGGATGGCTGGCGCGGAGGCCA |
| | CTCTAGATTCAAAGCTGATGTGCCCGCTCCTCAGGGCCCTTGTTGGGGAGGACAAC |
| | CTGGATCTGCCCCATCTTCTGCCCCACCTGAACAGTCCCTGCTGGATTGGAAGTTC |
| | GAGATGAGCTACACCGTCGGCGGACCTCCACCTCATGTTCATGCCAGACCTCGGCA |
| | CTGGAAAACCGACAGAGATGGCCACAGCTACACCAGCAAAGTGAACTGCCTCCTGC |
| | TGCAGGATGGCTTCCACGGCTGTGTGTCTATTACTGGCGCCGCTGGCAGACGGAAC |
| | CTGAGCATCTTTCTGTTTCTGATGCTGTGCAAGCTCGAGTTCCACGCCTGCAAGAT |
| | CCAGAACAAGAACTGCCCCGACTTCAAGAAGTTCGACGGCCCTTGCGGAGAAAGAG |
| | GCGGAGGCAGAACAGCTAGAGCCCTTTGGGCTAGAGGCGACAGCGTTCTGACACCA |
| | GCTCTGGACCCTCAGACACCTGTTAGGGCCCCTAGCCTGACAAGAGCTGCCGCCGC |
| | TGTGCACTACAAGCTGATCCAGCAGCCAATCAGCCTGTTCAGCATCACCGACCGGC |
| | TGCACAAGACATTCAGCCAGCTGCCAAGCGTGCACCTGTGCTCCATCACCTTCCAG |
| | TGGGGACACCCTCCTATCTTTTGCTCCACCAACGACATCTGCGTGACCGCCAACTT |
| | CTGTATCAGCGTGACCTTCCTGAAGCCTTGCTTTCTGCTGCACGAGGCCAGCGCCT |
| | CTCAGTGCCACTTGTTTCTGCAGCCCCAAGTGGGCACACCTCCTCCACATACAGCC |
| | TCTGCTAGAGCACCTAGCGGCCCTCCACATCCTCACGAATCTTGTCCTGCCGGAAG |
| | AAGGCCTGCCAGAGCCGCTCAAACATGTGCCAGACGACAGCACGGACTGCCTGGAT |
| | GTGAAGAGGCTGGAACAGCCAGAGTGCCTAGCCTGCACCTCCATCTGCATCAGGCT |
| | GCTCTTGGAGCCGGAAGAGGTAGAGGATGGGGCGAAGCTTGTGCTCAGGTGCCACC |
| | TTCTAGAGGCGTGCTGAGATTCCTGGACCTGAAAGTGCGCTACCTGCACAGCCAGT |
| | GGCAGCACTATCACAGATCTGGCGAAGCCGCCGGAACACCCCTTTGGAGGCCAACA |
| | AGAAACGTGCCCTTCCGGGAACTGAAGAACCAGAGAACAGCTCAGGGCGCTCCTGG |
| | AATCCACCATGCTGCTTCTCCAGTGGCCGCCAACCTGTGTGATCCTGCCAGACATG |
| | CCCAGCACACCAGGATTCCTTGTGGCGCTGGACAAGTGCGCGCTGGAAGAGGACCT |
| | GAAGCAGGCGGAGGTGTTCTGCAACCTCAAAGACCCGCTCCTGAGAAGCCTGGCTG |
| | CCCTTGCAGAAGAGGACAGCCTAGACTGCACACCGTGAAAATGTGGCGAGCCGTGG |
| | CCATGATGGTGCCCGATAGACAGGTCCACTACGACTTTGGACTGGGCGTGCCAGGC |
| | GATAGCACTCGGAGAGCCGTCAGACGGATGAACACCTTTTACGAAGCCGGGATGAC |
| | CCTGGGCGAGAAGTTCAGAGTGGGCAACTGCAAGCACCTGAAGATGACCCGGCCTA |
| | ACAGCAAGATGGCCCTGAATAGCGAGGCCCTGTCTGTGGTGTCTGAATGTGGCGCC |
| | TCTGCCTGTGACGTGTCCCTGATCGCTATGGACTCCGCCTTTGTGCAGGGCAAAGA |
| | CTGGGGCGTGAAGAAGTTCATCCGGCGGGACTTCTACGCCTACAAGGACTTCCTGT |
| | GGTGCTTCCCCTTCTCTCTGGTGTTCCTGCAAGAGATCCAGATCTGCTGTCATGTG |
| | TCCTGCCTGTGCTGCATCTGCTGTAGCACCAGAATCTGCCTGGGCTGTCTGCTGGA |
| | ACTGTTCCTGAGCAGAGCCCTGAGAGCACTGCACGTGCTGTGGAACGGATTCCAGC |
| | TGCACTGCCAGACCGAGTACAACCAGAAACTGCAAGTGAACCAGTTCAGCGAGAGC |
| | AAGAGCCTGTACCACCGGGAAAAGCAGCTCATTGCCATGGACAGCGCCATCTGCGA |
| | AGAGAGAGGCGCCGCAGGATCTCTGATCTCCTGCGAAACAATGCCCGCCATCCTGA |
| | AGCTGCAGAAGAATTGCCTCCTAAGCCTGCGAACCGCTCTGACACACAACCAGGAC |
| | TTCAGCATCTACAGACTGTGTTGCAAGCGGGGCTCCCTGTGCCATGCAAGCCAAGC |
| | TAGAAGCCCCGCCTTTCCTAAACCTGTGCGACCTCTGCCAGCTCCAATCACCAGAA |
| | TTACCCCTCAGCTCGGCGGCCAGAGCGATTCATCTCAACCTCTGCTGACCACCGGC |
| | AGACCTCAAGGCTGGCAAGACCAAGCTCTGAGACACACCCAGCAGGCTAGCCCTGC |
| | CTCTTGTGCCACCATCACAATCCCCATCCACTCTGCCGCTCTGGGCGATCATTCTG |
| | GCGATCCTGGACCAGCCTGGGACACATGTCCTCCACTGCCACTCACAACACTGATC |
| | CCTAGGGCTCCTCCACCTTACGGCGATTCTACCGCTAGAAGCTGGCCCAGCAGATG |
| | TGGACCACTCGGAGGCAACACAACCCTCCAGCAACTGGGAGAAGCCTCTCAGGCTC |
| | CTAGCGGCTCTCTGATCCCTCTCAGACTGCCTCTCCTGTGGGAAGTTCGGGGCCAG |
| | CCTGATTCTTTTGCCGCACTGCACAGCTCCCTGAACGAGCTGGGAGAGATCGCTAG |
| | AGAGCTGCACCAGTTCGCCTTCGACCTGCTGATCAAGAGCCACTTCGTGCAAGGCA |
| | AGGATTGGGGCCTCAAAAAGTTTATCCGCAGAGACTTCTGGGGCATGGAACTGGCC |
| | GCCAGCAGAAGATTCAGCTGGGATCATCATAGCGCAGGCGGCCCACCTAGAGTGCC |
| | ATCTGTTAGAAGCGGAGCTGCCCAGGTGCAGCCTAAAGATCCTCTGCCACTGAGAA |
| | CACTGGCCGGCTGCCTTGCTAGAACAGCCCATCTTAGACCTGGCGCCGAGTCTCTG |
| | CCTCAGCCACAACTGCACTGTACCCTGTGGTTCCAGTCCAGCGAGCTGTCTCCTAC |
| | TGGTGCCCCTTGGCCATCTAGACGCCCTACTTGGAGAGGCACCACCGTGTCACCAA |
| | GAACCGCCACAAGCAGCGCCAGAACCTGTTGTGGCACAAAGTGGCCCTCCAGCCAA |
| | GAAGCCGCTCTCGGACTTGGAAGCGGACTGCTGAGGTTCTCTTGTGGAACCGCCGC |
| | CATTCGGAAGATGCACTTTAGCCTGAAAGAACACCCTCCACCACCTTGTCCTCCAG |
| | AGGCTTTCCAAAGAGCTGCTGGCGAAGGCGGACCTGGTAGAGGTGGTGCTAGAAGA |
| | GGTGCTAGGGTGCTGCAGAGCCCATTCTGTAGAGCAGGCGCAGGCGAATGGCTGGG |
| | CCATCAGAGTCTGAGACATGTCGTCGGCTACGGCCACCTGGATACAAGCGGAAGCA |
| | GCTCTAGCTCCAGCTGGCCTAACTCAAAAATGGCTCTGAACAGCCTGAACTCCATC |
| | GACGACGCCCAGCTGACAAGAATCGCCCCTCCTAGATCTCACTGCTGCTTTTGGGA |
| | AGTGAACGCCCCA |
| 3 | QNLQNGGGSRSSATLPGRRRRRWLRRRRQPISVAPAGPPRRPNQKPNPPGGARCVI |
| MVA | MRPTWPGTSAFTGMECTLGQVGAPSPRREEDGWRGGHSRFKADVPAPQGPCWGGQP |
| Transgene | GSAPSSAPPEQSLLDDYWAQKEKISIPRTHLCWKFEMSYTVGGPPPHVHARPRHWK |
| amino | TDRDYWAQKEKGSSSFLRPSCVPFRELKNVSVLEGLRQGRLGGPCSCHCPRPSQAR |
| acid | LTPVDVAGPFLCLGDPGLFPPVKSSITEISCCTLSSEENEYLPRPEWQLQYEAGMT |
| | LGGKILFFLFLLLPLSPFSLIFLVLGVLSGHSGSRLKRSFAVTERIITGGKSTCSA |
| | PGPQSLPSTPFSTYPQWVILITELDGHSYTSKVNCLLLQDGFHGCVSITGAAGRRN |
| | LSIFLFLMLCKLEFHACQWQHYHRSGEAAGTPLWRPTRNVAMMVPDRQVHYDFGLK |
| | IQNKNCPDVLRFLDLKVRYLHSVPFRELKNQRTAQGAPGIHHAASPVAANLCDPAR |
| | HAQHTRIPCGAGQVRAGRGPEAGGGVLQPQRPAPEKPGCPCRRGQPRLHTVKMWRA |
| | CHLFLQPQVGTPPPHTASARAPSGPPHPHESCPAGRRPARAAQTCARRQHGLPGCE |
| | EAGTARVPSLHLHLHQAALGAGRGRGWGEACAQVPPSRGHYKLIQQPISLFSITDR |
| | LHKTFSQLPSVHLCSITFQWGHPPIFCSTNDICVTANFCISVTFLKPCFLLHEASA |
| | SQFKKFDGPCGERGGGRTARALWARGDSVLTPALDPQTPVRAPSLTRAAAAVGVPG |
| | DSTRRAVRRMNTFCGASACDVSLIAMDSACEERGAAGSLISCESLYHREKQLIAMD |
| | SAIFVQGKDWGVKKFIRRDFTMPAILKLQKNCLLSLNSKMALNSEALSVVSEYEAG |
| | MTLGEKFRVGNCKHLKMTRPTEYNQKLQVNQFSESKRTALTHNQDFSIYRLCCKRG |
| | SLCHASQARSPAFPKPVRPLPAPITRITPQLGGQSDSSQPLLTTGRPQGWQDQALR |
| | HTQQASPASCATITIPIHSAALGDHSGDPGPAWDTCPPLPLTTLIPRAPPPYGDST |
| | ARSWPSRCGPLGYAYKDFLWCFPFSLVFLQEIQICCHVSCLCCICCSTRICLGCLL |
| | ELFLSRALRALHVLWNGFQLHCQGNTTLQQLGEASQAPSGSLIPLRLPLLWEVRGN |
| | SKMALNSLNSIDDAQLTRIAPPRSHCCFWEVNAPFVQGKDWGLKKFIRRDFEAFQR |
| | AAGEGGPGRGGARRGARVLQSPFCRAGAGEWLGHQSLRWGMELAASRRFSWDHHSA |
| | GGPPRVPSVRSGAAQVQPKDPLPLRTLAGCLARTAHLRPGAESLPQPQLHCTIARE |
| | LHQFAFDLLIKSHKMHFSLKEHPPPPCPPHVVGYGHLDTSGSSSSSSWPQPDSFAA |
| | LHSSLNELGELWFQSSELSPTGAPWPSRRPTWRGTTVSPRTATSSARTCCGTKWPS |
| | SQEAALGLGSGLLRFSCGTAAIR |
| 4 | CAGAACCTGCAGAACGGCGGAGGCTCTAGAAGCTCTGCTACACTTCCTGGCAGGCG |
| MVA | GCGGAGAAGATGGCTGAGAAGAAGGCGGCAGCCTATCTCTGTGGCTCCTGCTGGAC |
| Transgene | CTCCTAGACGGCCCAACCAGAAGCCTAATCCTCCTGGCGGAGCCAGATGCGTGATC |
| nucleic | ATGAGGCCTACATGGCCTGGCACCAGCGCCTTTACCGGCATGGAATGTACACTGGG |
| acid | CCAAGTGGGAGCCCCATCTCCTAGAAGAGAAGAGGATGGCTGGCGCGGAGGCCACT |
| | CTAGATTCAAAGCTGATGTGCCCGCTCCTCAGGGCCCTTGTTGGGGAGGACAACCT |
| | GGATCTGCCCCATCTTCTGCCCCACCTGAACAGAGCCTGCTGGATGACTATTGGGC |
| | TCAAAAAGAGAAGATCAGCATCCCCAGAACACACCTGTGCTGGAAGTTCGAGATGA |
| | GCTACACCGTTGGCGGCCCTCCACCACATGTTCACGCCAGACCTAGACACTGGAAA |
| | ACCGACAGAGATTATTGGGCCCAGAAAGAAAAGGGCAGCAGCAGCTTCCTGCGGCC |
| | TAGCTGTGTGCCCTTCCGGGAACTGAAGAACGTGTCCGTTCTGGAAGGCCTGAGGC |
| | AGGGCAGACTTGGCGGACCTTGTAGCTGCCACTGTCCTAGACCAAGCCAGGCCAGA |
| | CTGACCCCTGTGGATGTGGCTGGCCCATTTCTGTGTCTGGGCGACCCTGGACTGTT |
| | CCCTCCAGTGAAGTCTAGCATCACCGAGATCAGCTGCTGCACCCTGAGCAGCGAGG |
| | AAAACGAGTACCTGCCTAGACCTGAATGGCAGCTGCAGTACGAGGCCGGCATGACA |
| | CTCGGAGGCAAGATCCTGTTCTTCCTGTTCCTGCTGCTCCCTCTGAGCCCCTTCAG |
| | CCTGATCTTTCTGGTGCTGGGCGTGCTGTCTGGCCACTCTGGAAGCAGACTGAAGA |
| | GAAGCTTCGCCGTGACCGAGCGGATCATCACAGGCGGCAAGAGCACATGTTCTGCC |
| | CCTGGACCTCAGTCTCTGCCCAGCACACCCTTCAGCACATACCCTCAGTGGGTCAT |
| | CCTGATCACCGAGCTGGATGGCCACAGCTACACCAGCAAAGTGAACTGCCTCCTGC |
| | TGCAGGATGGCTTCCACGGCTGTGTGTCTATTACTGGCGCCGCTGGCAGACGGAAC |
| | CTGAGCATCTTTCTGTTTCTGATGCTGTGCAAGCTCGAGTTCCACGCCTGCCAATG |
| | GCAGCACTACCACAGATCTGGCGAAGCCGCTGGAACCCCACTTTGGAGGCCTACCA |
| | GAAACGTGGCCATGATGGTGCCCGACAGACAGGTGCACTACGACTTCGGCCTGAAG |
| | ATCCAGAACAAGAACTGCCCCGACGTGCTGCGGTTCCTGGATCTCAAAGTGCGCTA |
| | CCTGCACAGCGTGCCCTTCAGAGAGCTGAAAAACCAGAGAACAGCCCAGGGCGCTC |
| | CTGGAATCCATCATGCTGCTTCTCCAGTGGCCGCCAATCTGTGCGATCCTGCCAGA |
| | CATGCCCAGCATACCAGGATTCCTTGTGGCGCTGGACAAGTGCGCGCTGGAAGAGG |
| | ACCTGAAGCTGGTGGCGGAGTTCTGCAGCCTCAAAGACCTGCTCCTGAGAAGCCTG |
| | GCTGCCCCTGTAGAAGAGGACAGCCTAGACTGCACACCGTGAAGATGTGGCGGGCC |
| | TGCCACTTGTTTCTCCAGCCACAAGTGGGCACCCCTCCACCTCATACAGCCTCTGC |
| | TAGAGCACCTAGCGGCCCACCTCATCCTCACGAATCTTGTCCTGCCGGAAGAAGGC |
| | CTGCCAGAGCCGCTCAAACATGTGCCAGACGACAGCACGGACTGCCCGGATGTGAA |
| | GAAGCCGGAACAGCCAGAGTGCCTAGCCTGCACCTTCATCTGCATCAGGCCGCTCT |
| | TGGAGCCGGAAGAGGTAGAGGATGGGGAGAAGCTTGTGCCCAGGTGCCACCTTCTA |
| | GAGGCCACTACAAGCTGATCCAGCAGCCAATCAGCCTGTTCTCCATCACCGACCGG |
| | CTGCACAAGACATTCAGCCAGCTGCCTTCCGTGCATCTGTGCAGCATCACCTTCCA |
| | GTGGGGACACCCTCCTATCTTTTGCTCCACCAACGACATCTGCGTGACCGCCAACT |
| | TCTGTATCAGCGTGACCTTCCTGAAGCCTTGCTTTCTGCTGCACGAGGCCTCCGCC |
| | AGCCAGTTTAAGAAGTTTGACGGCCCCTGCGGCGAGAGAGGCGGAGGAAGAACTGC |
| | AAGAGCCCTTTGGGCCAGAGGCGACTCTGTTCTGACACCAGCTCTGGACCCTCAGA |
| | CACCTGTTAGGGCCCCTAGCCTGACAAGAGCTGCCGCTGCTGTTGGAGTGCCTGGC |
| | GATTCTACTAGAAGGGCCGTGCGGCGGATGAACACCTTTTGTGGCGCATCTGCCTG |
| | CGACGTGTCCCTGATCGCTATGGATAGCGCCTGCGAGGAAAGAGGCGCAGCCGGAT |
| | CTCTGATCTCTTGCGAGAGCCTGTACCACCGGGAAAAGCAGCTCATTGCCATGGAC |
| | AGCGCCATCTTCGTGCAGGGCAAAGACTGGGGCGTGAAGAAGTTCATCCGGCGGGA |
| | CTTTACCATGCCTGCCATTCTGAAGCTGCAGAAGAATTGTCTTCTAAGCCTGAACA |
| | GCAAGATGGCCCTGAATAGCGAGGCCCTGTCTGTGGTGTCCGAGTATGAGGCTGGA |
| | ATGACCCTGGGCGAGAAGTTCAGAGTGGGCAACTGCAAGCACCTGAAGATGACCCG |
| | GCCTACCGAGTACAACCAGAAACTGCAAGTGAACCAGTTCAGCGAGAGCAAGCGGA |
| | CCGCTCTGACCCACAACCAGGACTTCAGCATCTACCGGCTGTGCTGCAAGAGGGGC |
| | TCTCTGTGTCATGCTAGCCAGGCTAGAAGCCCCGCCTTTCCTAAGCCTGTCAGACC |
| | TCTGCCTGCTCCTATCACCAGAATCACCCCTCAGCTCGGCGGCCAGTCTGATTCAT |
| | CTCAGCCACTGCTGACCACCGGCAGACCTCAAGGATGGCAAGACCAGGCTCTGAGA |
| | CACACACAGCAGGCTAGCCCAGCCTCTTGCGCCACCATCACAATACCAATACATTC |
| | TGCCGCTCTGGGCGATCACAGCGGAGATCCTGGACCTGCCTGGGATACTTGTCCTC |
| | CTCTGCCCCTAACTACACTGATCCCTAGGGCTCCTCCACCTTACGGCGATAGCACA |
| | GCCAGATCCTGGCCTAGCAGATGTGGCCCTCTGGGCTACGCCTACAAGGACTTCCT |
| | GTGGTGCTTCCCCTTCTCTCTGGTGTTCCTGCAAGAAATCCAGATCTGCTGTCACG |
| | TGTCCTGCCTGTGCTGTATCTGCTGTAGCACCCGGATCTGTCTGGGCTGTCTGCTG |
| | GAACTGTTCCTGAGCAGAGCCCTGAGAGCACTGCACGTGCTGTGGAACGGATTCCA |
| | GCTGCACTGCCAGGGCAACACCACACTGCAACAGCTGGGAGAAGCCTCTCAGGCCC |
| | CAAGCGGTTCTCTGATCCCTCTCAGACTGCCCCTCCTGTGGGAAGTGCGGGGCAAT |
| | TCTAAGATGGCTCTCAACAGCCTGAACTCCATCGACGACGCCCAGCTGACAAGAAT |
| | CGCCCCTCCAAGAAGCCACTGTTGCTTTTGGGAAGTGAACGCCCCTTTTGTGCAGG |
| | GTAAAGATTGGGGCCTCAAAAAGTTTATCAGACGGGACTTCGAGGCTTTCCAGAGA |
| | GCAGCTGGCGAAGGCGGACCTGGCAGAGGTGGTGCTAGAAGAGGTGCTAGAGTGCT |
| | GCAGAGCCCATTCTGTAGAGCTGGCGCTGGCGAATGGCTGGGCCACCAATCTCTTA |
| | GATGGGGAATGGAACTGGCCGCTAGCAGGCGGTTTAGCTGGGATCATCATTCTGCC |
| | GGCGGACCTCCAAGAGTGCCAAGCGTTAGAAGCGGAGCAGCCCAGGTCCAGCCTAA |
| | AGATCCACTGCCACTGAGAACACTGGCCGGCTGCCTTGCCAGAACAGCTCATCTTA |
| | GACCTGGCGCCGAAAGCCTGCCTCAACCTCAGCTGCATTGCACAATCGCCAGAGAA |
| | CTGCACCAGTTCGCCTTCGACCTGCTGATCAAGAGCCACAAGATGCACTTCTCACT |
| | GAAAGAGCACCCGCCACCGCCGTGCCCACCGCACGTTGTCGGCTATGGCCACCTGG |
| | ATACAAGCGGCTCCTCTAGCAGTAGCTCCTGGCCTCAGCCTGACAGCTTTGCTGCC |
| | CTGCATAGCTCCCTGAATGAGCTGGGCGAACTGTGGTTCCAGTCCAGCGAACTGTC |
| | TCCTACTGGCGCTCCATGGCCAAGCAGAAGGCCTACTTGGAGAGGCACCACCGTGT |
| | CTCCAAGAACCGCTACAAGCAGCGCCAGAACCTGTTGCGGCACAAAATGGCCCTCC |
| | AGCCAAGAAGCTGCCCTCGGACTTGGAAGCGGACTGCTGAGATTCAGCTGTGGCAC |
| | AGCCGCCATCAGA |
| 5 | MGQKEQIHTLQKNSERMSKQLTRSSQAV |
| Mandarin | |
| fish TCE | |
| amino | |
| acid | |
| 6 | ATGGGCCAGAAAGAGCAGATCCACACACTGCAGAAAAACAGCGAGCGGATGAGCAA |
| TCE | GCAGCTGACCAGATCTTCTCAGGCCGTG |
| nucleic | |
| acid | |
| 7 | SHHHHHH |
| Ser-His | |
| tag amino | |
| acid | |
| 8 | AGCCATCACCATCACCACCAT |
| Ser-His | |
| tag | |
| nucleic | |
| acid | |
| 9 | MGQKEQIHTLQKNSERMSKQLTRSSQAVQNLQNGGGSRSSATLPGRRRRRWLRRRR |
| GAd20 | QPISVAPAGPPRRPNQKPNPPGGARCVIMRPTWPGTSAFTKRSFAVTERIIDYWAQ |
| Transgene | KEKGSSSFLRPSCDYWAQKEKISIPRTHLCLVLGVLSGHSGSRLYEAGMTLGGKIL |
| amino | FFLFLLLPLSPFSLIFTEISCCTLSSEENEYLPRPEWQLQVPFRELKNVSVLEGLR |
| acid with | QGRLGGPCSCHCPRPSQARLTPVDVAGPFLCLGDPGLFPPVKSSITGGKSTCSAPG |
| TCE and | PQSLPSTPFSTYPQWVILITELGMECTLGQVGAPSPRREEDGWRGGHSRFKADVPA |
| Ser-His | PQGPCWGGQPGSAPSSAPPEQSLLDWKFEMSYTVGGPPPHVHARPRHWKTDRDGHS |
| tag | YTSKVNCLLLQDGFHGCVSITGAAGRRNLSIFLFLMLCKLEFHACKIQNKNCPDFK |
| | KFDGPCGERGGGRTARALWARGDSVLTPALDPQTPVRAPSLTRAAAAVHYKLIQQP |
| | ISLFSITDRLHKTFSQLPSVHLCSITFQWGHPPIFCSTNDICVTANFCISVTFLKP |
| | CFLLHEASASQCHLFLQPQVGTPPPHTASARAPSGPPHPHESCPAGRRPARAAQTC |
| | ARRQHGLPGCEEAGTARVPSLHLHLHQAALGAGRGRGWGEACAQVPPSRGVLRFLD |
| | LKVRYLHSQWQHYHRSGEAAGTPLWRPTRNVPFRELKNQRTAQGAPGIHHAASPVA |
| | ANLCDPARHAQHTRIPCGAGQVRAGRGPEAGGGVLQPQRPAPEKPGCPCRRGQPRL |
| | HTVKMWRAVAMMVPDRQVHYDFGLGVPGDSTRRAVRRMNTFYEAGMTLGEKFRVGN |
| | CKHLKMTRPNSKMALNSEALSVVSECGASACDVSLIAMDSAFVQGKDWGVKKFIRR |
| | DFYAYKDFLWCFPFSLVFLQEIQICCHVSCLCCICCSTRICLGCLLELFLSRALRA |
| | LHVLWNGFQLHCQTEYNQKLQVNQFSESKSLYHREKQLIAMDSAICEERGAAGSLI |
| | SCETMPAILKLQKNCLLSLRTALTHNQDFSIYRLCCKRGSLCHASQARSPAFPKPV |
| | RPLPAPITRITPQLGGQSDSSQPLLTTGRPQGWQDQALRHTQQASPASCATITIPI |
| | HSAALGDHSGDPGPAWDTCPPLPLTTLIPRAPPPYGDSTARSWPSRCGPLGGNTTL |
| | QQLGEASQAPSGSLIPLRLPLLWEVRGQPDSFAALHSSLNELGEIARELHQFAFDL |
| | LIKSHFVQGKDWGLKKFIRRDFWGMELAASRRFSWDHHSAGGPPRVPSVRSGAAQV |
| | QPKDPLPLRTLAGCLARTAHLRPGAESLPQPQLHCTLWFQSSELSPTGAPWPSRRP |
| | TWRGTTVSPRTATSSARTCCGTKWPSSQEAALGLGSGLLRFSCGTAAIRKMHFSLK |
| | EHPPPPCPPEAFQRAAGEGGPGRGGARRGARVLQSPFCRAGAGEWLGHQSLRHVVG |
| | YGHLDTSGSSSSSSWPNSKMALNSLNSIDDAQLTRIAPPRSHCCFWEVNAPSHHHH |
| | HH |
| 10 | ATGGGCCAGAAAGAGCAGATCCACACACTGCAGAAAAACAGCGAGCGGATGAGCAA |
| GAd20 | GCAGCTGACCAGATCTTCTCAGGCCGTGCAGAACCTGCAGAACGGCGGAGGCTCTA |
| Transgene | GAAGCTCTGCTACACTTCCTGGCAGGCGGCGGAGAAGATGGCTGAGAAGAAGGCGG |
| nucleic | CAGCCTATCTCTGTGGCTCCTGCTGGACCTCCTAGACGGCCCAACCAGAAGCCTAA |
| acid with | TCCTCCTGGCGGAGCCAGATGCGTGATCATGAGGCCTACATGGCCTGGCACCAGCG |
| TCE and | CCTTCACCAAGAGAAGCTTTGCCGTGACCGAGCGGATCATCGACTATTGGGCTCAA |
| Ser-His | AAAGAGAAGGGCAGCAGCAGCTTCCTGCGGCCTAGCTGTGATTATTGGGCCCAGAA |
| tag | AGAAAAGATCAGCATCCCCAGAACACACCTGTGCCTGGTGCTGGGAGTGCTGTCTG |
| | GACACTCTGGCAGCAGACTGTATGAGGCCGGCATGACACTCGGCGGCAAGATCCTG |
| | TTCTTCCTGTTCCTGCTGCTCCCTCTGAGCCCCTTCAGCCTGATCTTCACCGAGAT |
| | CAGCTGCTGCACCCTGAGCAGCGAGGAAAACGAGTACCTGCCTAGACCTGAGTGGC |
| | AGCTGCAGGTCCCCTTCAGAGAGCTGAAGAACGTTTCCGTGCTGGAAGGCCTGAGA |
| | CAGGGCAGACTTGGCGGCCCTTGTAGCTGTCACTGCCCCAGACCTAGTCAGGCCAG |
| | ACTGACACCTGTGGATGTGGCCGGACCTTTCCTGTGTCTGGGAGATCCTGGCCTGT |
| | TTCCACCTGTGAAGTCCAGCATCACAGGCGGCAAGTCCACATGTTCTGCCCCTGGA |
| | CCTCAGAGCCTGCCTAGCACACCCTTCAGCACATACCCTCAGTGGGTCATCCTGAT |
| | CACCGAACTCGGCATGGAATGCACCCTGGGACAAGTGGGAGCCCCATCTCCTAGAA |
| | GAGAAGAGGATGGCTGGCGCGGAGGCCACTCTAGATTCAAAGCTGATGTGCCCGCT |
| | CCTCAGGGCCCTTGTTGGGGAGGACAACCTGGATCTGCCCCATCTTCTGCCCCACC |
| | TGAACAGTCCCTGCTGGATTGGAAGTTCGAGATGAGCTACACCGTCGGCGGACCTC |
| | CACCTCATGTTCATGCCAGACCTCGGCACTGGAAAACCGACAGAGATGGCCACAGC |
| | TACACCAGCAAAGTGAACTGCCTCCTGCTGCAGGATGGCTTCCACGGCTGTGTGTC |
| | TATTACTGGCGCCGCTGGCAGACGGAACCTGAGCATCTTTCTGTTTCTGATGCTGT |
| | GCAAGCTCGAGTTCCACGCCTGCAAGATCCAGAACAAGAACTGCCCCGACTTCAAG |
| | AAGTTCGACGGCCCTTGCGGAGAAAGAGGCGGAGGCAGAACAGCTAGAGCCCTTTG |
| | GGCTAGAGGCGACAGCGTTCTGACACCAGCTCTGGACCCTCAGACACCTGTTAGGG |
| | CCCCTAGCCTGACAAGAGCTGCCGCCGCTGTGCACTACAAGCTGATCCAGCAGCCA |
| | ATCAGCCTGTTCAGCATCACCGACCGGCTGCACAAGACATTCAGCCAGCTGCCAAG |
| | CGTGCACCTGTGCTCCATCACCTTCCAGTGGGGACACCCTCCTATCTTTTGCTCCA |
| | CCAACGACATCTGCGTGACCGCCAACTTCTGTATCAGCGTGACCTTCCTGAAGCCT |
| | TGCTTTCTGCTGCACGAGGCCAGCGCCTCTCAGTGCCACTTGTTTCTGCAGCCCCA |
| | AGTGGGCACACCTCCTCCACATACAGCCTCTGCTAGAGCACCTAGCGGCCCTCCAC |
| | ATCCTCACGAATCTTGTCCTGCCGGAAGAAGGCCTGCCAGAGCCGCTCAAACATGT |
| | GCCAGACGACAGCACGGACTGCCTGGATGTGAAGAGGCTGGAACAGCCAGAGTGCC |
| | TAGCCTGCACCTCCATCTGCATCAGGCTGCTCTTGGAGCCGGAAGAGGTAGAGGAT |
| | GGGGCGAAGCTTGTGCTCAGGTGCCACCTTCTAGAGGCGTGCTGAGATTCCTGGAC |
| | CTGAAAGTGCGCTACCTGCACAGCCAGTGGCAGCACTATCACAGATCTGGCGAAGC |
| | CGCCGGAACACCCCTTTGGAGGCCAACAAGAAACGTGCCCTTCCGGGAACTGAAGA |
| | ACCAGAGAACAGCTCAGGGCGCTCCTGGAATCCACCATGCTGCTTCTCCAGTGGCC |
| | GCCAACCTGTGTGATCCTGCCAGACATGCCCAGCACACCAGGATTCCTTGTGGCGC |
| | TGGACAAGTGCGCGCTGGAAGAGGACCTGAAGCAGGCGGAGGTGTTCTGCAACCTC |
| | AAAGACCCGCTCCTGAGAAGCCTGGCTGCCCTTGCAGAAGAGGACAGCCTAGACTG |
| | CACACCGTGAAAATGTGGCGAGCCGTGGCCATGATGGTGCCCGATAGACAGGTCCA |
| | CTACGACTTTGGACTGGGCGTGCCAGGCGATAGCACTCGGAGAGCCGTCAGACGGA |
| | TGAACACCTTTTACGAAGCCGGGATGACCCTGGGCGAGAAGTTCAGAGTGGGCAAC |
| | TGCAAGCACCTGAAGATGACCCGGCCTAACAGCAAGATGGCCCTGAATAGCGAGGC |
| | CCTGTCTGTGGTGTCTGAATGTGGCGCCTCTGCCTGTGACGTGTCCCTGATCGCTA |
| | TGGACTCCGCCTTTGTGCAGGGCAAAGACTGGGGCGTGAAGAAGTTCATCCGGCGG |
| | GACTTCTACGCCTACAAGGACTTCCTGTGGTGCTTCCCCTTCTCTCTGGTGTTCCT |
| | GCAAGAGATCCAGATCTGCTGTCATGTGTCCTGCCTGTGCTGCATCTGCTGTAGCA |
| | CCAGAATCTGCCTGGGCTGTCTGCTGGAACTGTTCCTGAGCAGAGCCCTGAGAGCA |
| | CTGCACGTGCTGTGGAACGGATTCCAGCTGCACTGCCAGACCGAGTACAACCAGAA |
| | ACTGCAAGTGAACCAGTTCAGCGAGAGCAAGAGCCTGTACCACCGGGAAAAGCAGC |
| | TCATTGCCATGGACAGCGCCATCTGCGAAGAGAGAGGCGCCGCAGGATCTCTGATC |
| | TCCTGCGAAACAATGCCCGCCATCCTGAAGCTGCAGAAGAATTGCCTCCTAAGCCT |
| | GCGAACCGCTCTGACACACAACCAGGACTTCAGCATCTACAGACTGTGTTGCAAGC |
| | GGGGCTCCCTGTGCCATGCAAGCCAAGCTAGAAGCCCCGCCTTTCCTAAACCTGTG |
| | CGACCTCTGCCAGCTCCAATCACCAGAATTACCCCTCAGCTCGGCGGCCAGAGCGA |
| | TTCATCTCAACCTCTGCTGACCACCGGCAGACCTCAAGGCTGGCAAGACCAAGCTC |
| | TGAGACACACCCAGCAGGCTAGCCCTGCCTCTTGTGCCACCATCACAATCCCCATC |
| | CACTCTGCCGCTCTGGGCGATCATTCTGGCGATCCTGGACCAGCCTGGGACACATG |
| | TCCTCCACTGCCACTCACAACACTGATCCCTAGGGCTCCTCCACCTTACGGCGATT |
| | CTACCGCTAGAAGCTGGCCCAGCAGATGTGGACCACTCGGAGGCAACACAACCCTC |
| | CAGCAACTGGGAGAAGCCTCTCAGGCTCCTAGCGGCTCTCTGATCCCTCTCAGACT |
| | GCCTCTCCTGTGGGAAGTTCGGGGCCAGCCTGATTCTTTTGCCGCACTGCACAGCT |
| | CCCTGAACGAGCTGGGAGAGATCGCTAGAGAGCTGCACCAGTTCGCCTTCGACCTG |
| | CTGATCAAGAGCCACTTCGTGCAAGGCAAGGATTGGGGCCTCAAAAAGTTTATCCG |
| | CAGAGACTTCTGGGGCATGGAACTGGCCGCCAGCAGAAGATTCAGCTGGGATCATC |
| | ATAGCGCAGGCGGCCCACCTAGAGTGCCATCTGTTAGAAGCGGAGCTGCCCAGGTG |
| | CAGCCTAAAGATCCTCTGCCACTGAGAACACTGGCCGGCTGCCTTGCTAGAACAGC |
| | CCATCTTAGACCTGGCGCCGAGTCTCTGCCTCAGCCACAACTGCACTGTACCCTGT |
| | GGTTCCAGTCCAGCGAGCTGTCTCCTACTGGTGCCCCTTGGCCATCTAGACGCCCT |
| | ACTTGGAGAGGCACCACCGTGTCACCAAGAACCGCCACAAGCAGCGCCAGAACCTG |
| | TTGTGGCACAAAGTGGCCCTCCAGCCAAGAAGCCGCTCTCGGACTTGGAAGCGGAC |
| | TGCTGAGGTTCTCTTGTGGAACCGCCGCCATTCGGAAGATGCACTTTAGCCTGAAA |
| | GAACACCCTCCACCACCTTGTCCTCCAGAGGCTTTCCAAAGAGCTGCTGGCGAAGG |
| | CGGACCTGGTAGAGGTGGTGCTAGAAGAGGTGCTAGGGTGCTGCAGAGCCCATTCT |
| | GTAGAGCAGGCGCAGGCGAATGGCTGGGCCATCAGAGTCTGAGACATGTCGTCGGC |
| | TACGGCCACCTGGATACAAGCGGAAGCAGCTCTAGCTCCAGCTGGCCTAACTCAAA |
| | AATGGCTCTGAACAGCCTGAACTCCATCGACGACGCCCAGCTGACAAGAATCGCCC |
| | CTCCTAGATCTCACTGCTGCTTTTGGGAAGTGAACGCCCCAAGCCATCACCATCAC |
| | CACCAT |
| 11 | MGQKEQIHTLQKNSERMSKQLTRSSQAVQNLQNGGGSRSSATLPGRRRRRWLRRRR |
| Transgene | QPISVAPAGPPRRPNQKPNPPGGARCVIMRPTWPGTSAFTGMECTLGQVGAPSPRR |
| amino | EEDGWRGGHSRFKADVPAPQGPCWGGQPGSAPSSAPPEQSLLDDYWAQKEKISIPR |
| acid with | THLCWKFEMSYTVGGPPPHVHARPRHWKTDRDYWAQKEKGSSSFLRPSCVPFRELK |
| TCE and | NVSVLEGLRQGRLGGPCSCHCPRPSQARLTPVDVAGPFLCLGDPGLFPPVKSSITE |
| Ser-His | ISCCTLSSEENEYLPRPEWQLQYEAGMTLGGKILFFLFLLLPLSPFSLIFLVLGVL |
| tag | SGHSGSRLKRSFAVTERIITGGKSTCSAPGPQSLPSTPFSTYPQWVILITELDGHS |
| MVA | YTSKVNCLLLQDGFHGCVSITGAAGRRNLSIFLFLMLCKLEFHACQWQHYHRSGEA |
| | AGTPLWRPTRNVAMMVPDRQVHYDFGLKIQNKNCPDVLRFLDLKVRYLHSVPFREL |
| | KNQRTAQGAPGIHHAASPVAANLCDPARHAQHTRIPCGAGQVRAGRGPEAGGGVLQ |
| | PQRPAPEKPGCPCRRGQPRLHTVKMWRACHLFLQPQVGTPPPHTASARAPSGPPHP |
| | HESCPAGRRPARAAQTCARRQHGLPGCEEAGTARVPSLHLHLHQAALGAGRGRGWG |
| | EACAQVPPSRGHYKLIQQPISLFSITDRLHKTFSQLPSVHLCSITFQWGHPPIFCS |
| | TNDICVTANFCISVTFLKPCFLLHEASASQFKKFDGPCGERGGGRTARALWARGDS |
| | VLTPALDPQTPVRAPSLTRAAAAVGVPGDSTRRAVRRMNTFCGASACDVSLIAMDS |
| | ACEERGAAGSLISCESLYHREKQLIAMDSAIFVQGKDWGVKKFIRRDFTMPAILKL |
| | QKNCLLSLNSKMALNSEALSVVSEYEAGMTLGEKFRVGNCKHLKMTRPTEYNQKLQ |
| | VNQFSESKRTALTHNQDFSIYRLCCKRGSLCHASQARSPAFPKPVRPLPAPITRIT |
| | PQLGGQSDSSQPLLTTGRPQGWQDQALRHTQQASPASCATITIPIHSAALGDHSGD |
| | PGPAWDTCPPLPLTTLIPRAPPPYGDSTARSWPSRCGPLGYAYKDFLWCFPFSLVF |
| | LQEIQICCHVSCLCCICCSTRICLGCLLELFLSRALRALHVLWNGFQLHCQGNTTL |
| | QQLGEASQAPSGSLIPLRLPLLWEVRGNSKMALNSLNSIDDAQLTRIAPPRSHCCF |
| | WEVNAPFVQGKDWGLKKFIRRDFEAFQRAAGEGGPGRGGARRGARVLQSPFCRAGA |
| | GEWLGHQSLRWGMELAASRRFSWDHHSAGGPPRVPSVRSGAAQVQPKDPLPLRTLA |
| | GCLARTAHLRPGAESLPQPQLHCTIARELHQFAFDLLIKSHKMHFSLKEHPPPPCP |
| | PHVVGYGHLDTSGSSSSSSWPQPDSFAALHSSLNELGELWFQSSELSPTGAPWPSR |
| | RPTWRGTTVSPRTATSSARTCCGTKWPSSQEAALGLGSGLLRFSCGTAAIRSHHHH |
| | HH |
| 12 | ATGGGCCAGAAAGAGCAGATCCACACACTGCAGAAAAACAGCGAGCGGATGAGCAA |
| MVA | GCAGCTGACCAGATCTTCTCAGGCCGTGCAGAACCTGCAGAACGGCGGAGGCTCTA |
| Transgene | GAAGCTCTGCTACACTTCCTGGCAGGCGGCGGAGAAGATGGCTGAGAAGAAGGCGG |
| nucleic | CAGCCTATCTCTGTGGCTCCTGCTGGACCTCCTAGACGGCCCAACCAGAAGCCTAA |
| acid with | TCCTCCTGGCGGAGCCAGATGCGTGATCATGAGGCCTACATGGCCTGGCACCAGCG |
| TCE and | CCTTTACCGGCATGGAATGTACACTGGGCCAAGTGGGAGCCCCATCTCCTAGAAGA |
| Ser-His | GAAGAGGATGGCTGGCGCGGAGGCCACTCTAGATTCAAAGCTGATGTGCCCGCTCC |
| tag | TCAGGGCCCTTGTTGGGGAGGACAACCTGGATCTGCCCCATCTTCTGCCCCACCTG |
| | AACAGAGCCTGCTGGATGACTATTGGGCTCAAAAAGAGAAGATCAGCATCCCCAGA |
| | ACACACCTGTGCTGGAAGTTCGAGATGAGCTACACCGTTGGCGGCCCTCCACCACA |
| | TGTTCACGCCAGACCTAGACACTGGAAAACCGACAGAGATTATTGGGCCCAGAAAG |
| | AAAAGGGCAGCAGCAGCTTCCTGCGGCCTAGCTGTGTGCCCTTCCGGGAACTGAAG |
| | AACGTGTCCGTTCTGGAAGGCCTGAGGCAGGGCAGACTTGGCGGACCTTGTAGCTG |
| | CCACTGTCCTAGACCAAGCCAGGCCAGACTGACCCCTGTGGATGTGGCTGGCCCAT |
| | TTCTGTGTCTGGGCGACCCTGGACTGTTCCCTCCAGTGAAGTCTAGCATCACCGAG |
| | ATCAGCTGCTGCACCCTGAGCAGCGAGGAAAACGAGTACCTGCCTAGACCTGAATG |
| | GCAGCTGCAGTACGAGGCCGGCATGACACTCGGAGGCAAGATCCTGTTCTTCCTGT |
| | TCCTGCTGCTCCCTCTGAGCCCCTTCAGCCTGATCTTTCTGGTGCTGGGCGTGCTG |
| | TCTGGCCACTCTGGAAGCAGACTGAAGAGAAGCTTCGCCGTGACCGAGCGGATCAT |
| | CACAGGCGGCAAGAGCACATGTTCTGCCCCTGGACCTCAGTCTCTGCCCAGCACAC |
| | CCTTCAGCACATACCCTCAGTGGGTCATCCTGATCACCGAGCTGGATGGCCACAGC |
| | TACACCAGCAAAGTGAACTGCCTCCTGCTGCAGGATGGCTTCCACGGCTGTGTGTC |
| | TATTACTGGCGCCGCTGGCAGACGGAACCTGAGCATCTTTCTGTTTCTGATGCTGT |
| | GCAAGCTCGAGTTCCACGCCTGCCAATGGCAGCACTACCACAGATCTGGCGAAGCC |
| | GCTGGAACCCCACTTTGGAGGCCTACCAGAAACGTGGCCATGATGGTGCCCGACAG |
| | ACAGGTGCACTACGACTTCGGCCTGAAGATCCAGAACAAGAACTGCCCCGACGTGC |
| | TGCGGTTCCTGGATCTCAAAGTGCGCTACCTGCACAGCGTGCCCTTCAGAGAGCTG |
| | AAAAACCAGAGAACAGCCCAGGGCGCTCCTGGAATCCATCATGCTGCTTCTCCAGT |
| | GGCCGCCAATCTGTGCGATCCTGCCAGACATGCCCAGCATACCAGGATTCCTTGTG |
| | GCGCTGGACAAGTGCGCGCTGGAAGAGGACCTGAAGCTGGTGGCGGAGTTCTGCAG |
| | CCTCAAAGACCTGCTCCTGAGAAGCCTGGCTGCCCCTGTAGAAGAGGACAGCCTAG |
| | ACTGCACACCGTGAAGATGTGGCGGGCCTGCCACTTGTTTCTCCAGCCACAAGTGG |
| | GCACCCCTCCACCTCATACAGCCTCTGCTAGAGCACCTAGCGGCCCACCTCATCCT |
| | CACGAATCTTGTCCTGCCGGAAGAAGGCCTGCCAGAGCCGCTCAAACATGTGCCAG |
| | ACGACAGCACGGACTGCCCGGATGTGAAGAAGCCGGAACAGCCAGAGTGCCTAGCC |
| | TGCACCTTCATCTGCATCAGGCCGCTCTTGGAGCCGGAAGAGGTAGAGGATGGGGA |
| | GAAGCTTGTGCCCAGGTGCCACCTTCTAGAGGCCACTACAAGCTGATCCAGCAGCC |
| | AATCAGCCTGTTCTCCATCACCGACCGGCTGCACAAGACATTCAGCCAGCTGCCTT |
| | CCGTGCATCTGTGCAGCATCACCTTCCAGTGGGGACACCCTCCTATCTTTTGCTCC |
| | ACCAACGACATCTGCGTGACCGCCAACTTCTGTATCAGCGTGACCTTCCTGAAGCC |
| | TTGCTTTCTGCTGCACGAGGCCTCCGCCAGCCAGTTTAAGAAGTTTGACGGCCCCT |
| | GCGGCGAGAGAGGCGGAGGAAGAACTGCAAGAGCCCTTTGGGCCAGAGGCGACTCT |
| | GTTCTGACACCAGCTCTGGACCCTCAGACACCTGTTAGGGCCCCTAGCCTGACAAG |
| | AGCTGCCGCTGCTGTTGGAGTGCCTGGCGATTCTACTAGAAGGGCCGTGCGGCGGA |
| | TGAACACCTTTTGTGGCGCATCTGCCTGCGACGTGTCCCTGATCGCTATGGATAGC |
| | GCCTGCGAGGAAAGAGGCGCAGCCGGATCTCTGATCTCTTGCGAGAGCCTGTACCA |
| | CCGGGAAAAGCAGCTCATTGCCATGGACAGCGCCATCTTCGTGCAGGGCAAAGACT |
| | GGGGCGTGAAGAAGTTCATCCGGCGGGACTTTACCATGCCTGCCATTCTGAAGCTG |
| | CAGAAGAATTGTCTTCTAAGCCTGAACAGCAAGATGGCCCTGAATAGCGAGGCCCT |
| | GTCTGTGGTGTCCGAGTATGAGGCTGGAATGACCCTGGGCGAGAAGTTCAGAGTGG |
| | GCAACTGCAAGCACCTGAAGATGACCCGGCCTACCGAGTACAACCAGAAACTGCAA |
| | GTGAACCAGTTCAGCGAGAGCAAGCGGACCGCTCTGACCCACAACCAGGACTTCAG |
| | CATCTACCGGCTGTGCTGCAAGAGGGGCTCTCTGTGTCATGCTAGCCAGGCTAGAA |
| | GCCCCGCCTTTCCTAAGCCTGTCAGACCTCTGCCTGCTCCTATCACCAGAATCACC |
| | CCTCAGCTCGGCGGCCAGTCTGATTCATCTCAGCCACTGCTGACCACCGGCAGACC |
| | TCAAGGATGGCAAGACCAGGCTCTGAGACACACACAGCAGGCTAGCCCAGCCTCTT |
| | GCGCCACCATCACAATACCAATACATTCTGCCGCTCTGGGCGATCACAGCGGAGAT |
| | CCTGGACCTGCCTGGGATACTTGTCCTCCTCTGCCCCTAACTACACTGATCCCTAG |
| | GGCTCCTCCACCTTACGGCGATAGCACAGCCAGATCCTGGCCTAGCAGATGTGGCC |
| | CTCTGGGCTACGCCTACAAGGACTTCCTGTGGTGCTTCCCCTTCTCTCTGGTGTTC |
| | CTGCAAGAAATCCAGATCTGCTGTCACGTGTCCTGCCTGTGCTGTATCTGCTGTAG |
| | CACCCGGATCTGTCTGGGCTGTCTGCTGGAACTGTTCCTGAGCAGAGCCCTGAGAG |
| | CACTGCACGTGCTGTGGAACGGATTCCAGCTGCACTGCCAGGGCAACACCACACTG |
| | CAACAGCTGGGAGAAGCCTCTCAGGCCCCAAGCGGTTCTCTGATCCCTCTCAGACT |
| | GCCCCTCCTGTGGGAAGTGCGGGGCAATTCTAAGATGGCTCTCAACAGCCTGAACT |
| | CCATCGACGACGCCCAGCTGACAAGAATCGCCCCTCCAAGAAGCCACTGTTGCTTT |
| | TGGGAAGTGAACGCCCCTTTTGTGCAGGGTAAAGATTGGGGCCTCAAAAAGTTTAT |
| | CAGACGGGACTTCGAGGCTTTCCAGAGAGCAGCTGGCGAAGGCGGACCTGGCAGAG |
| | GTGGTGCTAGAAGAGGTGCTAGAGTGCTGCAGAGCCCATTCTGTAGAGCTGGCGCT |
| | GGCGAATGGCTGGGCCACCAATCTCTTAGATGGGGAATGGAACTGGCCGCTAGCAG |
| | GCGGTTTAGCTGGGATCATCATTCTGCCGGCGGACCTCCAAGAGTGCCAAGCGTTA |
| | GAAGCGGAGCAGCCCAGGTCCAGCCTAAAGATCCACTGCCACTGAGAACACTGGCC |
| | GGCTGCCTTGCCAGAACAGCTCATCTTAGACCTGGCGCCGAAAGCCTGCCTCAACC |
| | TCAGCTGCATTGCACAATCGCCAGAGAACTGCACCAGTTCGCCTTCGACCTGCTGA |
| | TCAAGAGCCACAAGATGCACTTCTCACTGAAAGAGCACCCGCCACCGCCGTGCCCA |
| | CCGCACGTTGTCGGCTATGGCCACCTGGATACAAGCGGCTCCTCTAGCAGTAGCTC |
| | CTGGCCTCAGCCTGACAGCTTTGCTGCCCTGCATAGCTCCCTGAATGAGCTGGGCG |
| | AACTGTGGTTCCAGTCCAGCGAACTGTCTCCTACTGGCGCTCCATGGCCAAGCAGA |
| | AGGCCTACTTGGAGAGGCACCACCGTGTCTCCAAGAACCGCTACAAGCAGCGCCAG |
| | AACCTGTTGCGGCACAAAATGGCCCTCCAGCCAAGAAGCTGCCCTCGGACTTGGAA |
| | GCGGACTGCTGAGATTCAGCTGTGGCACAGCCGCCATCAGAAGCCATCACCATCAC |
| | CACCAT |
| 13 | QNLQNGGGSRSSATLPGRRRRRWLRRRRQPISVAPAGPPRRPNQKPNPPGGARCVI |
| FR1 amino | MRPTWPGTSAFT |
| acid | |
| 14 | CAGAACCTGCAGAACGGCGGAGGCTCTAGAAGCTCTGCTACACTTCCTGGCAGGCG |
| FR1 | GCGGAGAAGATGGCTGAGAAGAAGGCGGCAGCCTATCTCTGTGGCTCCTGCTGGAC |
| nucleic | CTCCTAGACGGCCCAACCAGAAGCCTAATCCTCCTGGCGGAGCCAGATGCGTGATC |
| acid | ATGAGGCCTACATGGCCTGGCACCAGCGCCTTCACC |
| 15 | KRSFAVTERII |
| AS13 | |
| amino | |
| acid | |
| 16 | AAGAGAAGCTTTGCCGTGACCGAGCGGATCATC |
| AS13 | |
| nucleic | |
| acid | |
| 17 | DYWAQKEKGSSSFLRPSC |
| AS7 amino | |
| acid | |
| 18 | GACTATTGGGCTCAAAAAGAGAAGGGCAGCAGCAGCTTCCTGCGGCCTAGCTGT |
| AS7 | |
| nucleic | |
| acid | |
| 19 | DYWAQKEKISIPRTHLC |
| AS6 amino | |
| acid | |
| 20 | GATTATTGGGCCCAGAAAGAAAAGATCAGCATCCCCAGAACACACCTGTGC |
| AS6 | |
| nucleic | |
| acid | |
| 21 | LVLGVLSGHSGSRL |
| AS8 amino | |
| acid | |
| 22 | CTGGTGCTGGGAGTGCTGTCTGGACACTCTGGCAGCAGACTG |
| AS8 | |
| nucleic | |
| acid | |
| 23 | YEAGMTLGGKILFFLFLLLPLSPFSLIF |
| AS62 | |
| amino | |
| acid | |
| 24 | TATGAGGCCGGCATGACACTCGGCGGCAAGATCCTGTTCTTCCTGTTCCTGCTGCT |
| AS62 | CCCTCTGAGCCCCTTCAGCCTGATCTTC |
| nucleic | |
| acid | |
| 25 | TEISCCTLSSEENEYLPRPEWQLQ |
| FUS3 | |
| amino | |
| acid | |
| 26 | ACCGAGATCAGCTGCTGCACCCTGAGCAGCGAGGAAAACGAGTACCTGCCTAGACC |
| FUS3 | TGAGTGGCAGCTGCAG |
| nucleic | |
| acid | |
| 27 | VPFRELKNVSVLEGLRQGRLGGPCSCHCPRPSQARLTPVDVAGPFLCLGDPGLFPP |
| AS43 | VKSSI |
| amino | |
| acid | |
| 28 | GTCCCCTTCAGAGAGCTGAAGAACGTTTCCGTGCTGGAAGGCCTGAGACAGGGCAG |
| AS43 | ACTTGGCGGCCCTTGTAGCTGTCACTGCCCCAGACCTAGTCAGGCCAGACTGACAC |
| nucleic | CTGTGGATGTGGCCGGACCTTTCCTGTGTCTGGGAGATCCTGGCCTGTTTCCACCT |
| acid | GTGAAGTCCAGCATC |
| 29 | TGGKSTCSAPGPQSLPSTPFSTYPQWVILITEL |
| AS57 | |
| amino | |
| acid | |
| 30 | ACAGGCGGCAAGTCCACATGTTCTGCCCCTGGACCTCAGAGCCTGCCTAGCACACC |
| AS57 | CTTCAGCACATACCCTCAGTGGGTCATCCTGATCACCGAACTC |
| nucleic | |
| acid | |
| 31 | GMECTLGQVGAPSPRREEDGWRGGHSRFKADVPAPQGPCWGGQPGSAPSSAPPEQS |
| AS51 | LLD |
| amino | |
| acid | |
| 32 | GGCATGGAATGCACCCTGGGACAAGTGGGAGCCCCATCTCCTAGAAGAGAAGAGGA |
| AS51 | TGGCTGGCGCGGAGGCCACTCTAGATTCAAAGCTGATGTGCCCGCTCCTCAGGGCC |
| nucleic | CTTGTTGGGGAGGACAACCTGGATCTGCCCCATCTTCTGCCCCACCTGAACAGTCC |
| acid | CTGCTGGAT |
| 33 | WKFEMSYTVGGPPPHVHARPRHWKTDR |
| AS18 | |
| amino | |
| acid | |
| 34 | TGGAAGTTCGAGATGAGCTACACCGTCGGCGGACCTCCACCTCATGTTCATGCCAG |
| AS18 | ACCTCGGCACTGGAAAACCGACAGA |
| nucleic | |
| acid | |
| 35 | DGHSYTSKVNCLLLQDGFHGCVSITGAAGRRNLSIFLFLMLCKLEFHAC |
| AS64 | |
| amino | |
| acid | |
| 36 | GATGGCCACAGCTACACCAGCAAAGTGAACTGCCTCCTGCTGCAGGATGGCTTCCA |
| AS64 | CGGCTGTGTGTCTATTACTGGCGCCGCTGGCAGACGGAACCTGAGCATCTTTCTGT |
| nucleic | TTCTGATGCTGTGCAAGCTCGAGTTCCACGCCTGC |
| acid | |
| 37 | KIQNKNCPD |
| AS23 | |
| amino | |
| acid | |
| 38 | AAGATCCAGAACAAGAACTGCCCCGAC |
| AS23 | |
| nucleic | |
| acid | |
| 39 | FKKFDGPCGERGGGRTARALWARGDSVLTPALDPQTPVRAPSLTRAAAAV |
| AS47 | |
| amino | |
| acid | |
| 40 | TTCAAGAAGTTCGACGGCCCTTGCGGAGAAAGAGGCGGAGGCAGAACAGCTAGAGC |
| AS47 | CCTTTGGGCTAGAGGCGACAGCGTTCTGACACCAGCTCTGGACCCTCAGACACCTG |
| nucleic | TTAGGGCCCCTAGCCTGACAAGAGCTGCCGCCGCTGTG |
| acid | |
| 41 | HYKLIQQPISLFSITDRLHKTFSQLPSVHLCSITFQWGHPPIFCSTNDICVTANFC |
| CAS1 | ISVTFLKPCFLLHEASASQ |
| amino | |
| acid | |
| 42 | CACTACAAGCTGATCCAGCAGCCAATCAGCCTGTTCAGCATCACCGACCGGCTGCA |
| CAS1 | CAAGACATTCAGCCAGCTGCCAAGCGTGCACCTGTGCTCCATCACCTTCCAGTGGG |
| nucleic | GACACCCTCCTATCTTTTGCTCCACCAACGACATCTGCGTGACCGCCAACTTCTGT |
| acid | ATCAGCGTGACCTTCCTGAAGCCTTGCTTTCTGCTGCACGAGGCCAGCGCCTCTCA |
| | G |
| 43 | CHLFLQPQVGTPPPHTASARAPSGPPHPHESCPAGRRPARAAQTCARRQHGLPGCE |
| AS37 | EAGTARVPSLHLHLHQAALGAGRGRGWGEACAQVPPSRG |
| amino | |
| acid | |
| 44 | TGCCACTTGTTTCTGCAGCCCCAAGTGGGCACACCTCCTCCACATACAGCCTCTGC |
| AS37 | TAGAGCACCTAGCGGCCCTCCACATCCTCACGAATCTTGTCCTGCCGGAAGAAGGC |
| nucleic | CTGCCAGAGCCGCTCAAACATGTGCCAGACGACAGCACGGACTGCCTGGATGTGAA |
| acid | GAGGCTGGAACAGCCAGAGTGCCTAGCCTGCACCTCCATCTGCATCAGGCTGCTCT |
| | TGGAGCCGGAAGAGGTAGAGGATGGGGCGAAGCTTGTGCTCAGGTGCCACCTTCTA |
| | GAGGC |
| 45 | VLRFLDLKVRYLHS |
| AS15 | |
| amino | |
| acid | |
| 46 | GTGCTGAGATTCCTGGACCTGAAAGTGCGCTACCTGCACAGC |
| AS15 | |
| nucleic | |
| acid | |
| 47 | QWQHYHRSGEAAGTPLWRPTRN |
| AS19 | |
| amino | |
| acid | |
| 48 | CAGTGGCAGCACTATCACAGATCTGGCGAAGCCGCCGGAACACCCCTTTGGAGGCC |
| AS19 | AACAAGAAAC |
| nucleic | |
| acid | |
| 49 | VPFRELKNQRTAQGAPGIHHAASPVAANLCDPARHAQHTRIPCGAGQVRAGRGPEA |
| AS11 | GGGVLQPQRPAPEKPGCPCRRGQPRLHTVKMWRA |
| amino | |
| acid | |
| 50 | GTGCCCTTCCGGGAACTGAAGAACCAGAGAACAGCTCAGGGCGCTCCTGGAATCCA |
| AS11 | CCATGCTGCTTCTCCAGTGGCCGCCAACCTGTGTGATCCTGCCAGACATGCCCAGC |
| nucleic | ACACCAGGATTCCTTGTGGCGCTGGACAAGTGCGCGCTGGAAGAGGACCTGAAGCA |
| acid | GGCGGAGGTGTTCTGCAACCTCAAAGACCCGCTCCTGAGAAGCCTGGCTGCCCTTG |
| | CAGAAGAGGACAGCCTAGACTGCACACCGTGAAAATGTGGCGAGCC |
| 51 | VAMMVPDRQVHYDFGL |
| AS3 amino | |
| acid | |
| 52 | GTGGCCATGATGGTGCCCGATAGACAGGTCCACTACGACTTTGGACTG |
| AS3 | |
| nucleic | |
| acid | |
| 53 | GVPGDSTRRAVRRMNTF |
| FUS27 | |
| amino | |
| acid | |
| 54 | GGCGTGCCAGGCGATAGCACTCGGAGAGCCGTCAGACGGATGAACACCTTT |
| FUS27 | |
| nucleic | |
| acid | |
| 55 | YEAGMTLGEKFRVGNCKHLKMTRP |
| AS61 | |
| amino | |
| acid | |
| 56 | TACGAAGCCGGGATGACCCTGGGCGAGAAGTTCAGAGTGGGCAACTGCAAGCACCT |
| AS61 | GAAGATGACCCGGCCT |
| nucleic | |
| acid | |
| 57 | NSKMALNSEALSVVSE |
| FUS5 | |
| amino | |
| acid | |
| 58 | AACAGCAAGATGGCCCTGAATAGCGAGGCCCTGTCTGTGGTGTCTGAA |
| FUS5 | |
| nucleic | |
| acid | |
| 59 | CGASACDVSLIAMDSA |
| FUS1 | |
| amino | |
| acid | |
| 60 | TGTGGCGCCTCTGCCTGTGACGTGTCCCTGATCGCTATGGACTCCGCC |
| FUS1 | |
| nucleic | |
| acid | |
| 61 | FVQGKDWGVKKFIRRDF |
| M12 amino | |
| acid | |
| 62 | TTTGTGCAGGGCAAAGACTGGGGCGTGAAGAAGTTCATCCGGCGGGACTTC |
| M12 | |
| nucleic | |
| acid | |
| 63 | YAYKDFLWCFPFSLVFLQEIQICCHVSCLCCICCSTRICLGCLLELFLSRALRALH |
| CAS3 | VLWNGFQLHCQ |
| amino | |
| acid | |
| 64 | TACGCCTACAAGGACTTCCTGTGGTGCTTCCCCTTCTCTCTGGTGTTCCTGCAAGA |
| CAS3 | GATCCAGATCTGCTGTCATGTGTCCTGCCTGTGCTGCATCTGCTGTAGCACCAGAA |
| nucleic | TCTGCCTGGGCTGTCTGCTGGAACTGTTCCTGAGCAGAGCCCTGAGAGCACTGCAC |
| acid | GTGCTGTGGAACGGATTCCAGCTGCACTGCCAG |
| 65 | TEYNQKLQVNQFSESK |
| FUS2 | |
| amino | |
| acid | |
| 66 | ACCGAGTACAACCAGAAACTGCAAGTGAACCAGTTCAGCGAGAGCAAG |
| FUS2 | |
| nucleic | |
| acid | |
| 67 | SLYHREKQLIAMDSAI |
| FUS29 | |
| amino | |
| acid | |
| 68 | AGCCTGTACCACCGGGAAAAGCAGCTCATTGCCATGGACAGCGCCATC |
| FUS29 | |
| nucleic | |
| acid | |
| 69 | CEERGAAGSLISCE |
| FUS6 | |
| amino | |
| acid | |
| 70 | TGCGAAGAGAGAGGCGCCGCAGGATCTCTGATCTCCTGCGAA |
| FUS6 | |
| nucleic | |
| acid | |
| 71 | TMPAILKLQKNCLLSL |
| CAS4 | |
| amino | |
| acid | |
| 72 | ACAATGCCCGCCATCCTGAAGCTGCAGAAGAATTGCCTCCTAAGCCTG |
| CAS4 | |
| nucleic | |
| acid | |
| 73 | RTALTHNQDFSIYRLCCKRGSLCHASQARSPAFPKPVRPLPAPITRITPQLGGQSD |
| CAS2 | SSQPLLTTGRPQGWQDQALRHTQQASPASCATITIPIHSAALGDHSGDPGPAWDTC |
| amino | PPLPLTTLIPRAPPPYGDSTARSWPSRCGPLG |
| acid | |
| 74 | CGAACCGCTCTGACACACAACCAGGACTTCAGCATCTACAGACTGTGTTGCAAGCG |
| CAS2 | GGGCTCCCTGTGCCATGCAAGCCAAGCTAGAAGCCCCGCCTTTCCTAAACCTGTGC |
| nucleic | GACCTCTGCCAGCTCCAATCACCAGAATTACCCCTCAGCTCGGCGGCCAGAGCGAT |
| acid | TCATCTCAACCTCTGCTGACCACCGGCAGACCTCAAGGCTGGCAAGACCAAGCTCT |
| | GAGACACACCCAGCAGGCTAGCCCTGCCTCTTGTGCCACCATCACAATCCCCATCC |
| | ACTCTGCCGCTCTGGGCGATCATTCTGGCGATCCTGGACCAGCCTGGGACACATGT |
| | CCTCCACTGCCACTCACAACACTGATCCCTAGGGCTCCTCCACCTTACGGCGATTC |
| | TACCGCTAGAAGCTGGCCCAGCAGATGTGGACCACTCGGA |
| 75 | GNTTLQQLGEASQAPSGSLIPLRLPLLWEVRG |
| AS16 | |
| amino | |
| acid | |
| 76 | GGCAACACAACCCTCCAGCAACTGGGAGAAGCCTCTCAGGCTCCTAGCGGCTCTCT |
| AS16 | GATCCCTCTCAGACTGCCTCTCCTGTGGGAAGTTCGGGGC |
| nucleic | |
| acid | |
| 77 | QPDSFAALHSSLNELGE |
| M86 amino | |
| acid | |
| 78 | CAGCCTGATTCTTTTGCCGCACTGCACAGCTCCCTGAACGAGCTGGGAGAG |
| M86 | |
| nucleic | |
| acid | |
| 79 | IARELHQFAFDLLIKSH |
| M84 amino | |
| acid | |
| 80 | ATCGCTAGAGAGCTGCACCAGTTCGCCTTCGACCTGCTGATCAAGAGCCAC |
| M84 | |
| nucleic | |
| acid | |
| 81 | FVQGKDWGLKKFIRRDF |
| M10 amino | |
| acid | |
| 82 | TTCGTGCAAGGCAAGGATTGGGGCCTCAAAAAGTTTATCCGCAGAGACTTC |
| M10 | |
| nucleic | |
| acid | |
| 83 | WGMELAASRRFSWDHHSAGGPPRVPSVRSGAAQVQPKDPLPLRTLAGCLARTAHLR |
| FUS8 | PGAESLPQPQLHCT |
| amino | |
| acid | |
| 84 | TGGGGCATGGAACTGGCCGCCAGCAGAAGATTCAGCTGGGATCATCATAGCGCAGG |
| FUS8 | CGGCCCACCTAGAGTGCCATCTGTTAGAAGCGGAGCTGCCCAGGTGCAGCCTAAAG |
| nucleic | ATCCTCTGCCACTGAGAACACTGGCCGGCTGCCTTGCTAGAACAGCCCATCTTAGA |
| acid | CCTGGCGCCGAGTCTCTGCCTCAGCCACAACTGCACTGTACC |
| 85 | LWFQSSELSPTGAPWPSRRPTWRGTTVSPRTATSSARTCCGTKWPSSQEAALGLGS |
| FUS7 | GLLRFSCGTAAIR |
| amino | |
| acid | |
| 86 | CTGTGGTTCCAGTCCAGCGAGCTGTCTCCTACTGGTGCCCCTTGGCCATCTAGACG |
| FUS7 | CCCTACTTGGAGAGGCACCACCGTGTCACCAAGAACCGCCACAAGCAGCGCCAGAA |
| nucleic | CCTGTTGTGGCACAAAGTGGCCCTCCAGCCAAGAAGCCGCTCTCGGACTTGGAAGC |
| acid | GGACTGCTGAGGTTCTCTTGTGGAACCGCCGCCATTCGG |
| 87 | KMHFSLKEHPPPPCPP |
| FUS19 | |
| amino | |
| acid | |
| 88 | AAGATGCACTTTAGCCTGAAAGAACACCCTCCACCACCTTGTCCTCCA |
| FUS19 | |
| nucleic | |
| acid | |
| 89 | EAFQRAAGEGGPGRGGARRGARVLQSPFCRAGAGEWLGHQSLR |
| AS41 | |
| amino | |
| acid | |
| 90 | GAGGCTTTCCAAAGAGCTGCTGGCGAAGGCGGACCTGGTAGAGGTGGTGCTAGAAG |
| AS41 | AGGTGCTAGGGTGCTGCAGAGCCCATTCTGTAGAGCAGGCGCAGGCGAATGGCTGG |
| nucleic | GCCATCAGAGTCTGAGA |
| acid | |
| 91 | HVVGYGHLDTSGSSSSSSWP |
| FUS15 | |
| amino | |
| acid | |
| 92 | CATGTCGTCGGCTACGGCCACCTGGATACAAGCGGAAGCAGCTCTAGCTCCAGCTG |
| FUS15 | GCCT |
| nucleic | |
| acid | |
| 93 | NSKMALNSLNSIDDAQLTRIAPPRSHCCFWEVNAP |
| FUS31 | |
| amino | |
| acid | |
| 94 | AACTCAAAAATGGCTCTGAACAGCCTGAACTCCATCGACGACGCCCAGCTGACAAG |
| FUS31 | AATCGCCCCTCCTAGATCTCACTGCTGCTTTTGGGAAGTGAACGCCCCA |
| nucleic | |
| acid | |

### Embodiments

The following list of embodiments is intended to complement, rather than displace or supersede, the previous descriptions.

Embodiment 1. A method of treating or preventing prostate cancer in a subject, the method comprising administering to the subject a treatment regimen comprising:

two or more vaccines comprising a great ape adenovirus serotype 20 (GAd20) virus that, in turn, comprises a nucleotide sequence encoding the amino acid sequence of SEQ ID NO: 1 and

one or more vaccines comprising a Modified Vaccinia Ankara (MVA) virus that, in turn, comprises a nucleotide sequence encoding the amino acid sequence of SEQ ID NO: 3 to thereby treat or prevent the prostate cancer.

Embodiment 2. The method of embodiment 1, further comprising administering the treatment regimen two or more times.
Embodiment 3. The method of embodiment 1 or 2, further comprising administering:

one or more vaccines comprising the GAd20 virus,

one or more vaccines comprising the MVA virus, or

one or more vaccines comprising the GAd20 virus and one or more vaccines comprising the MVA virus.

Embodiment 4. The method of embodiment 3, comprising administering two vaccines comprising the GAd20 virus and one vaccine comprising the MVA virus.
Embodiment 5. The method of embodiment 3, comprising administering one vaccine comprising the GAd20 virus and one vaccine comprising the MVA virus.
Embodiment 6. The method of embodiment 3, comprising administering three vaccines comprising the MVA virus.
Embodiment 7. The method of embodiment 3, comprising administering two vaccines comprising the MVA virus.
Embodiment 8. The method of any one of the previous embodiments, further comprising administering one or more vaccines comprising the MVA virus.
Embodiment 9. The method of any one of the previous embodiments, wherein each of the vaccines comprising the GAd20 virus comprises about 1×10⁹ viral particles (VP) to about 1 x 10¹³ VP of the GAd20 virus.
Embodiment 10. The method of any one of the previous embodiments, wherein each of the vaccines comprising the MVA virus comprises about 1×10⁶ infectious units (IFU) to about 1 x 10¹⁰ IFU of the MVA virus.
Embodiment 11. The method of any one of the previous embodiments, further comprising administering an anti-CTLA4 antibody.
Embodiment 12. The method of embodiment 11, comprising administering the anti-CTLA4 antibody with:

the vaccines comprising the GAd20 virus;

the vaccines comprising the MVA virus; or

both.

Embodiment 13. The method of embodiment 11 or 12, comprising administering 0.5 mg/kg to 5 mg/kg of the anti-CTLA4 antibody.
Embodiment 14. The method of any one of embodiments 1-10, further comprising administering an anti-PD-1 antibody.
Embodiment 15. The method of embodiment 14, comprising administering the anti-PD-1 antibody with:

the vaccines comprising the GAd20 virus;

the vaccines comprising the MVA virus; or

both.

Embodiment 16. The method of embodiment 14 or 15, comprising administering 0.5 mg/kg to 5 mg/kg of the anti-PD-1 antibody.
Embodiment 17. The method of any one of the previous embodiments, wherein each of the one or more GAd20 viruses comprise the nucleotide sequence of SEQ ID NO: 2.
Embodiment 18. The method of embodiment 17, wherein the nucleotide sequence further comprises an N-terminal TCE, a C-terminal His tag, or both.
Embodiment 19. The method of any one of the previous embodiments, wherein each of the one or more MVA viruses comprise the nucleotide sequence of SEQ ID NO: 4.
Embodiment 20. The method of embodiment 19, wherein the nucleotide sequence further comprises an N-terminal TCE, a C-terminal His tag, or both.
Embodiment 21. The method of any one of the previous embodiments, comprising administering a vaccine comprising the GAd20 virus at week 0 and about week 3 and administering a vaccine comprising the MVA virus at about week 9.
Embodiment 22. The method of embodiment 21, comprising administering a vaccine comprising the GAd20 virus at about week 15 and about week 18 and administering a vaccine comprising the MVA virus at about week 24.
Embodiment 23. The method of embodiment 21, comprising administering a vaccine comprising the GAd20 virus at about week 15 and administering a vaccine comprising the MVA virus at about week 24.
Embodiment 24. The method of embodiment 21, comprising administering a vaccine comprising the MVA virus at about week 15, about week 18, and about week 24.
Embodiment 25. The method of embodiment 21, comprising administering a vaccine comprising the MVA virus at about week 15 and about week 24.
Embodiment 26. The method of any one of the previous embodiments, comprising administering a vaccine comprising the MVA virus at about week 36, about week 48, and about week 60.
Embodiment 27. The method of any one of the previous embodiments, comprising administering a vaccine comprising 1×10¹¹ VP of the GAd20 virus at week 0 and about week 3 and administering a vaccine comprising 1×10⁸ IFU of the MVA virus at about week 9.
Embodiment 28. The method of embodiment 27, comprising administering a vaccine comprising 1×10¹¹ VP of the GAd20 virus at about week 15 and about week 18 and administering a vaccine comprising 1×10⁸ IFU of the MVA virus at about week 24.
Embodiment 29. The method of embodiment 27, comprising administering a vaccine comprising 1×10¹¹ VP of the GAd20 virus at about week 15 and administering a vaccine comprising 1 x 10⁸ IFU of the MVA virus at about week 24.
Embodiment 30. The method of embodiment 27, comprising administering a vaccine comprising 1×10⁸ IFU of the MVA virus at about week 15, about week 18, and about week 24.
Embodiment 31. The method of embodiment 27, comprising administering a vaccine comprising 1×10⁸ IFU of the MVA virus at about week 15 and about week 24.
Embodiment 32. The method of any one of the previous embodiments, comprising administering a vaccine comprising 1×10⁸ IFU of the MVA virus at about week 36, about week 48, and about week 60.
Embodiment 33. The method of embodiment 32, comprising administering one or more further vaccines comprising 1×10⁸ IFU of the MVA virus.
Embodiment 34. A method of treating or preventing prostate cancer in a subject, the method comprising administering to the subject:

a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;

a vaccine comprising 1×10⁸ infectious units (IFU) of an MVA virus at about week 9, wherein the MVA virus comprises a nucleotide sequence encoding the amino acid sequence of SEQ ID NO: 3;

a vaccine comprising 1×10¹¹ VP of the GAd20 virus at about week 15 and about week 18;

a vaccine comprising 1×10⁸ IFU of the MVA virus at about week 24; and

a vaccine comprising 1×10⁸ IFU of the MVA virus at about week 36, about week 48, and about week 60.

Embodiment 35. A method of treating or preventing prostate cancer in a subject, the method comprising administering to the subject:

a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;

a vaccine comprising 1×10⁸ infectious units (IFU) of an MVA virus at about week 9, wherein the MVA virus comprises a nucleotide sequence encoding the amino acid sequence of SEQ ID NO: 3;

a vaccine comprising 1×10¹¹ VP of the GAd20 virus at about week 15;

a vaccine comprising 1×10⁸ IFU of the MVA virus at about week 24; and

a vaccine comprising 1×10⁸ IFU of the MVA virus at about week 36, about week 48, and about week 60.

Embodiment 36. A method of treating or preventing prostate cancer in a subject, the method comprising administering to the subject:

a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;

a vaccine comprising 1×10⁸ infectious units (IFU) of an MVA virus at about week 9, wherein the MVA virus comprises a nucleotide sequence encoding the amino acid sequence of SEQ ID NO: 3;

a vaccine comprising 1×10⁸ IFU of the MVA virus at about week 15, about week 18, and about week 24; and

a vaccine comprising 1×10⁸ IFU of the MVA virus at about week 36, about week 48, and about week 60.

Embodiment 37. A method of treating or preventing prostate cancer in a subject, the method comprising administering to the subject:

a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;

a vaccine comprising 1×10⁸ infectious units (IFU) of an MVA virus at about week 9, wherein the MVA virus comprises a nucleotide sequence encoding the amino acid sequence of SEQ ID NO: 3;

a vaccine comprising 1×10⁸ IFU of the MVA virus at about week 15 and about week 24; and

a vaccine comprising 1×10⁸ IFU of the MVA virus at about week 36, about week 48, and about week 60.

Embodiment 38. The method of any one of embodiments 34-37, further comprising administering an anti-CTLA4 antibody.
Embodiment 39. The method of embodiment 38, comprising administering the anti-CTLA4 antibody with:

the vaccines comprising the GAd20 virus;

the vaccines comprising the MVA virus; or

both.

Embodiment 40. The method of embodiment 38 or 39, comprising administering 1 mg/kg to 3 mg/kg of the anti-CTLA4 antibody.
Embodiment 41. The method of any one of embodiments 34-37, further comprising administering an anti-PD-1 antibody.
Embodiment 42. The method of embodiment 41, comprising administering the anti-PD-1 antibody with:

the vaccines comprising the GAd20 virus;

the vaccines comprising the MVA virus; or

both.

Embodiment 43. The method of embodiment 41 or 42, comprising administering 1 mg/kg to 3 mg/kg of the anti-PD-1 antibody.
Embodiment 44. The method of any one of embodiments 34 to 43, wherein the GAd20 virus comprises the nucleotide sequence of SEQ ID NO: 2.
Embodiment 45. The method of embodiment 44, wherein the nucleotide sequence further comprises an N-terminal TCE, a C-terminal His tag, or both.
Embodiment 46. The method of any one of embodiments 34 to 43, wherein the MVA virus comprises the nucleotide sequence of SEQ ID NO: 4.
Embodiment 47. The method of embodiment 46, wherein the nucleotide sequence further comprises an N-terminal TCE, a C-terminal His tag, or both.
Embodiment 48. A method of treating or preventing prostate cancer in a subject, the method comprising administering to the subject:

1 mg/kg to 3 mg/kg of an anti-CTLA4 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;

1 mg/kg to 3 mg/kg of an anti-CTLA4 antibody and a vaccine comprising 1×10⁸ infectious units (IFU) of an MVA virus at about week 9, wherein the MVA virus comprises a nucleotide sequence encoding the amino acid sequence of SEQ ID NO: 3;

1 mg/kg to 3 mg/kg of an anti-CTLA4 antibody and a vaccine comprising 1×10¹¹ VP of the GAd20 virus at about week 15 and about week 18;

1 mg/kg to 3 mg/kg of an anti-CTLA4 antibody and a vaccine comprising 1×10⁸ IFU of the MVA virus at about week 24; and

1 mg/kg to 3 mg/kg of an anti-CTLA4 antibody and a vaccine comprising 1×10⁸ IFU of the MVA virus at about week 36, about week 48, and about week 60.

Embodiment 49. A method of treating or preventing prostate cancer in a subject, the method comprising administering to the subject:

1 mg/kg to 3 mg/kg of an anti-PD1 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;

1 mg/kg to 3 mg/kg of an anti-PD1 antibody and a vaccine comprising 1×10⁸ infectious units (IFU) of an MVA virus at about week 9, wherein the MVA virus comprises a nucleotide sequence encoding the amino acid sequence of SEQ ID NO: 3;

1 mg/kg to 3 mg/kg of an anti-PD1 antibody and a vaccine comprising 1×10¹¹ VP of the GAd20 virus at about week 15 and about week 18;

1 mg/kg to 3 mg/kg of an anti-PD1 antibody and a vaccine comprising 1×10⁸ IFU of the MVA virus at about week 24; and

1 mg/kg to 3 mg/kg of an anti-PD1 antibody and a vaccine comprising 1×10⁸ IFU of the MVA virus at about week 36, about week 48, and about week 60.

Embodiment 50. A method of treating or preventing prostate cancer in a subject, the method comprising administering to the subject:

1 mg/kg to 3 mg/kg of an anti-CTLA4 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;

1 mg/kg to 3 mg/kg of an anti-CTLA4 antibody and a vaccine comprising 1×10⁸ infectious units (IFU) of an MVA virus at about week 9, wherein the MVA virus comprises a nucleotide sequence encoding the amino acid sequence of SEQ ID NO: 3;

1 mg/kg to 3 mg/kg of an anti-CTLA4 antibody and a vaccine comprising 1×10¹¹ VP of the GAd20 virus at about week 15;

1 mg/kg to 3 mg/kg of an anti-CTLA4 antibody and a vaccine comprising 1×10⁸ IFU of the MVA virus at about week 24; and

1 mg/kg to 3 mg/kg of an anti-CTLA4 antibody and a vaccine comprising 1×10⁸ IFU of the MVA virus at about week 36, about week 48, and about week 60.

Embodiment 51. A method of treating or preventing prostate cancer in a subject, the method comprising administering to the subject:

1 mg/kg to 3 mg/kg of an anti-PD1 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;

1 mg/kg to 3 mg/kg of an anti-PD1 antibody and a vaccine comprising 1×10⁸ infectious units (IFU) of an MVA virus at about week 9, wherein the MVA virus comprises a nucleotide sequence encoding the amino acid sequence of SEQ ID NO: 3;

1 mg/kg to 3 mg/kg of an anti-PD1 antibody and a vaccine comprising 1×10¹¹ VP of the GAd20 virus at about week 15;

1 mg/kg to 3 mg/kg of an anti-PD1 antibody and a vaccine comprising 1×10⁸ IFU of the MVA virus at about week 24; and

1 mg/kg to 3 mg/kg of an anti-PD1 antibody and a vaccine comprising 1×10⁸ IFU of the MVA virus at about week 36, about week 48, and about week 60.

Embodiment 52. A method of treating or preventing prostate cancer in a subject, the method comprising administering to the subject:

1 mg/kg to 3 mg/kg of an anti-CTLA4 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;

1 mg/kg to 3 mg/kg of an anti-CTLA4 antibody and a vaccine comprising 1×10⁸ infectious units (IFU) of an MVA virus at about week 9, wherein the MVA virus comprises a nucleotide sequence encoding the amino acid sequence of SEQ ID NO: 3;

1 mg/kg to 3 mg/kg of an anti-CTLA4 antibody and a vaccine comprising 1×10⁸ IFU of the MVA virus at about week 15, about week 18, and about week 24; and

1 mg/kg to 3 mg/kg of an anti-CTLA4 antibody and a vaccine comprising 1×10⁸ IFU of the MVA virus at about week 36, about week 48, and about week 60.

Embodiment 53. A method of treating or preventing prostate cancer in a subject, the method comprising administering to the subject:

1 mg/kg to 3 mg/kg of an anti-PD1 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;

1 mg/kg to 3 mg/kg of an anti-PD1 antibody and a vaccine comprising 1×10⁸ infectious units (IFU) of an MVA virus at about week 9, wherein the MVA virus comprises a nucleotide sequence encoding the amino acid sequence of SEQ ID NO: 3;

1 mg/kg to 3 mg/kg of an anti-PD1 antibody and a vaccine comprising 1×10⁸ IFU of the MVA virus at about week 15, about week 18, and about week 24; and

1 mg/kg to 3 mg/kg of an anti-PD1 antibody and a vaccine comprising 1×10⁸ IFU of the MVA virus at about week 36, about week 48, and about week 60.

Embodiment 54. A method of treating or preventing prostate cancer in a subject, the method comprising administering to the subject:

1 mg/kg to 3 mg/kg of an anti-CTLA4 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;

1 mg/kg to 3 mg/kg of an anti-CTLA4 antibody and a vaccine comprising 1×10⁸ infectious units (IFU) of an MVA virus at about week 9, wherein the MVA virus comprises a nucleotide sequence encoding the amino acid sequence of SEQ ID NO: 3;

1 mg/kg to 3 mg/kg of an anti-CTLA4 antibody and a vaccine comprising 1×10⁸ IFU of the MVA virus at about week 15 and about week 24; and

1 mg/kg to 3 mg/kg of an anti-CTLA4 antibody and a vaccine comprising 1×10⁸ IFU of the MVA virus at about week 36, about week 48, and about week 60.

Embodiment 55. A method of treating or preventing prostate cancer in a subject, the method comprising administering to the subject:

1 mg/kg to 3 mg/kg of an anti-PD1 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;

1 mg/kg to 3 mg/kg of an anti-PD1 antibody and a vaccine comprising 1×10⁸ infectious units (IFU) of an MVA virus at about week 9, wherein the MVA virus comprises a nucleotide sequence encoding the amino acid sequence of SEQ ID NO: 3;

1 mg/kg to 3 mg/kg of an anti-PD1 antibody and a vaccine comprising 1×10⁸ IFU of the MVA virus at about week 15 and about week 24; and

1 mg/kg to 3 mg/kg of an anti-PD1 antibody and a vaccine comprising 1×10⁸ IFU of the MVA virus at about week 36, about week 48, and about week 60.

What is claimed:

1. A method of treating or preventing prostate cancer in a subject, the method comprising administering to the subject a treatment regimen comprising:
two or more vaccines comprising a great ape adenovirus serotype 20 (GAd20) virus that, in turn, comprises a nucleotide sequence encoding the amino acid sequence of SEQ ID NO: 1 and
one or more vaccines comprising a Modified Vaccinia Ankara (MVA) virus that, in turn, comprises a nucleotide sequence encoding the amino acid sequence of SEQ ID NO: 3 to thereby treat or prevent the prostate cancer.

2. The method of claim 1, further comprising administering the treatment regimen two or more times.

3. The method of claim 1, further comprising administering:
one or more vaccines comprising the GAd20 virus,
one or more vaccines comprising the MVA virus, or
one or more vaccines comprising the GAd20 virus and one or more vaccines comprising the MVA virus.

4. The method of claim 1, wherein each of the vaccines comprising the GAd20 virus comprises about 1×10⁹ viral particles (VP) to about 1×10¹³ VP of the GAd20 virus.

5. The method of claim 1, wherein each of the vaccines comprising the MVA virus comprises about 1×10⁶ infectious units (IFU) to about 1×10¹⁰ IFU of the MVA virus.

6. The method of claim 1, further comprising administering the anti-CTLA4 antibody with:
the vaccines comprising the GAd20 virus;
the vaccines comprising the MVA virus; or
both.

7. The method of claim 6, comprising administering 0.5 mg/kg to 5 mg/kg of the anti-CTLA4 antibody.

8. The method of claim 1, further comprising administering the anti-PD-1 antibody with:
the vaccines comprising the GAd20 virus;
the vaccines comprising the MVA virus; or
both.

9. The method of claim 8, comprising administering 0.5 mg/kg to 5 mg/kg of the anti-PD-1 antibody.

10. The method of claim 1, wherein each of the one or more GAd20 viruses comprise the nucleotide sequence of SEQ ID NO: 2.

11. The method of claim 10, wherein the nucleotide sequence further comprises an N-terminal TCE, a C-terminal His tag, or both.

12. The method of claim 1, wherein each of the one or more MVA viruses comprise the nucleotide sequence of SEQ ID NO: 4.

13. The method of claim 12, wherein the nucleotide sequence further comprises an N-terminal TCE, a C-terminal His tag, or both.

14. The method of claim 1, comprising administering a vaccine comprising the GAd20 virus at week 0, about week 3, about week 15, and week 18, and administering a vaccine comprising the MVA virus at about week 9, about week 24, about week 36, about week 48, and about week 60.

15. A method of treating or preventing prostate cancer in a subject, the method comprising administering to the subject:
a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;
a vaccine comprising 1×10⁸ infectious units (IFU) of an MVA virus at about week 9, wherein the MVA virus comprises a nucleotide sequence encoding the amino acid sequence of SEQ ID NO: 3;
a vaccine comprising 1×10¹¹ VP of the GAd20 virus at about week 15 and about week 18;
a vaccine comprising 1×10⁸ IFU of the MVA virus at about week 24; and
a vaccine comprising 1×10⁸ IFU of the MVA virus at about week 36, about week 48, and about week 60.

16. The method of claim 15, further comprising administering 1 mg/kg to 3 mg/kg of an anti-CTLA4 antibody with:
the vaccines comprising the GAd20 virus;
the vaccines comprising the MVA virus; or
both.

17. The method of claim 15, further comprising administering 1 mg/kg to 3 mg/kg of an anti-PD-1 antibody with:
the vaccines comprising the GAd20 virus;
the vaccines comprising the MVA virus; or
both.

18. The method of claim 15, wherein the GAd20 virus comprises the nucleotide sequence of SEQ ID NO: 2.

19. The method of claim 15, wherein the MVA virus comprises the nucleotide sequence of SEQ ID NO: 4.

20. A method of treating or preventing prostate cancer in a subject, the method comprising administering to the subject:
1 mg/kg to 3 mg/kg of an anti-CTLA4 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;
1 mg/kg to 3 mg/kg of an anti-CTLA4 antibody and a vaccine comprising 1×10⁸ infectious units (IFU) of an MVA virus at about week 9, wherein the MVA virus comprises a nucleotide sequence encoding the amino acid sequence of SEQ ID NO: 3;
1 mg/kg to 3 mg/kg of an anti-CTLA4 antibody and a vaccine comprising 1×10¹¹ VP of the GAd20 virus at about week 15 and about week 18;
1 mg/kg to 3 mg/kg of an anti-CTLA4 antibody and a vaccine comprising 1×10⁸ IFU of the MVA virus at about week 24; and
1 mg/kg to 3 mg/kg of an anti-CTLA4 antibody and a vaccine comprising 1×10⁸ IFU of the MVA virus at about week 36, about week 48, and about week 60.

21. A method of treating or preventing prostate cancer in a subject, the method comprising administering to the subject:
1 mg/kg to 3 mg/kg of an anti-PD1 antibody and a vaccine comprising 1×10¹¹ viral particles (VP) of a GAd20 virus at week 0 and about week 3, wherein the GAd20 virus comprises a nucleotide sequence encoding the amino acid sequence of SEQ ID NO: 1;
1 mg/kg to 3 mg/kg of an anti-PD1 antibody and a vaccine comprising 1×10⁸ infectious units (IFU) of an MVA virus at about week 9, wherein the MVA virus comprises a nucleotide sequence encoding the amino acid sequence of SEQ ID NO: 3;
1 mg/kg to 3 mg/kg of an anti-PD1 antibody and a vaccine comprising 1×10¹¹ VP of the GAd20 virus at about week 15 and about week 18;
1 mg/kg to 3 mg/kg of an anti-PD1 antibody and a vaccine comprising 1×10⁸ IFU of the MVA virus at about week 24; and
1 mg/kg to 3 mg/kg of an anti-PD1 antibody and a vaccine comprising 1×10⁸ IFU of the MVA virus at about week 36, about week 48, and about week 60.


SEQUENCE LISTING (94 sequences)

SEQ ID NO: 1 - protein, 1479 aa, synthetic construct
QNLQNGGGSRSSATLPGRRRRRWLRRRRQPISVAPAGPPRRPNQKPNPPGGARCVIMRPTWPGTSAFTKRSFAVTERIIDYWAQKEKGSSSFLRPSCDYWAQKEKISIPRTHLCLVLGVLSGHSGSRLYEAGMTLGGKILFFLFLLLPLSPFSLIFTEISCCTLSSEENEYLPRPEWQLQVPFRELKNVSVLEGLRQGRLGGPCSCHCPRPSQARLTPVDVAGPFLCLGDPGLFPPVKSSITGGKSTCSAPGPQSLPSTPFSTYPQWVILITELGMECTLGQVGAPSPRREEDGWRGGHSRFKADVPAPQGPCWGGQPGSAPSSAPPEQSLLDWKFEMSYTVGGPPPHVHARPRHWKTDRDGHSYTSKVNCLLLQDGFHGCVSITGAAGRRNLSIFLFLMLCKLEFHACKIQNKNCPDFKKFDGPCGERGGGRTARALWARGDSVLTPALDPQTPVRAPSLTRAAAAVHYKLIQQPISLFSITDRLHKTFSQLPSVHLCSITFQWGHPPIFCSTNDICVTANFCISVTFLKPCFLLHEASASQCHLFLQPQVGTPPPHTASARAPSGPPHPHESCPAGRRPARAAQTCARRQHGLPGCEEAGTARVPSLHLHLHQAALGAGRGRGWGEACAQVPPSRGVLRFLDLKVRYLHSQWQHYHRSGEAAGTPLWRPTRNVPFRELKNQRTAQGAPGIHHAASPVAANLCDPARHAQHTRIPCGAGQVRAGRGPEAGGGVLQPQRPAPEKPGCPCRRGQPRLHTVKMWRAVAMMVPDRQVHYDFGLGVPGDSTRRAVRRMNTFYEAGMTLGEKFRVGNCKHLKMTRPNSKMALNSEALSVVSECGASACDVSLIAMDSAFVQGKDWGVKKFIRRDFYAYKDFLWCFPFSLVFLQEIQICCHVSCLCCICCSTRICLGCLLELFLSRALRALHVLWNGFQLHCQTEYNQKLQVNQFSESKSLYHREKQLIAMDSAICEERGAAGSLISCETMPAILKLQKNCLLSLRTALTHNQDFSIYRLCCKRGSLCHASQARSPAFPKPVRPLPAPITRITPQLGGQSDSSQPLLTTGRPQGWQDQALRHTQQASPASCATITIPIHSAALGDHSGDPGPAWDTCPPLPLTTLIPRAPPPYGDSTARSWPSRCGPLGGNTTLQQLGEASQAPSGSLIPLRLPLLWEVRGQPDSFAALHSSLNELGEIARELHQFAFDLLIKSHFVQGKDWGLKKFIRRDFWGMELAASRRFSWDHHSAGGPPRVPSVRSGAAQVQPKDPLPLRTLAGCLARTAHLRPGAESLPQPQLHCTLWFQSSELSPTGAPWPSRRPTWRGTTVSPRTATSSARTCCGTKWPSSQEAALGLGSGLLRFSCGTAAIRKMHFSLKEHPPPPCPPEAFQRAAGEGGPGRGGARRGARVLQSPFCRAGAGEWLGHQSLRHVVGYGHLDTSGSSSSSSWPNSKMALNSLNSIDDAQLTRIAPPRSHCCFWEVNAP

SEQ ID NO: 2 - DNA, 4437 bp, synthetic construct
  source 1..4437: mol_type = other DNA
cagaacctgcagaacggcggaggctctagaagctctgctacacttcctggcaggcggcggagaagatggctgagaagaaggcggcagcctatctctgtggctcctgctggacctcctagacggcccaaccagaagcctaatcctcctggcggagccagatgcgtgatcatgaggcctacatggcctggcaccagcgccttcaccaagagaagctttgccgtgaccgagcggatcatcgactattgggctcaaaaagagaagggcagcagcagcttcctgcggcctagctgtgattattgggcccagaaagaaaagatcagcatccccagaacacacctgtgcctggtgctgggagtgctgtctggacactctggcagcagactgtatgaggccggcatgacactcggcggcaagatcctgttcttcctgttcctgctgctccctctgagccccttcagcctgatcttcaccgagatcagctgctgcaccctgagcagcgaggaaaacgagtacctgcctagacctgagtggcagctgcaggtccccttcagagagctgaagaacgtttccgtgctggaaggcctgagacagggcagacttggcggcccttgtagctgtcactgccccagacctagtcaggccagactgacacctgtggatgtggccggacctttcctgtgtctgggagatcctggcctgtttccacctgtgaagtccagcatcacaggcggcaagtccacatgttctgcccctggacctcagagcctgcctagcacacccttcagcacataccctcagtgggtcatcctgatcaccgaactcggcatggaatgcaccctgggacaagtgggagccccatctcctagaagagaagaggatggctggcgcggaggccactctagattcaaagctgatgtgcccgctcctcagggcccttgttggggaggacaacctggatctgccccatcttctgccccacctgaacagtccctgctggattggaagttcgagatgagctacaccgtcggcggacctccacctcatgttcatgccagacctcggcactggaaaaccgacagagatggccacagctacaccagcaaagtgaactgcctcctgctgcaggatggcttccacggctgtgtgtctattactggcgccgctggcagacggaacctgagcatctttctgtttctgatgctgtgcaagctcgagttccacgcctgcaagatccagaacaagaactgccccgacttcaagaagttcgacggcccttgcggagaaagaggcggaggcagaacagctagagccctttgggctagaggcgacagcgttctgacaccagctctggaccctcagacacctgttagggcccctagcctgacaagagctgccgccgctgtgcactacaagctgatccagcagccaatcagcctgttcagcatcaccgaccggctgcacaagacattcagccagctgccaagcgtgcacctgtgctccatcaccttccagtggggacaccctcctatcttttgctccaccaacgacatctgcgtgaccgccaacttctgtatcagcgtgaccttcctgaagccttgctttctgctgcacgaggccagcgcctctcagtgccacttgtttctgcagccccaagtgggcacacctcctccacatacagcctctgctagagcacctagcggccctccacatcctcacgaatcttgtcctgccggaagaaggcctgccagagccgctcaaacatgtgccagacgacagcacggactgcctggatgtgaagaggctggaacagccagagtgcctagcctgcacctccatctgcatcaggctgctcttggagccggaagaggtagaggatggggcgaagcttgtgctcaggtgccaccttctagaggcgtgctgagattcctggacctgaaagtgcgctacctgcacagccagtggcagcactatcacagatctggcgaagccgccggaacacccctttggaggccaacaagaaacgtgcccttccgggaactgaagaaccagagaacagctcagggcgctcctggaatccaccatgctgcttctccagtggccgccaacctgtgtgatcctgccagacatgcccagcacaccaggattccttgtggcgctggacaagtgcgcgctggaagaggacctgaagcaggcggaggtgttctgcaacctcaaagacccgctcctgagaagcctggctgcccttgcagaagaggacagcctagactgcacaccgtgaaaatgtggcgagccgtggccatgatggtgcccgatagacaggtccactacgactttggactgggcgtgccaggcgatagcactcggagagccgtcagacggatgaacaccttttacgaagccgggatgaccctgggcgagaagttcagagtgggcaactgcaagcacctgaagatgacccggcctaacagcaagatggccctgaatagcgaggccctgtctgtggtgtctgaatgtggcgcctctgcctgtgacgtgtccctgatcgctatggactccgcctttgtgcagggcaaagactggggcgtgaagaagttcatccggcgggacttctacgcctacaaggacttcctgtggtgcttccccttctctctggtgttcctgcaagagatccagatctgctgtcatgtgtcctgcctgtgctgcatctgctgtagcaccagaatctgcctgggctgtctgctggaactgttcctgagcagagccctgagagcactgcacgtgctgtggaacggattccagctgcactgccagaccgagtacaaccagaaactgcaagtgaaccagttcagcgagagcaagagcctgtaccaccgggaaaagcagctcattgccatggacagcgccatctgcgaagagagaggcgccgcaggatctctgatctcctgcgaaacaatgcccgccatcctgaagctgcagaagaattgcctcctaagcctgcgaaccgctctgacacacaaccaggacttcagcatctacagactgtgttgcaagcggggctccctgtgccatgcaagccaagctagaagccccgcctttcctaaacctgtgcgacctctgccagctccaatcaccagaattacccctcagctcggcggccagagcgattcatctcaacctctgctgaccaccggcagacctcaaggctggcaagaccaagctctgagacacacccagcaggctagccctgcctcttgtgccaccatcacaatccccatccactctgccgctctgggcgatcattctggcgatcctggaccagcctgggacacatgtcctccactgccactcacaacactgatccctagggctcctccaccttacggcgattctaccgctagaagctggcccagcagatgtggaccactcggaggcaacacaaccctccagcaactgggagaagcctctcaggctcctagcggctctctgatccctctcagactgcctctcctgtgggaagttcggggccagcctgattcttttgccgcactgcacagctccctgaacgagctgggagagatcgctagagagctgcaccagttcgccttcgacctgctgatcaagagccacttcgtgcaaggcaaggattggggcctcaaaaagtttatccgcagagacttctggggcatggaactggccgccagcagaagattcagctgggatcatcatagcgcaggcggcccacctagagtgccatctgttagaagcggagctgcccaggtgcagcctaaagatcctctgccactgagaacactggccggctgccttgctagaacagcccatcttagacctggcgccgagtctctgcctcagccacaactgcactgtaccctgtggttccagtccagcgagctgtctcctactggtgccccttggccatctagacgccctacttggagaggcaccaccgtgtcaccaagaaccgccacaagcagcgccagaacctgttgtggcacaaagtggccctccagccaagaagccgctctcggacttggaagcggactgctgaggttctcttgtggaaccgccgccattcggaagatgcactttagcctgaaagaacaccctccaccaccttgtcctccagaggctttccaaagagctgctggcgaaggcggacctggtagaggtggtgctagaagaggtgctagggtgctgcagagcccattctgtagagcaggcgcaggcgaatggctgggccatcagagtctgagacatgtcgtcggctacggccacctggatacaagcggaagcagctctagctccagctggcctaactcaaaaatggctctgaacagcctgaactccatcgacgacgcccagctgacaagaatcgcccctcctagatctcactgctgcttttgggaagtgaacgcccca

SEQ ID NO: 3 - protein, 1479 aa, synthetic construct
QNLQNGGGSRSSATLPGRRRRRWLRRRRQPISVAPAGPPRRPNQKPNPPGGARCVIMRPTWPGTSAFTGMECTLGQVGAPSPRREEDGWRGGHSRFKADVPAPQGPCWGGQPGSAPSSAPPEQSLLDDYWAQKEKISIPRTHLCWKFEMSYTVGGPPPHVHARPRHWKTDRDYWAQKEKGSSSFLRPSCVPFRELKNVSVLEGLRQGRLGGPCSCHCPRPSQARLTPVDVAGPFLCLGDPGLFPPVKSSITEISCCTLSSEENEYLPRPEWQLQYEAGMTLGGKILFFLFLLLPLSPFSLIFLVLGVLSGHSGSRLKRSFAVTERIITGGKSTCSAPGPQSLPSTPFSTYPQWVILITELDGHSYTSKVNCLLLQDGFHGCVSITGAAGRRNLSIFLFLMLCKLEFHACQWQHYHRSGEAAGTPLWRPTRNVAMMVPDRQVHYDFGLKIQNKNCPDVLRFLDLKVRYLHSVPFRELKNQRTAQGAPGIHHAASPVAANLCDPARHAQHTRIPCGAGQVRAGRGPEAGGGVLQPQRPAPEKPGCPCRRGQPRLHTVKMWRACHLFLQPQVGTPPPHTASARAPSGPPHPHESCPAGRRPARAAQTCARRQHGLPGCEEAGTARVPSLHLHLHQAALGAGRGRGWGEACAQVPPSRGHYKLIQQPISLFSITDRLHKTFSQLPSVHLCSITFQWGHPPIFCSTNDICVTANFCISVTFLKPCFLLHEASASQFKKFDGPCGERGGGRTARALWARGDSVLTPALDPQTPVRAPSLTRAAAAVGVPGDSTRRAVRRMNTFCGASACDVSLIAMDSACEERGAAGSLISCESLYHREKQLIAMDSAIFVQGKDWGVKKFIRRDFTMPAILKLQKNCLLSLNSKMALNSEALSVVSEYEAGMTLGEKFRVGNCKHLKMTRPTEYNQKLQVNQFSESKRTALTHNQDFSIYRLCCKRGSLCHASQARSPAFPKPVRPLPAPITRITPQLGGQSDSSQPLLTTGRPQGWQDQALRHTQQASPASCATITIPIHSAALGDHSGDPGPAWDTCPPLPLTTLIPRAPPPYGDSTARSWPSRCGPLGYAYKDFLWCFPFSLVFLQEIQICCHVSCLCCICCSTRICLGCLLELFLSRALRALHVLWNGFQLHCQGNTTLQQLGEASQAPSGSLIPLRLPLLWEVRGNSKMALNSLNSIDDAQLTRIAPPRSHCCFWEVNAPFVQGKDWGLKKFIRRDFEAFQRAAGEGGPGRGGARRGARVLQSPFCRAGAGEWLGHQSLRWGMELAASRRFSWDHHSAGGPPRVPSVRSGAAQVQPKDPLPLRTLAGCLARTAHLRPGAESLPQPQLHCTIARELHQFAFDLLIKSHKMHFSLKEHPPPPCPPHVVGYGHLDTSGSSSSSSWPQPDSFAALHSSLNELGELWFQSSELSPTGAPWPSRRPTWRGTTVSPRTATSSARTCCGTKWPSSQEAALGLGSGLLRFSCGTAAIR

SEQ ID NO: 4 - DNA, 4437 bp, synthetic construct
  source 1..4437: mol_type = other DNA
cagaacctgcagaacggcggaggctctagaagctctgctacacttcctggcaggcggcggagaagatggctgagaagaaggcggcagcctatctctgtggctcctgctggacctcctagacggcccaaccagaagcctaatcctcctggcggagccagatgcgtgatcatgaggcctacatggcctggcaccagcgcctttaccggcatggaatgtacactgggccaagtgggagccccatctcctagaagagaagaggatggctggcgcggaggccactctagattcaaagctgatgtgcccgctcctcagggcccttgttggggaggacaacctggatctgccccatcttctgccccacctgaacagagcctgctggatgactattgggctcaaaaagagaagatcagcatccccagaacacacctgtgctggaagttcgagatgagctacaccgttggcggccctccaccacatgttcacgccagacctagacactggaaaaccgacagagattattgggcccagaaagaaaagggcagcagcagcttcctgcggcctagctgtgtgcccttccgggaactgaagaacgtgtccgttctggaaggcctgaggcagggcagacttggcggaccttgtagctgccactgtcctagaccaagccaggccagactgacccctgtggatgtggctggcccatttctgtgtctgggcgaccctggactgttccctccagtgaagtctagcatcaccgagatcagctgctgcaccctgagcagcgaggaaaacgagtacctgcctagacctgaatggcagctgcagtacgaggccggcatgacactcggaggcaagatcctgttcttcctgttcctgctgctccctctgagccccttcagcctgatctttctggtgctgggcgtgctgtctggccactctggaagcagactgaagagaagcttcgccgtgaccgagcggatcatcacaggcggcaagagcacatgttctgcccctggacctcagtctctgcccagcacacccttcagcacataccctcagtgggtcatcctgatcaccgagctggatggccacagctacaccagcaaagtgaactgcctcctgctgcaggatggcttccacggctgtgtgtctattactggcgccgctggcagacggaacctgagcatctttctgtttctgatgctgtgcaagctcgagttccacgcctgccaatggcagcactaccacagatctggcgaagccgctggaaccccactttggaggcctaccagaaacgtggccatgatggtgcccgacagacaggtgcactacgacttcggcctgaagatccagaacaagaactgccccgacgtgctgcggttcctggatctcaaagtgcgctacctgcacagcgtgcccttcagagagctgaaaaaccagagaacagcccagggcgctcctggaatccatcatgctgcttctccagtggccgccaatctgtgcgatcctgccagacatgcccagcataccaggattccttgtggcgctggacaagtgcgcgctggaagaggacctgaagctggtggcggagttctgcagcctcaaagacctgctcctgagaagcctggctgcccctgtagaagaggacagcctagactgcacaccgtgaagatgtggcgggcctgccacttgtttctccagccacaagtgggcacccctccacctcatacagcctctgctagagcacctagcggcccacctcatcctcacgaatcttgtcctgccggaagaaggcctgccagagccgctcaaacatgtgccagacgacagcacggactgcccggatgtgaagaagccggaacagccagagtgcctagcctgcaccttcatctgcatcaggccgctcttggagccggaagaggtagaggatggggagaagcttgtgcccaggtgccaccttctagaggccactacaagctgatccagcagccaatcagcctgttctccatcaccgaccggctgcacaagacattcagccagctgccttccgtgcatctgtgcagcatcaccttccagtggggacaccctcctatcttttgctccaccaacgacatctgcgtgaccgccaacttctgtatcagcgtgaccttcctgaagccttgctttctgctgcacgaggcctccgccagccagtttaagaagtttgacggcccctgcggcgagagaggcggaggaagaactgcaagagccctttgggccagaggcgactctgttctgacaccagctctggaccctcagacacctgttagggcccctagcctgacaagagctgccgctgctgttggagtgcctggcgattctactagaagggccgtgcggcggatgaacaccttttgtggcgcatctgcctgcgacgtgtccctgatcgctatggatagcgcctgcgaggaaagaggcgcagccggatctctgatctcttgcgagagcctgtaccaccgggaaaagcagctcattgccatggacagcgccatcttcgtgcagggcaaagactggggcgtgaagaagttcatccggcgggactttaccatgcctgccattctgaagctgcagaagaattgtcttctaagcctgaacagcaagatggccctgaatagcgaggccctgtctgtggtgtccgagtatgaggctggaatgaccctgggcgagaagttcagagtgggcaactgcaagcacctgaagatgacccggcctaccgagtacaaccagaaactgcaagtgaaccagttcagcgagagcaagcggaccgctctgacccacaaccaggacttcagcatctaccggctgtgctgcaagaggggctctctgtgtcatgctagccaggctagaagccccgcctttcctaagcctgtcagacctctgcctgctcctatcaccagaatcacccctcagctcggcggccagtctgattcatctcagccactgctgaccaccggcagacctcaaggatggcaagaccaggctctgagacacacacagcaggctagcccagcctcttgcgccaccatcacaataccaatacattctgccgctctgggcgatcacagcggagatcctggacctgcctgggatacttgtcctcctctgcccctaactacactgatccctagggctcctccaccttacggcgatagcacagccagatcctggcctagcagatgtggccctctgggctacgcctacaaggacttcctgtggtgcttccccttctctctggtgttcctgcaagaaatccagatctgctgtcacgtgtcctgcctgtgctgtatctgctgtagcacccggatctgtctgggctgtctgctggaactgttcctgagcagagccctgagagcactgcacgtgctgtggaacggattccagctgcactgccagggcaacaccacactgcaacagctgggagaagcctctcaggccccaagcggttctctgatccctctcagactgcccctcctgtgggaagtgcggggcaattctaagatggctctcaacagcctgaactccatcgacgacgcccagctgacaagaatcgcccctccaagaagccactgttgcttttgggaagtgaacgccccttttgtgcagggtaaagattggggcctcaaaaagtttatcagacgggacttcgaggctttccagagagcagctggcgaaggcggacctggcagaggtggtgctagaagaggtgctagagtgctgcagagcccattctgtagagctggcgctggcgaatggctgggccaccaatctcttagatggggaatggaactggccgctagcaggcggtttagctgggatcatcattctgccggcggacctccaagagtgccaagcgttagaagcggagcagcccaggtccagcctaaagatccactgccactgagaacactggccggctgccttgccagaacagctcatcttagacctggcgccgaaagcctgcctcaacctcagctgcattgcacaatcgccagagaactgcaccagttcgccttcgacctgctgatcaagagccacaagatgcacttctcactgaaagagcacccgccaccgccgtgcccaccgcacgttgtcggctatggccacctggatacaagcggctcctctagcagtagctcctggcctcagcctgacagctttgctgccctgcatagctccctgaatgagctgggcgaactgtggttccagtccagcgaactgtctcctactggcgctccatggccaagcagaaggcctacttggagaggcaccaccgtgtctccaagaaccgctacaagcagcgccagaacctgttgcggcacaaaatggccctccagccaagaagctgccctcggacttggaagcggactgctgagattcagctgtggcacagccgccatcaga

SEQ ID NO: 5 - protein, 28 aa, synthetic construct
MGQKEQIHTLQKNSERMSKQLTRSSQAV

SEQ ID NO: 6 - DNA, 84 bp, synthetic construct
  source 1..84: mol_type = other DNA
atgggccagaaagagcagatccacacactgcagaaaaacagcgagcggatgagcaagcagctgaccagatcttctcaggccgtg

SEQ ID NO: 7 - protein, 7 aa, synthetic construct
SHHHHHH

SEQ ID NO: 8 - DNA, 21 bp, synthetic construct
  source 1..21: mol_type = other DNA
agccatcaccatcaccaccat

SEQ ID NO: 9 - protein, 1514 aa, synthetic construct
MGQKEQIHTLQKNSERMSKQLTRSSQAVQNLQNGGGSRSSATLPGRRRRRWLRRRRQPISVAPAGPPRRPNQKPNPPGGARCVIMRPTWPGTSAFTKRSFAVTERIIDYWAQKEKGSSSFLRPSCDYWAQKEKISIPRTHLCLVLGVLSGHSGSRLYEAGMTLGGKILFFLFLLLPLSPFSLIFTEISCCTLSSEENEYLPRPEWQLQVPFRELKNVSVLEGLRQGRLGGPCSCHCPRPSQARLTPVDVAGPFLCLGDPGLFPPVKSSITGGKSTCSAPGPQSLPSTPFSTYPQWVILITELGMECTLGQVGAPSPRREEDGWRGGHSRFKADVPAPQGPCWGGQPGSAPSSAPPEQSLLDWKFEMSYTVGGPPPHVHARPRHWKTDRDGHSYTSKVNCLLLQDGFHGCVSITGAAGRRNLSIFLFLMLCKLEFHACKIQNKNCPDFKKFDGPCGERGGGRTARALWARGDSVLTPALDPQTPVRAPSLTRAAAAVHYKLIQQPISLFSITDRLHKTFSQLPSVHLCSITFQWGHPPIFCSTNDICVTANFCISVTFLKPCFLLHEASASQCHLFLQPQVGTPPPHTASARAPSGPPHPHESCPAGRRPARAAQTCARRQHGLPGCEEAGTARVPSLHLHLHQAALGAGRGRGWGEACAQVPPSRGVLRFLDLKVRYLHSQWQHYHRSGEAAGTPLWRPTRNVPFRELKNQRTAQGAPGIHHAASPVAANLCDPARHAQHTRIPCGAGQVRAGRGPEAGGGVLQPQRPAPEKPGCPCRRGQPRLHTVKMWRAVAMMVPDRQVHYDFGLGVPGDSTRRAVRRMNTFYEAGMTLGEKFRVGNCKHLKMTRPNSKMALNSEALSVVSECGASACDVSLIAMDSAFVQGKDWGVKKFIRRDFYAYKDFLWCFPFSLVFLQEIQICCHVSCLCCICCSTRICLGCLLELFLSRALRALHVLWNGFQLHCQTEYNQKLQVNQFSESKSLYHREKQLIAMDSAICEERGAAGSLISCETMPAILKLQKNCLLSLRTALTHNQDFSIYRLCCKRGSLCHASQARSPAFPKPVRPLPAPITRITPQLGGQSDSSQPLLTTGRPQGWQDQALRHTQQASPASCATITIPIHSAALGDHSGDPGPAWDTCPPLPLTTLIPRAPPPYGDSTARSWPSRCGPLGGNTTLQQLGEASQAPSGSLIPLRLPLLWEVRGQPDSFAALHSSLNELGEIARELHQFAFDLLIKSHFVQGKDWGLKKFIRRDFWGMELAASRRFSWDHHSAGGPPRVPSVRSGAAQVQPKDPLPLRTLAGCLARTAHLRPGAESLPQPQLHCTLWFQSSELSPTGAPWPSRRPTWRGTTVSPRTATSSARTCCGTKWPSSQEAALGLGSGLLRFSCGTAAIRKMHFSLKEHPPPPCPPEAFQRAAGEGGPGRGGARRGARVLQSPFCRAGAGEWLGHQSLRHVVGYGHLDTSGSSSSSSWPNSKMALNSLNSIDDAQLTRIAPPRSHCCFWEVNAPSHHHHHH

SEQ ID NO: 10 - DNA, 4542 bp, synthetic construct
  source 1..4542: mol_type = other DNA
atgggccagaaagagcagatccacacactgcagaaaaacagcgagcggatgagcaagcagctgaccagatcttctcaggccgtgcagaacctgcagaacggcggaggctctagaagctctgctacacttcctggcaggcggcggagaagatggctgagaagaaggcggcagcctatctctgtggctcctgctggacctcctagacggcccaaccagaagcctaatcctcctggcggagccagatgcgtgatcatgaggcctacatggcctggcaccagcgccttcaccaagagaagctttgccgtgaccgagcggatcatcgactattgggctcaaaaagagaagggcagcagcagcttcctgcggcctagctgtgattattgggcccagaaagaaaagatcagcatccccagaacacacctgtgcctggtgctgggagtgctgtctggacactctggcagcagactgtatgaggccggcatgacactcggcggcaagatcctgttcttcctgttcctgctgctccctctgagccccttcagcctgatcttcaccgagatcagctgctgcaccctgagcagcgaggaaaacgagtacctgcctagacctgagtggcagctgcaggtccccttcagagagctgaagaacgtttccgtgctggaaggcctgagacagggcagacttggcggcccttgtagctgtcactgccccagacctagtcaggccagactgacacctgtggatgtggccggacctttcctgtgtctgggagatcctggcctgtttccacctgtgaagtccagcatcacaggcggcaagtccacatgttctgcccctggacctcagagcctgcctagcacacccttcagcacataccctcagtgggtcatcctgatcaccgaactcggcatggaatgcaccctgggacaagtgggagccccatctcctagaagagaagaggatggctggcgcggaggccactctagattcaaagctgatgtgcccgctcctcagggcccttgttggggaggacaacctggatctgccccatcttctgccccacctgaacagtccctgctggattggaagttcgagatgagctacaccgtcggcggacctccacctcatgttcatgccagacctcggcactggaaaaccgacagagatggccacagctacaccagcaaagtgaactgcctcctgctgcaggatggcttccacggctgtgtgtctattactggcgccgctggcagacggaacctgagcatctttctgtttctgatgctgtgcaagctcgagttccacgcctgcaagatccagaacaagaactgccccgacttcaagaagttcgacggcccttgcggagaaagaggcggaggcagaacagctagagccctttgggctagaggcgacagcgttctgacaccagctctggaccctcagacacctgttagggcccctagcctgacaagagctgccgccgctgtgcactacaagctgatccagcagccaatcagcctgttcagcatcaccgaccggctgcacaagacattcagccagctgccaagcgtgcacctgtgctccatcaccttccagtggggacaccctcctatcttttgctccaccaacgacatctgcgtgaccgccaacttctgtatcagcgtgaccttcctgaagccttgctttctgctgcacgaggccagcgcctctcagtgccacttgtttctgcagccccaagtgggcacacctcctccacatacagcctctgctagagcacctagcggccctccacatcctcacgaatcttgtcctgccggaagaaggcctgccagagccgctcaaacatgtgccagacgacagcacggactgcctggatgtgaagaggctggaacagccagagtgcctagcctgcacctccatctgcatcaggctgctcttggagccggaagaggtagaggatggggcgaagcttgtgctcaggtgccaccttctagaggcgtgctgagattcctggacctgaaagtgcgctacctgcacagccagtggcagcactatcacagatctggcgaagccgccggaacacccctttggaggccaacaagaaacgtgcccttccgggaactgaagaaccagagaacagctcagggcgctcctggaatccaccatgctgcttctccagtggccgccaacctgtgtgatcctgccagacatgcccagcacaccaggattccttgtggcgctggacaagtgcgcgctggaagaggacctgaagcaggcggaggtgttctgcaacctcaaagacccgctcctgagaagcctggctgcccttgcagaagaggacagcctagactgcacaccgtgaaaatgtggcgagccgtggccatgatggtgcccgatagacaggtccactacgactttggactgggcgtgccaggcgatagcactcggagagccgtcagacggatgaacaccttttacgaagccgggatgaccctgggcgagaagttcagagtgggcaactgcaagcacctgaagatgacccggcctaacagcaagatggccctgaatagcgaggccctgtctgtggtgtctgaatgtggcgcctctgcctgtgacgtgtccctgatcgctatggactccgcctttgtgcagggcaaagactggggcgtgaagaagttcatccggcgggacttctacgcctacaaggacttcctgtggtgcttccccttctctctggtgttcctgcaagagatccagatctgctgtcatgtgtcctgcctgtgctgcatctgctgtagcaccagaatctgcctgggctgtctgctggaactgttcctgagcagagccctgagagcactgcacgtgctgtggaacggattccagctgcactgccagaccgagtacaaccagaaactgcaagtgaaccagttcagcgagagcaagagcctgtaccaccgggaaaagcagctcattgccatggacagcgccatctgcgaagagagaggcgccgcaggatctctgatctcctgcgaaacaatgcccgccatcctgaagctgcagaagaattgcctcctaagcctgcgaaccgctctgacacacaaccaggacttcagcatctacagactgtgttgcaagcggggctccctgtgccatgcaagccaagctagaagccccgcctttcctaaacctgtgcgacctctgccagctccaatcaccagaattacccctcagctcggcggccagagcgattcatctcaacctctgctgaccaccggcagacctcaaggctggcaagaccaagctctgagacacacccagcaggctagccctgcctcttgtgccaccatcacaatccccatccactctgccgctctgggcgatcattctggcgatcctggaccagcctgggacacatgtcctccactgccactcacaacactgatccctagggctcctccaccttacggcgattctaccgctagaagctggcccagcagatgtggaccactcggaggcaacacaaccctccagcaactgggagaagcctctcaggctcctagcggctctctgatccctctcagactgcctctcctgtgggaagttcggggccagcctgattcttttgccgcactgcacagctccctgaacgagctgggagagatcgctagagagctgcaccagttcgccttcgacctgctgatcaagagccacttcgtgcaaggcaaggattggggcctcaaaaagtttatccgcagagacttctggggcatggaactggccgccagcagaagattcagctgggatcatcatagcgcaggcggcccacctagagtgccatctgttagaagcggagctgcccaggtgcagcctaaagatcctctgccactgagaacactggccggctgccttgctagaacagcccatcttagacctggcgccgagtctctgcctcagccacaactgcactgtaccctgtggttccagtccagcgagctgtctcctactggtgccccttggccatctagacgccctacttggagaggcaccaccgtgtcaccaagaaccgccacaagcagcgccagaacctgttgtggcacaaagtggccctccagccaagaagccgctctcggacttggaagcggactgctgaggttctcttgtggaaccgccgccattcggaagatgcactttagcctgaaagaacaccctccaccaccttgtcctccagaggctttccaaagagctgctggcgaaggcggacctggtagaggtggtgctagaagaggtgctagggtgctgcagagcccattctgtagagcaggcgcaggcgaatggctgggccatcagagtctgagacatgtcgtcggctacggccacctggatacaagcggaagcagctctagctccagctggcctaactcaaaaatggctctgaacagcctgaactccatcgacgacgcccagctgacaagaatcgcccctcctagatctcactgctgcttttgggaagtgaacgccccaagccatcaccatcaccaccat

SEQ ID NO: 11 - protein, 1514 aa, synthetic construct
MGQKEQIHTLQKNSERMSKQLTRSSQAVQNLQNGGGSRSSATLPGRRRRRWLRRRRQPISVAPAGPPRRPNQKPNPPGGARCVIMRPTWPGTSAFTGMECTLGQVGAPSPRREEDGWRGGHSRFKADVPAPQGPCWGGQPGSAPSSAPPEQSLLDDYWAQKEKISIPRTHLCWKFEMSYTVGGPPPHVHARPRHWKTDRDYWAQKEKGSSSFLRPSCVPFRELKNVSVLEGLRQGRLGGPCSCHCPRPSQARLTPVDVAGPFLCLGDPGLFPPVKSSITEISCCTLSSEENEYLPRPEWQLQYEAGMTLGGKILFFLFLLLPLSPFSLIFLVLGVLSGHSGSRLKRSFAVTERIITGGKSTCSAPGPQSLPSTPFSTYPQWVILITELDGHSYTSKVNCLLLQDGFHGCVSITGAAGRRNLSIFLFLMLCKLEFHACQWQHYHRSGEAAGTPLWRPTRNVAMMVPDRQVHYDFGLKIQNKNCPDVLRFLDLKVRYLHSVPFRELKNQRTAQGAPGIHHAASPVAANLCDPARHAQHTRIPCGAGQVRAGRGPEAGGGVLQPQRPAPEKPGCPCRRGQPRLHTVKMWRACHLFLQPQVGTPPPHTASARAPSGPPHPHESCPAGRRPARAAQTCARRQHGLPGCEEAGTARVPSLHLHLHQAALGAGRGRGWGEACAQVPPSRGHYKLIQQPISLFSITDRLHKTFSQLPSVHLCSITFQWGHPPIFCSTNDICVTANFCISVTFLKPCFLLHEASASQFKKFDGPCGERGGGRTARALWARGDSVLTPALDPQTPVRAPSLTRAAAAVGVPGDSTRRAVRRMNTFCGASACDVSLIAMDSACEERGAAGSLISCESLYHREKQLIAMDSAIFVQGKDWGVKKFIRRDFTMPAILKLQKNCLLSLNSKMALNSEALSVVSEYEAGMTLGEKFRVGNCKHLKMTRPTEYNQKLQVNQFSESKRTALTHNQDFSIYRLCCKRGSLCHASQARSPAFPKPVRPLPAPITRITPQLGGQSDSSQPLLTTGRPQGWQDQALRHTQQASPASCATITIPIHSAALGDHSGDPGPAWDTCPPLPLTTLIPRAPPPYGDSTARSWPSRCGPLGYAYKDFLWCFPFSLVFLQEIQICCHVSCLCCICCSTRICLGCLLELFLSRALRALHVLWNGFQLHCQGNTTLQQLGEASQAPSGSLIPLRLPLLWEVRGNSKMALNSLNSIDDAQLTRIAPPRSHCCFWEVNAPFVQGKDWGLKKFIRRDFEAFQRAAGEGGPGRGGARRGARVLQSPFCRAGAGEWLGHQSLRWGMELAASRRFSWDHHSAGGPPRVPSVRSGAAQVQPKDPLPLRTLAGCLARTAHLRPGAESLPQPQLHCTIARELHQFAFDLLIKSHKMHFSLKEHPPPPCPPHVVGYGHLDTSGSSSSSSWPQPDSFAALHSSLNELGELWFQSSELSPTGAPWPSRRPTWRGTTVSPRTATSSARTCCGTKWPSSQEAALGLGSGLLRFSCGTAAIRSHHHHHH

SEQ ID NO: 12 - DNA, 4542 bp, synthetic construct
  source 1..4542: mol_type = other DNA
atgggccagaaagagcagatccacacactgcagaaaaacagcgagcggatgagcaagcagctgaccagatcttctcaggccgtgcagaacctgcagaacggcggaggctctagaagctctgctacacttcctggcaggcggcggagaagatggctgagaagaaggcggcagcctatctctgtggctcctgctggacctcctagacggcccaaccagaagcctaatcctcctggcggagccagatgcgtgatcatgaggcctacatggcctggcaccagcgcctttaccggcatggaatgtacactgggccaagtgggagccccatctcctagaagagaagaggatggctggcgcggaggccactctagattcaaagctgatgtgcccgctcctcagggcccttgttggggaggacaacctggatctgccccatcttctgccccacctgaacagagcctgctggatgactattgggctcaaaaagagaagatcagcatccccagaacacacctgtgctggaagttcgagatgagctacaccgttggcggccctccaccacatgttcacgccagacctagacactggaaaaccgacagagattattgggcccagaaagaaaagggcagcagcagcttcctgcggcctagctgtgtgcccttccgggaactgaagaacgtgtccgttctggaaggcctgaggcagggcagacttggcggaccttgtagctgccactgtcctagaccaagccaggccagactgacccctgtggatgtggctggcccatttctgtgtctgggcgaccctggactgttccctccagtgaagtctagcatcaccgagatcagctgctgcaccctgagcagcgaggaaaacgagtacctgcctagacctgaatggcagctgcagtacgaggccggcatgacactcggaggcaagatcctgttcttcctgttcctgctgctccctctgagccccttcagcctgatctttctggtgctgggcgtgctgtctggccactctggaagcagactgaagagaagcttcgccgtgaccgagcggatcatcacaggcggcaagagcacatgttctgcccctggacctcagtctctgcccagcacacccttcagcacataccctcagtgggtcatcctgatcaccgagctggatggccacagctacaccagcaaagtgaactgcctcctgctgcaggatggcttccacggctgtgtgtctattactggcgccgctggcagacggaacctgagcatctttctgtttctgatgctgtgcaagctcgagttccacgcctgccaatggcagcactaccacagatctggcgaagccgctggaaccccactttggaggcctaccagaaacgtggccatgatggtgcccgacagacaggtgcactacgacttcggcctgaagatccagaacaagaactgccccgacgtgctgcggttcctggatctcaaagtgcgctacctgcacagcgtgcccttcagagagctgaaaaaccagagaacagcccagggcgctcctggaatccatcatgctgcttctccagtggccgccaatctgtgcgatcctgccagacatgcccagcataccaggattccttgtggcgctggacaagtgcgcgctggaagaggacctgaagctggtggcggagttctgcagcctcaaagacctgctcctgagaagcctggctgcccctgtagaagaggacagcctagactgcacaccgtgaagatgtggcgggcctgccacttgtttctccagccacaagtgggcacccctccacctcatacagcctctgctagagcacctagcggcccacctcatcctcacgaatcttgtcctgccggaagaaggcctgccagagccgctcaaacatgtgccagacgacagcacggactgcccggatgtgaagaagccggaacagccagagtgcctagcctgcaccttcatctgcatcaggccgctcttggagccggaagaggtagaggatggggagaagcttgtgcccaggtgccaccttctagaggccactacaagctgatccagcagccaatcagcctgttctccatcaccgaccggctgcacaagacattcagccagctgccttccgtgcatctgtgcagcatcaccttccagtggggacaccctcctatcttttgctccaccaacgacatctgcgtgaccgccaacttctgtatcagcgtgaccttcctgaagccttgctttctgctgcacgaggcctccgccagccagtttaagaagtttgacggcccctgcggcgagagaggcggaggaagaactgcaagagccctttgggccagaggcgactctgttctgacaccagctctggaccctcagacacctgttagggcccctagcctgacaagagctgccgctgctgttggagtgcctggcgattctactagaagggccgtgcggcggatgaacaccttttgtggcgcatctgcctgcgacgtgtccctgatcgctatggatagcgcctgcgaggaaagaggcgcagccggatctctgatctcttgcgagagcctgtaccaccgggaaaagcagctcattgccatggacagcgccatcttcgtgcagggcaaagactggggcgtgaagaagttcatccggcgggactttaccatgcctgccattctgaagctgcagaagaattgtcttctaagcctgaacagcaagatggccctgaatagcgaggccctgtctgtggtgtccgagtatgaggctggaatgaccctgggcgagaagttcagagtgggcaactgcaagcacctgaagatgacccggcctaccgagtacaaccagaaactgcaagtgaaccagttcagcgagagcaagcggaccgctctgacccacaaccaggacttcagcatctaccggctgtgctgcaagaggggctctctgtgtcatgctagccaggctagaagccccgcctttcctaagcctgtcagacctctgcctgctcctatcaccagaatcacccctcagctcggcggccagtctgattcatctcagccactgctgaccaccggcagacctcaaggatggcaagaccaggctctgagacacacacagcaggctagcccagcctcttgcgccaccatcacaataccaatacattctgccgctctgggcgatcacagcggagatcctggacctgcctgggatacttgtcctcctctgcccctaactacactgatccctagggctcctccaccttacggcgatagcacagccagatcctggcctagcagatgtggccctctgggctacgcctacaaggacttcctgtggtgcttccccttctctctggtgttcctgcaagaaatccagatctgctgtcacgtgtcctgcctgtgctgtatctgctgtagcacccggatctgtctgggctgtctgctggaactgttcctgagcagagccctgagagcactgcacgtgctgtggaacggattccagctgcactgccagggcaacaccacactgcaacagctgggagaagcctctcaggccccaagcggttctctgatccctctcagactgcccctcctgtgggaagtgcggggcaattctaagatggctctcaacagcctgaactccatcgacgacgcccagctgacaagaatcgcccctccaagaagccactgttgcttttgggaagtgaacgccccttttgtgcagggtaaagattggggcctcaaaaagtttatcagacgggacttcgaggctttccagagagcagctggcgaaggcggacctggcagaggtggtgctagaagaggtgctagagtgctgcagagcccattctgtagagctggcgctggcgaatggctgggccaccaatctcttagatggggaatggaactggccgctagcaggcggtttagctgggatcatcattctgccggcggacctccaagagtgccaagcgttagaagcggagcagcccaggtccagcctaaagatccactgccactgagaacactggccggctgccttgccagaacagctcatcttagacctggcgccgaaagcctgcctcaacctcagctgcattgcacaatcgccagagaactgcaccagttcgccttcgacctgctgatcaagagccacaagatgcacttctcactgaaagagcacccgccaccgccgtgcccaccgcacgttgtcggctatggccacctggatacaagcggctcctctagcagtagctcctggcctcagcctgacagctttgctgccctgcatagctccctgaatgagctgggcgaactgtggttccagtccagcgaactgtctcctactggcgctccatggccaagcagaaggcctacttggagaggcaccaccgtgtctccaagaaccgctacaagcagcgccagaacctgttgcggcacaaaatggccctccagccaagaagctgccctcggacttggaagcggactgctgagattcagctgtggcacagccgccatcagaagccatcaccatcaccaccat

SEQ ID NO: 13 - protein, 68 aa, synthetic construct
QNLQNGGGSRSSATLPGRRRRRWLRRRRQPISVAPAGPPRRPNQKPNPPGGARCVIMRPTWPGTSAFT

SEQ ID NO: 14 - DNA, 204 bp, synthetic construct
  source 1..204: mol_type = other DNA
cagaacctgcagaacggcggaggctctagaagctctgctacacttcctggcaggcggcggagaagatggctgagaagaaggcggcagcctatctctgtggctcctgctggacctcctagacggcccaaccagaagcctaatcctcctggcggagccagatgcgtgatcatgaggcctacatggcctggcaccagcgccttcacc

SEQ ID NO: 15 - protein, 11 aa, synthetic construct
KRSFAVTERII

SEQ ID NO: 16 - DNA, 33 bp, synthetic construct
  source 1..33: mol_type = other DNA
aagagaagctttgccgtgaccgagcggatcatc

SEQ ID NO: 17 - protein, 18 aa, synthetic construct
DYWAQKEKGSSSFLRPSC

SEQ ID NO: 18 - DNA, 54 bp, synthetic construct
  source 1..54: mol_type = other DNA
gactattgggctcaaaaagagaagggcagcagcagcttcctgcggcctagctgt

SEQ ID NO: 19 - protein, 17 aa, synthetic construct
DYWAQKEKISIPRTHLC

SEQ ID NO: 20 - DNA, 51 bp, synthetic construct
  source 1..51: mol_type = other DNA
gattattgggcccagaaagaaaagatcagcatccccagaacacacctgtgc

SEQ ID NO: 21 - protein, 14 aa, synthetic construct
LVLGVLSGHSGSRL

SEQ ID NO: 22 - DNA, 42 bp, synthetic construct
  source 1..42: mol_type = other DNA
ctggtgctgggagtgctgtctggacactctggcagcagactg

SEQ ID NO: 23 - protein, 28 aa, synthetic construct
YEAGMTLGGKILFFLFLLLPLSPFSLIF

SEQ ID NO: 24 - DNA, 84 bp, synthetic construct
  source 1..84: mol_type = other DNA
tatgaggccggcatgacactcggcggcaagatcctgttcttcctgttcctgctgctccctctgagccccttcagcctgatcttc

SEQ ID NO: 25 - protein, 24 aa, synthetic construct
TEISCCTLSSEENEYLPRPEWQLQ

SEQ ID NO: 26 - DNA, 72 bp, synthetic construct
  source 1..72: mol_type = other DNA
accgagatcagctgctgcaccctgagcagcgaggaaaacgagtacctgcctagacctgagtggcagctgcag

SEQ ID NO: 27 - protein, 61 aa, synthetic construct
VPFRELKNVSVLEGLRQGRLGGPCSCHCPRPSQARLTPVDVAGPFLCLGDPGLFPPVKSSI

SEQ ID NO: 28 - DNA, 183 bp, synthetic construct
  source 1..183: mol_type = other DNA
gtccccttcagagagctgaagaacgtttccgtgctggaaggcctgagacagggcagacttggcggcccttgtagctgtcactgccccagacctagtcaggccagactgacacctgtggatgtggccggacctttcctgtgtctgggagatcctggcctgtttccacctgtgaagtccagcatc

SEQ ID NO: 29 - protein, 33 aa, synthetic construct
TGGKSTCSAPGPQSLPSTPFSTYPQWVILITEL

SEQ ID NO: 30 - DNA, 99 bp, synthetic construct
  source 1..99: mol_type = other DNA
acaggcggcaagtccacatgttctgcccctggacctcagagcctgcctagcacacccttcagcacataccctcagtgggtcatcctgatcaccgaactc

SEQ ID NO: 31 - protein, 59 aa, synthetic construct
GMECTLGQVGAPSPRREEDGWRGGHSRFKADVPAPQGPCWGGQPGSAPSSAPPEQSLLD

SEQ ID NO: 32 - DNA, 177 bp, synthetic construct
  source 1..177: mol_type = other DNA
ggcatggaatgcaccctgggacaagtgggagccccatctcctagaagagaagaggatggctggcgcggaggccactctagattcaaagctgatgtgcccgctcctcagggcccttgttggggaggacaacctggatctgccccatcttctgccccacctgaacagtccctgctggat

SEQ ID NO: 33 - protein, 27 aa, synthetic construct
WKFEMSYTVGGPPPHVHARPRHWKTDR

SEQ ID NO: 34 - DNA, 81 bp, synthetic construct
  source 1..81: mol_type = other DNA
tggaagttcgagatgagctacaccgtcggcggacctccacctcatgttcatgccagacctcggcactggaaaaccgacaga

SEQ ID NO: 35 - protein, 49 aa, synthetic construct
DGHSYTSKVNCLLLQDGFHGCVSITGAAGRRNLSIFLFLMLCKLEFHAC

SEQ ID NO: 36 - DNA, 147 bp, synthetic construct
  source 1..147: mol_type = other DNA
gatggccacagctacaccagcaaagtgaactgcctcctgctgcaggatggcttccacggctgtgtgtctattactggcgccgctggcagacggaacctgagcatctttctgtttctgatgctgtgcaagctcgagttccacgcctgc

SEQ ID NO: 37 - protein, 9 aa, synthetic construct
KIQNKNCPD

SEQ ID NO: 38 - DNA, 27 bp, synthetic construct
  source 1..27: mol_type = other DNA
aagatccagaacaagaactgccccgac

SEQ ID NO: 39 - protein, 50 aa, synthetic construct
FKKFDGPCGERGGGRTARALWARGDSVLTPALDPQTPVRAPSLTRAAAAV

SEQ ID NO: 40 - DNA, 150 bp, synthetic construct
  source 1..150: mol_type = other DNA
ttcaagaagttcgacggcccttgcggagaaagaggcggaggcagaacagctagagccctttgggctagaggcgacagcgttctgacaccagctctggaccctcagacacctgttagggcccctagcctgacaagagctgccgccgctgtg

SEQ ID NO: 41 - protein, 75 aa, synthetic construct
HYKLIQQPISLFSITDRLHKTFSQLPSVHLCSITFQWGHPPIFCSTNDICVTANFCISVTFLKPCFLLHEASASQ

SEQ ID NO: 42 - DNA, 225 bp, synthetic construct
  source 1..225: mol_type = other DNA
cactacaagctgatccagcagccaatcagcctgttcagcatcaccgaccggctgcacaagacattcagccagctgccaagcgtgcacctgtgctccatcaccttccagtggggacaccctcctatcttttgctccaccaacgacatctgcgtgaccgccaacttctgtatcagcgtgaccttcctgaagccttgctttctgctgcacgaggccagcgcctctcag

SEQ ID NO: 43 - protein, 95 aa, synthetic construct
CHLFLQPQVGTPPPHTASARAPSGPPHPHESCPAGRRPARAAQTCARRQHGLPGCEEAGTARVPSLHLHLHQAALGAGRGRGWGEACAQVPPSRG

SEQ ID NO: 44 - DNA, 285 bp, synthetic construct
  source 1..285: mol_type = other DNA
tgccacttgtttctgcagccccaagtgggcacacctcctccacatacagcctctgctagagcacctagcggccctccacatcctcacgaatcttgtcctgccggaagaaggcctgccagagccgctcaaacatgtgccagacgacagcacggactgcctggatgtgaagaggctggaacagccagagtgcctagcctgcacctccatctgcatcaggctgctcttggagccggaagaggtagaggatggggcgaagcttgtgctcaggtgccaccttctagaggc

SEQ ID NO: 45 - protein, 14 aa, synthetic construct
VLRFLDLKVRYLHS

SEQ ID NO: 46 - DNA, 42 bp, synthetic construct
  source 1..42: mol_type = other DNA
gtgctgagattcctggacctgaaagtgcgctacctgcacagc

SEQ ID NO: 47 - protein, 22 aa, synthetic construct
QWQHYHRSGEAAGTPLWRPTRN

SEQ ID NO: 48 - DNA, 66 bp, synthetic construct
  source 1..66: mol_type = other DNA
cagtggcagcactatcacagatctggcgaagccgccggaacacccctttggaggccaacaagaaac

SEQ ID NO: 49 - protein, 90 aa, synthetic construct
VPFRELKNQRTAQGAPGIHHAASPVAANLCDPARHAQHTRIPCGAGQVRAGRGPEAGGGVLQPQRPAPEKPGCPCRRGQPRLHTVKMWRA

SEQ ID NO: 50 - DNA, 270 bp, synthetic construct
  source 1..270: mol_type = other DNA
gtgcccttccgggaactgaagaaccagagaacagctcagggcgctcctggaatccaccatgctgcttctccagtggccgccaacctgtgtgatcctgccagacatgcccagcacaccaggattccttgtggcgctggacaagtgcgcgctggaagaggacctgaagcaggcggaggtgttctgcaacctcaaagacccgctcctgagaagcctggctgcccttgcagaagaggacagcctagactgcacaccgtgaaaatgtggcgagcc

SEQ ID NO: 51 - protein, 16 aa, synthetic construct
VAMMVPDRQVHYDFGL

SEQ ID NO: 52 - DNA, 48 bp, synthetic construct
  source 1..48: mol_type = other DNA
gtggccatgatggtgcccgatagacaggtccactacgactttggactg

SEQ ID NO: 53 - protein, 17 aa, synthetic construct
GVPGDSTRRAVRRMNTF

SEQ ID NO: 54 - DNA, 51 bp, synthetic construct
  source 1..51: mol_type = other DNA
ggcgtgccaggcgatagcactcggagagccgtcagacggatgaacaccttt

SEQ ID NO: 55 - protein, 24 aa, synthetic construct
YEAGMTLGEKFRVGNCKHLKMTRP

SEQ ID NO: 56 - DNA, 72 bp, synthetic construct
  source 1..72: mol_type = other DNA
tacgaagccgggatgaccctgggcgagaagttcagagtgggcaactgcaagcacctgaagatgacccggcct

SEQ ID NO: 57 - protein, 16 aa, synthetic construct
NSKMALNSEALSVVSE

SEQ ID NO: 58 - RNA, 48 nt, synthetic construct
  source 1..48: mol_type = other RNA
aacagcaagatggccctgaatagcgaggccctgtctgtggtgtctgaa

SEQ ID NO: 59 - protein, 16 aa, synthetic construct
CGASACDVSLIAMDSA

SEQ ID NO: 60 - DNA, 48 bp, synthetic construct
  source 1..48: mol_type = other DNA
tgtggcgcctctgcctgtgacgtgtccctgatcgctatggactccgcc

SEQ ID NO: 61 - protein, 17 aa, synthetic construct
FVQGKDWGVKKFIRRDF

SEQ ID NO: 62 - DNA, 51 bp, synthetic construct
  source 1..51: mol_type = other DNA
tttgtgcagggcaaagactggggcgtgaagaagttcatccggcgggacttc

SEQ ID NO: 63 - protein, 67 aa, synthetic construct
YAYKDFLWCFPFSLVFLQEIQICCHVSCLCCICCSTRICLGCLLELFLSRALRALHVLWNGFQLHCQ

SEQ ID NO: 64 - DNA, 201 bp, synthetic construct
  source 1..201: mol_type = other DNA
tacgcctacaaggacttcctgtggtgcttccccttctctctggtgttcctgcaagagatccagatctgctgtcatgtgtcctgcctgtgctgcatctgctgtagcaccagaatctgcctgggctgtctgctggaactgttcctgagcagagccctgagagcactgcacgtgctgtggaacggattccagctgcactgccag

SEQ ID NO: 65 - protein, 16 aa, synthetic construct
TEYNQKLQVNQFSESK

SEQ ID NO: 66 - DNA, 48 bp, synthetic construct
  source 1..48: mol_type = other DNA
accgagtacaaccagaaactgcaagtgaaccagttcagcgagagcaag

SEQ ID NO: 67 - protein, 16 aa, synthetic construct
SLYHREKQLIAMDSAI

SEQ ID NO: 68 - DNA, 48 bp, synthetic construct
  source 1..48: mol_type = other DNA
agcctgtaccaccgggaaaagcagctcattgccatggacagcgccatc

SEQ ID NO: 69 - protein, 14 aa, synthetic construct
CEERGAAGSLISCE

SEQ ID NO: 70 - DNA, 42 bp, synthetic construct
  source 1..42: mol_type = other DNA
tgcgaagagagaggcgccgcaggatctctgatctcctgcgaa

SEQ ID NO: 71 - protein, 16 aa, synthetic construct
TMPAILKLQKNCLLSL

SEQ ID NO: 72 - DNA, 48 bp, synthetic construct
  source 1..48: mol_type = other DNA
acaatgcccgccatcctgaagctgcagaagaattgcctcctaagcctg

SEQ ID NO: 73 - protein, 144 aa, synthetic construct
RTALTHNQDFSIYRLCCKRGSLCHASQARSPAFPKPVRPLPAPITRITPQLGGQSDSSQPLLTTGRPQGWQDQALRHTQQASPASCATITIPIHSAALGDHSGDPGPAWDTCPPLPLTTLIPRAPPPYGDSTARSWPSRCGPLG

SEQ ID NO: 74 - DNA, 432 bp, synthetic construct
  source 1..432: mol_type = other DNA
cgaaccgctctgacacacaaccaggacttcagcatctacagactgtgttgcaagcggggctccctgtgccatgcaagccaagctagaagccccgcctttcctaaacctgtgcgacctctgccagctccaatcaccagaattacccctcagctcggcggccagagcgattcatctcaacctctgctgaccaccggcagacctcaaggctggcaagaccaagctctgagacacacccagcaggctagccctgcctcttgtgccaccatcacaatccccatccactctgccgctctgggcgatcattctggcgatcctggaccagcctgggacacatgtcctccactgccactcacaacactgatccctagggctcctccaccttacggcgattctaccgctagaagctggcccagcagatgtggaccactcgga

SEQ ID NO: 75 - protein, 32 aa, synthetic construct
GNTTLQQLGEASQAPSGSLIPLRLPLLWEVRG

SEQ ID NO: 76 - DNA, 96 bp, synthetic construct
  source 1..96: mol_type = other DNA
ggcaacacaaccctccagcaactgggagaagcctctcaggctcctagcggctctctgatccctctcagactgcctctcctgtgggaagttcggggc

SEQ ID NO: 77 - protein, 17 aa, synthetic construct
QPDSFAALHSSLNELGE

SEQ ID NO: 78 - DNA, 51 bp, synthetic construct
  source 1..51: mol_type = other DNA
cagcctgattcttttgccgcactgcacagctccctgaacgagctgggagag

SEQ ID NO: 79 - protein, 17 aa, synthetic construct
IARELHQFAFDLLIKSH

SEQ ID NO: 80 - DNA, 51 bp, synthetic construct
  source 1..51: mol_type = other DNA
atcgctagagagctgcaccagttcgccttcgacctgctgatcaagagccac

SEQ ID NO: 81 - protein, 17 aa, synthetic construct
FVQGKDWGLKKFIRRDF

SEQ ID NO: 82 - DNA, 51 bp, synthetic construct
  source 1..51: mol_type = other DNA
ttcgtgcaaggcaaggattggggcctcaaaaagtttatccgcagagacttc

SEQ ID NO: 83 - protein, 70 aa, synthetic construct
WGMELAASRRFSWDHHSAGGPPRVPSVRSGAAQVQPKDPLPLRTLAGCLARTAHLRPGAESLPQPQLHCT

SEQ ID NO: 84 - DNA, 210 bp, synthetic construct
  source 1..210: mol_type = other DNA
tggggcatggaactggccgccagcagaagattcagctgggatcatcatagcgcaggcggcccacctagagtgccatctgttagaagcggagctgcccaggtgcagcctaaagatcctctgccactgagaacactggccggctgccttgctagaacagcccatcttagacctggcgccgagtctctgcctcagccacaactgcactgtacc

SEQ ID NO: 85 - protein, 69 aa, synthetic construct
LWFQSSELSPTGAPWPSRRPTWRGTTVSPRTATSSARTCCGTKWPSSQEAALGLGSGLLRFSCGTAAIR

SEQ ID NO: 86 - DNA, 207 bp, synthetic construct
  source 1..207: mol_type = other DNA
ctgtggttccagtccagcgagctgtctcctactggtgccccttggccatctagacgccctacttggagaggcaccaccgtgtcaccaagaaccgccacaagcagcgccagaacctgttgtggcacaaagtggccctccagccaagaagccgctctcggacttggaagcggactgctgaggttctcttgtggaaccgccgccattcgg

SEQ ID NO: 87 - protein, 16 aa, synthetic construct
KMHFSLKEHPPPPCPP

SEQ ID NO: 88 - DNA, 48 bp, synthetic construct
  source 1..48: mol_type = other DNA
aagatgcactttagcctgaaagaacaccctccaccaccttgtcctcca

SEQ ID NO: 89 - protein, 43 aa, synthetic construct
EAFQRAAGEGGPGRGGARRGARVLQSPFCRAGAGEWLGHQSLR

SEQ ID NO: 90 - DNA, 129 bp, synthetic construct
  source 1..129: mol_type = other DNA
gaggctttccaaagagctgctggcgaaggcggacctggtagaggtggtgctagaagaggtgctagggtgctgcagagcccattctgtagagcaggcgcaggcgaatggctgggccatcagagtctgaga

SEQ ID NO: 91 - protein, 20 aa, synthetic construct
HVVGYGHLDTSGSSSSSSWP

SEQ ID NO: 92 - DNA, 60 bp, synthetic construct
  source 1..60: mol_type = other DNA
catgtcgtcggctacggccacctggatacaagcggaagcagctctagctccagctggcct

SEQ ID NO: 93 - protein, 35 aa, synthetic construct
NSKMALNSLNSIDDAQLTRIAPPRSHCCFWEVNAP

SEQ ID NO: 94 - DNA, 105 bp, synthetic construct
  source 1..105: mol_type = other DNA
aactcaaaaatggctctgaacagcctgaactccatcgacgacgcccagctgacaagaatcgcccctcctagatctcactgctgcttttgggaagtgaacgcccca